(19) United States
(12) Patent Application Publication
(10) Pub. No.: US 2016/0237044 A1
(43) Pub. Date: Aug. 18, 2016

(54) SUBSTITUTED PHENYLALANINE DERIVATIVES

(71) Applicant: BAYER PHARMA AKTIENGESELLSCHAFT, Berlin (DE)

(72) Inventors: Ulrike RÖHN, Berlin (DE); Manuel ELLERMANN, Berlin (DE); Julia STRASSBURGER, Wuppertal (DE); Astrid WENDT, Syke (DE); Susanne RÖHRIG, Hilden (DE); Robert Alan WEBSTER, Wuppertal (DE); Martina Victoria SCHMIDT, Köln (DE); Adrian TERSTEEGEN, Wuppertal (DE); Kristin BEYER, Kensington, CA (US); Martina SCHÄFER, Berlin (DE); Anja BUCHMÜLLER, Essen (DE); Christoph GERDES, Köln (DE); Michael SPERZEL, Kierspe (DE); Steffen SANDMANN, Essen-Heisingen (DE); Stefan HEITMEIER, Wülfrath (DE); Alexander HILLISCH, Solingen (DE); Jens ACKERSTAFF, Düsseldorf (DE); Carsten TERJUNG, Bochum (DE)

(73) Assignee: Bayer Pharma Aktiengesellschaft, Berlin (DE)

(21) Appl. No.: 15/024,355

(22) PCT Filed: Sep. 24, 2014
(86) PCT No.: PCT/EP2014/070316

§ 371 Date: Mar. 23, 2016

(30) Foreign Application Priority Data
Sep. 26, 2013 (EP) 13186057.9

(51) Int. Cl. C07D 257/04; C07D 413/12; C07D 237/32; C07D 403/12; C07D 235/26; C07D 249/08; C07D 401/12; C07D 271/07

(52) U.S. Cl. CPC C07D 257/04; C07D 401/12; C07D 413/12; C07D 271/07; C07D 403/12; C07D 235/26; C07D 249/08; C07D 237/32; C07B 2200/07

(57) ABSTRACT

The invention relates to substituted phenylalanine derivatives and to processes for preparation thereof, and to the use thereof for production of medicaments for treatment and/or prophylaxis of diseases, especially of cardiovascular disorders and/or severe perioperative blood loss. TRG416065

The invention relates to substituted phenylalanine derivatives and to processes for preparation thereof, and to the use thereof for production of medicaments for treatment and/or prophylaxis of diseases, especially of cardiovascular disorders and/or severe perioperative blood loss.

Blood coagulation is a protective mechanism of the organism which helps to “seal” defects in the wall of the blood vessels quickly and reliably. Thus, loss of blood can be avoided or kept to a minimum. Haemostasis after injury of the blood vessels is effected mainly by the coagulation system in which an enzymatic cascade of complex reactions of plasma proteins is triggered. Numerous blood coagulation factors are involved in this process, each of which factors converts, on activation, the respectively next inactive precursor into its active form. At the end of the cascade comes the conversion of soluble fibrinogen into insoluble fibrin, resulting in the formation of a blood clot. In blood coagulation, traditionally the intrinsic and the extrinsic system, which end in a final joint reaction path, are distinguished. Here, factors Xa and IIa (thrombin) play key roles: Factor Xa bundles the signals of the two coagulation paths since it is formed both via factor VIIa/tissue factor (extrinsic path) and via the tenase complex (intrinsic path) by conversion of factor X. The activated serine protease Xa cleaves prothrombin to thrombin which, via a series of reactions, transduces the impulses from the cascade to the coagulation state of the blood.

In the more recent past, the traditional theory of two separate regions of the coagulation cascade (extrinsic and intrinsic path) has been modified owing to new findings: In these models, coagulation is initiated by binding of activated factor VIIa to tissue factor (TF). The resulting complex activates factor X, which in turn leads to generation of thrombin with subsequent production of fibrin and platelet activation (via PAR-1) as injury-sealing end products of haemostasis. Compared to the subsequent amplification/propagation phase, the thrombin production rate is low and as a result of the occurrence of TFPI as inhibitor of the TF-FVIIa-FX complex is limited in time.

A central component of the transition from initiation to amplification and propagation of coagulation is factor XIa. In positive feedback loops, thrombin activates, in addition to factor V and factor VIII, also factor XI to factor XIa, whereby factor IX is converted into factor IXa, thus, via the factor IXa/factor VIIIa complex generated in this manner, rapidly producing relatively large amounts of factor Xa. This triggers the production of large amounts of thrombin, leading to strong thrombus growth and stabilizing the thrombus.

The formation of a thrombus or blood clot is counter-regulated by fibrinolysis. Activation of plasminogen by tissue plasminogen activator (tPA) results in formation of the active serine protease, plasmin, which cleaves polymerized fibrin and thus forms the thrombus. This process is referred to as fibrinolysis—with plasmin as key enzyme.

Uncontrolled activation of the coagulation system or defects in the inhibition of the activation processes may cause formation of local thromboses or embolisms in vessels (arteries, veins, lymph vessels) or heart chambers. This may lead to serious thrombotic or thromboembolic disorders. In addition, systemic hypercoagulability may lead to consumption coagulopathy in the context of a disseminated intravasal coagulation.

In the course of many cardiovascular and metabolic disorders, there is an increased tendency for coagulation and platelet activation owing to systemic factors such as hyperlipidaemia, diabetes or smoking, owing to changes in blood flow with stasis, for example in atrial fibrillation, or owing to pathological changes in vessel walls, for example endothelial dysfunctions or atherosclerosis. This unwanted and excessive haemostasis may, by formation of fibrin- and platelet-rich thrombi, lead to thromboembolic disorders and thrombotic complications with life-threatening conditions.

Thromboembolic disorders are the most frequent cause of morbidity and mortality in most industrialized countries [Heart Disease: A Textbook of Cardiovascular Medicine, Eugene Braunwald, 5th edition, 1997, W.B. Saunders Company, Philadelphia].

The anticoagulants known from the prior art, for example substances for inhibiting or preventing blood coagulation, have various, frequently grave disadvantages. Accordingly, in practice, efficient treatment methods or the prophylaxis of thrombotic/thromboembolic disorders are found to be very difficult and unsatisfactory.

In the therapy and prophylaxis of thromboembolic disorders, use is made, firstly, of heparin which is administered parenterally or subcutaneously. Because of more favourable pharmacokinetic properties, preference is these days increasingly given to low-molecular-weight heparin; however, the known disadvantages described hereinbelow encountered in heparin therapy cannot be avoided either in this manner. Thus, heparin is orally ineffective and has only a comparatively short half-life. In addition, there is a high risk of bleeding, there may in particular be cerebral haemorrhages and bleeding in the gastrointestinal tract, and there may be thrombopaenia, alopecia medicomentosa or osteoporosis [Pschyrembel, Klinisches Wörterbuch [clinical dictionary], 257th edition, 1994, Walter de Gruyter Verlag, page 610, keyword “Heparin”; Römpp Lexikon Chemie, Version 1.5, 1998, Georg Thieme Verlag Stuttgart, keyword “Heparin”]. Low-molecular-weight heparins do have a lower probability of leading to the development of heparin-induced thrombocytopaenia; however, they can also only be administered subcutaneously. This also applies to fondaparinux, a synthetically produced selective factor Xa inhibitor having a long half-life.

A second class of anticoagulants are the vitamin K antagonists. These include, for example, 1,3-indanediones and in particular compounds such as warfarin, phenprocoumon, dicumarol and other coumarin derivatives which non-selectively inhibit the synthesis of various products of certain vitamin K-dependent coagulation factors in the liver. Owing to the mechanism of action, the onset of action is only very slow (latency to the onset of action 36 to 48 hours). The compounds can be administered orally; however, owing to the high risk of bleeding and the narrow therapeutic index complicated individual adjustment and monitoring of the patient are required [J. Hirsh, J. Dalen, D. R. Anderson et al., “Oral anticoagulants: Mechanism of action, clinical effectiveness, and optimal therapeutic range” Chest 2001, 119, 8S-21S; J. Ansell, J. Hirsh, J. Dalen et al., “Managing oral anticoagulant therapy” Chest 2001, 119, 22S-38S; P. S. Crowther et al., “Interactions of warfarin with drugs and food” Ann. Intern. Med. 1994, 121, 676-683]. In addition, other side-effects such as gastrointestinal problems, hair loss and skin necroses have been described.

More recent approaches for oral anticoagulants are in various phases of clinical evaluation or in clinical use, but they have also shown disadvantages, for example highly variable bioavailability, liver damage and bleeding complications.

For antithrombotic medicaments, the therapeutic width is of central importance: The interval between the therapeutically active dose for coagulation inhibition and the dose where bleeding may occur should be as large as possible so that maximum therapeutic activity is achieved at a minimum risk profile.

In various in vivo models with, for example, antibodies as factor XIa inhibitors, but also in factor

XIa knock-out models, the antithrombotic effect with small/no prolongation of bleeding time or extension of blood volume was confirmed. In clinical studies, elevated factor XIa concentrations were associated with an increased event rate. However, factor XI deficiency (haemophilia C), in contrast to factor VIIIa or factor IXa (haemophilia A and B, respectively), did not lead to spontaneous bleeding and was only noticed during surgical interventions and traumata. Instead, protection against certain thromboembolic events was found.

In the event of hyperfibrinolytic states, there is inadequate wound closure, which causes severe, sometimes life-threatening, bleeding. This bleeding can be stopped by the inhibition of fibrinolysis with antifibrinolytics, by which plasmin activity is reduced. Corresponding effects with the plasminogen inhibitor tranexamic acid have been shown in various clinical studies.

It is therefore an object of the present invention to provide novel compounds for treatment and/or prophylaxis of cardiovascular disorders and/or severe perioperative blood loss in man and animals, said compounds having a wide therapeutic range. WO89/11852 describes, inter alia, substituted phenylalanine derivatives for treatment of pancreatitis, and WO 2007/070816 describes substituted thiophene derivatives as factor XIa inhibitors.

The invention provides compounds of the formula

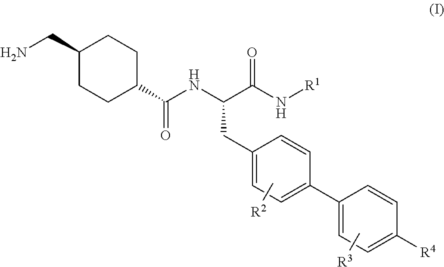

in which
R¹ represents a group of the formula

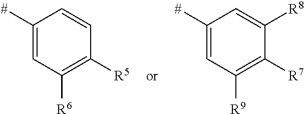

where # is the point of attachment to the nitrogen atom,
R⁵ represents 5-membered heteroaryl,
   where heteroaryl may be substituted by a substituent selected from the group consisting of oxo, chlorine, cyano, hydroxy and C₁-C₃-alkyl,
      in which alkyl may be substituted by 1 to 3 substituents independently of one another selected from the group consisting of hydroxy, amino, hydroxycarbonyl and methoxy,
      or
      in which alkyl may be substituted by 1 to 7 fluorine substituents,
      or
      in which alkyl is substituted by a substituent selected from the group consisting of hydroxy, amino, hydroxycarbonyl and methoxy, and in which alkyl is additionally substituted by 1 to 6 fluorine substituents,
R⁶ represents hydrogen, fluorine or chlorine,
R⁷ and R⁸ together with the carbon atoms to which they are attached form a 5-membered heterocycle,
   where the heterocycle may be substituted by 1 to 2 substituents independently of one another selected from the group consisting of oxo, chlorine, cyano, hydroxy, C₁-C₃-alkyl, pyrazolyl and pyridyl,
      in which alkyl may be substituted by 1 to 3 substituents independently of one another selected from the group consisting of hydroxy, amino, hydroxycarbonyl and methoxy,
      or
      in which alkyl may be substituted by 1 to 7 fluorine substituents,
      or
      in which alkyl is substituted by a substituent selected from the group consisting of hydroxy, amino, hydroxycarbonyl and methoxy, and in which alkyl is additionally substituted by 1 to 6 fluorine substituents,
R⁹ represents hydrogen, fluorine or chlorine,
R² represents hydrogen, fluorine, chlorine, methyl or methoxy,
R³ represents hydrogen, fluorine, chlorine, C₁-C₄-alkyl, methoxy or trifluoromethyl,
R⁴ represents amino, cyano, C₁-C₃-alkoxy, C₁-C₃-alkylamino, C₁-C₃-alkoxycarbonyl, C₁-C₃-alkylcarbonylamino, C₁-C₃-alkylsulphonyl, —S(O)₂NR¹⁰R¹¹ or 5- to 7-membered heterocyclyl which is attached via a nitrogen atom,
   where alkoxy may be substituted by 1 to 2 substituents independently of one another selected from the group consisting of fluorine, hydroxy, amino, hydroxycarbonyl, C₁-C₃-alkoxy, C₁-C₃-alkylamino, difluoromethyl, trifluoromethyl, —(OCH₂CH₂)ₙ—OCH₃, —(OCH₂CH₂)ₘ—OH, morpholinyl, piperidinyl and pyrrolidinyl,
      in which n is a number from 1 to 6,
      in which m is a number from 1 to 6
   and
   where alkylcarbonylamino may be substituted by a —NH(CO)CH₂NH(CO)CH₂NH₂ substituent,
   and
   where heterocyclyl may be substituted by 1 to 2 substituents independently of one another selected from the group consisting of oxo, chlorine, cyano, hydroxy and C₁-C₄-alkyl,
   and
   where R¹⁰ represents hydrogen, C₁-C₃-alkyl, C₃-C₆-cycloalkyl, benzyl or 4- to 8-membered heterocyclyl which is attached via a carbon atom,
      in which alkyl may be substituted by 1 to 2 substituents independently of one another selected from the group consisting of hydroxy, amino, hydroxycarbonyl and methoxy,
      and
      in which cycloalkyl may be substituted by 1 to 2 substituents independently of one another selected from the group consisting of oxo, fluorine, hydroxy, amino, C₁-C₄-alkyl and C₁-C₃-alkylamino,
      and
      in which heterocyclyl may be substituted by 1 to 2 substituents independently of one another selected from the group consisting of oxo, chlorine, cyano, hydroxy and C₁-C₄-alkyl,
   R¹¹ represents hydrogen or C₁-C₃-alkyl,
   or
   R¹⁰ and R¹¹ together with the nitrogen atom to which they are attached form a 4- to 7-membered heterocycle,
      in which the heterocycle may be substituted by 1 to 2 substituents selected independently from the group consisting of oxo, fluorine, hydroxy, amino, hydroxycarbonyl, C₁-C₄-alkyl, C₁-C₃-alkylamino, difluoromethyl, trifluoromethyl, 2,2,2-trifluoroeth-1-yl, C₁-C₄-alkoxy carbonyl, aminocarbonyl and C₁-C₃-alkylaminocarbonyl,

and the salts thereof, the solvates thereof and the solvates of the salts thereof.

Compounds according to the invention are the compounds of the formula (I) and the salts, solvates and solvates of the salts thereof, and also the compounds encompassed by formula (I) and specified hereinafter as working example(s), and the salts, solvates and solvates of the salts thereof, to the extent that the compounds encompassed by formula (I) and specified hereinafter are not already salts, solvates and solvates of the salts.

The compounds of the invention may, depending on their structure, exist in different stereoisomeric forms, i.e. in the form of configurational isomers or else, if appropriate, of conformational isomers (enantiomers and/or diastereomers, including those in the case of atropisomers). The present invention therefore encompasses the enantiomers and diastereomers, and the respective mixtures thereof. The stereoisomerically uniform constituents can be isolated from such mixtures of enantiomers and/or diastereomers in a known manner; chromatography processes are preferably used for this, especially HPLC chromatography on an achiral or chiral phase.

If the compounds of the invention can occur in tautomeric forms, the present invention encompasses all the tautomeric forms.

The present invention also encompasses all suitable isotopic variants of the compounds of the invention. An isotopic variant of a compound of the invention is understood here to mean a compound in which at least one atom within the compound of the invention has been exchanged for another atom of the same atomic number, but with a different atomic mass from the atomic mass which usually or predominantly occurs in nature. Examples of isotopes which can be incorporated into a compound of the invention are those of hydrogen, carbon, nitrogen, oxygen, phosphorus, sulphur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of a compound of the invention, especially those in which one or more radioactive isotopes have been incorporated, may be beneficial, for example, for the examination of the mechanism of action or of the active ingredient distribution in the body; due to comparatively easy preparability and detectability, especially compounds labelled with ³H or ¹⁴C isotopes are suitable for this purpose. In addition, the incorporation of isotopes, for example of deuterium, may lead to particular therapeutic benefits as a consequence of greater metabolic stability of the compound, for example an extension of the half-life in the body or a reduction in the active dose required; such modifications of the compounds of the invention may therefore in some cases also constitute a preferred embodiment of the present invention. Isotopic variants of the compounds of the invention can be prepared by the processes known to those skilled in the art, for example by the methods described further down and the procedures described in the working examples, by using corresponding isotopic modifications of the respective reagents and/or starting compounds.

Preferred salts in the context of the present invention are physiologically acceptable salts of the compounds according to the invention. However, the invention also encompasses salts which themselves are unsuitable for pharmaceutical applications but which can be used, for example, for the isolation or purification of the compounds according to the invention.

Physiologically acceptable salts of the compounds according to the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine, N-methylpiperidine and choline.

Solvates in the context of the invention are described as those forms of the compounds according to the invention which form a complex in the solid or liquid state by coordination with solvent molecules. Hydrates are a specific form of the solvates in which the coordination is with water.

The present invention additionally also encompasses prodrugs of the compounds of the invention. The term “prodrugs” encompasses compounds which for their part may be biologically active or inactive but are converted during their residence time in the body into compounds according to the invention (for example by metabolism or hydrolysis).

The two ways (A) and (B) of representing a 1,4-disubstituted cyclohexyl derivative shown below are equivalent to one another and identical, and in both cases describe a trans-1,4-disubstituted cyclohexyl derivative.

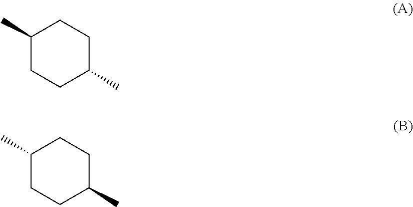

This applies especially to the structural element of tranexamamide, for example N-[(trans-4-{[(tert-butoxycarbonyl)amino]methyl}cyclohexyl)carbonyl and trans-4-(aminomethyl)-cyclohexyl]carbonyl}. In the present invention, representation (A) is used.

The three ways (C), (D) and (E) of representing tautomers of a triazole derivative shown below are equivalent to one another and identical and in all cases describe a 1,4-disubstituted triazole derivative.

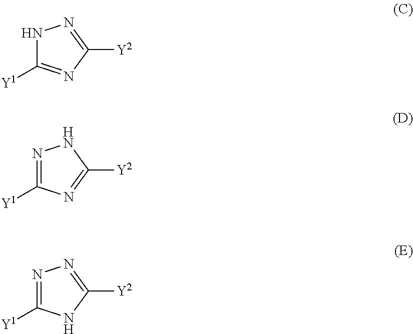

This applies especially to the following structural elements: 1H-1,2,4-triazol-3-yl, 1H-1,2,4-triazol-5-yl, 4H-1,2,4-triazol-3-yl and 4H-1,2,4-triazol-5-yl. Y¹ and Y² here are different substituents.

The two ways (F) and (G) of representing tautomers of a tetrazole derivative shown below are equivalent to one another and identical and in all cases describe a tetrazole derivative.

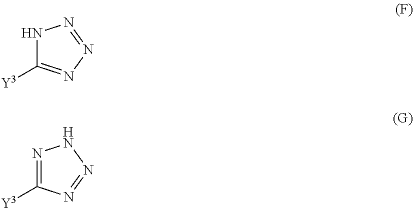

This applies especially to the following structural elements: 1H-tetrazol-5-yl and 2H-tetrazol-5-yl. Y³ here is the remainder of the compound.

The compounds according to the invention of the formula

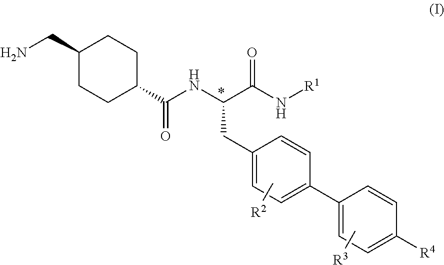

and all L-phenylalanine intermediates are described as the (S) configuration at the stereocentre marked with an * in the above formulae, since L-phenylalanine derivatives are introduced into the synthesis as central units. In the preparation of the compounds according to the invention, the coupling of the L-phenylalanine intermediates with the amine H₂N—R¹ can result in partial epimerization at the stereocentre marked with an *. Thus, a mixture of the compounds according to the invention of (S) enantiomer and (R) enantiomer can arise. The main component is the (S) enantiomer depicted in each case. The mixtures of (S) enantiomer and (R) enantiomer can be separated into their enantiomers by methods known to those skilled in the art, for example by chromatography on a chiral phase.

The enantiomers can be separated either directly after the coupling of the L-phenylalanine intermediates with the amine H₂N—R¹ or at a later synthesis intermediate stage, or else the inventive compounds can be separated themselves. Preference is given to the separation of the enantiomers directly after the coupling of the L-phenylalanine intermediates with the amine H₂N—R¹.

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is used here synonymously with the term “treatment”.

The terms “prevention”, “prophylaxis” and “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

In the context of the present invention, unless specified otherwise, the substituents are defined as follows:

Alkyl represents a straight-chain or branched alkyl radical having 1 to 4 carbon atoms, preferably 1 to 3 carbon atoms, by way of example and with preference methyl, ethyl, n-propyl, isopropyl, 2-methylprop-1-yl, n-butyl and tert-butyl.

Alkoxy represents a straight-chain or branched alkoxy radical having 1 to 4 carbon atoms, preferably 1 to 3 carbon atoms, by way of example and with preference methoxy, ethoxy, n-propoxy, isopropoxy, 2-methylprop-1-oxy, n-butoxy and tert-butoxy.

Alkylamino represents an amino group having one or two independently selected, identical or different, straight-chain or branched alkyl radicals each having 1 to 3 carbon atoms, for example and with preference methylamino, ethylamino, n-propylamino, isopropylamino, N,N-dimethylamino, N,N-diethylamino, N-ethyl-N-methylamino, N-methyl-N-n-propylamino, N-isopropyl-N-n-propylamino and N,N-diisopropylamino. C₁-C₃-Alkylamino represents, for example, a monoalkylamino radical having 1 to 3 carbon atoms or a dialkylamino radical having 1 to 3 carbon atoms in each alkyl radical.

Alkoxycarbonyl represents a straight-chain or branched alkoxy radical attached via a carbonyl group and having 1 to 4 carbon atoms, preferably 1 to 3 carbon atoms, for example and with preference methoxycarbonyl, ethoxycarbonyl, n-propoxycarbonyl, isopropoxycarbonyl, n-butoxycarbonyl and tert-butoxycarbonyl.

Alkylcarbonylamino represents a straight-chain or branched alkyl radical attached via a carbonylamino group and having 1 to 3 carbon atoms, for example and with preference methylcarbonylamino, ethylcarbonylamino, n-propylcarbonylamino and isopropylcarbonylamino

Alkylaminocarbonyl represents an amino group having one or two independently selected, identical or different, straight-chain or branched alkyl substituents each having 1 to 3 carbon atoms, attached via a carbonyl group, for example and with preference methylaminocarbonyl, ethylaminocarbonyl, n-propylaminocarbonyl, isopropylaminocarbonyl, N,N-dimethylaminocarbonyl, N,N-diethylaminocarbonyl, N-ethyl-N-methylaminocarbonyl, N-methyl-N-n-propylaminocarbonyl, N-isopropyl-N-n-propylaminocarbonyl and N,N-diisopropylaminocarbonyl. C₁-C₃-Alkylaminocarbonyl represents, for example, a monoalkylaminocarbonyl radical having 1 to 3 carbon atoms or a dialkylaminocarbonyl radical having 1 to 3 carbon atoms in each alkyl substituent.

Alkylsulphonyl represents a straight-chain or branched alkyl radical attached via a sulphonyl group and having 1 to 3 carbon atoms, for example and with preference methylsulphonyl, ethylsulphonyl, n-propylsulphonyl and isopropylsulphonyl.

Cycloalkyl represents a monocyclic cycloalkyl group having 3 to 6 carbon atoms, preferred examples of cycloalkyl being cyclopropyl, cyclobutyl, cyclopentyl and cyclohexyl.

5- to 7-membered heterocyclyl attached via a nitrogen atom in the definition of the R⁴ radical represents a saturated or partly unsaturated monocyclic radical attached via a nitrogen atom and having 5 to 7 ring atoms, preferably 5 or 6 ring atoms, and up to 3 heteroatoms and/or hetero groups, preferably 1 or 2 heteroatoms and/or hetero groups, from the group consisting of S, O, N, SO and SO₂, where a nitrogen atom may also form an N-oxide, for example and with preference pyrrolidinyl, morpholinyl, thiomorpholinyl, piperidinyl and piperazinyl, particularly preferably morpholinyl and piperazinyl.

5-membered heteroaryl in the definition of the R⁵ radical represents an aromatic monocyclic radical having 5 ring atoms and up to 4 heteroatoms and/or hetero groups from the group consisting of S, O, N, SO and SO₂, where one nitrogen atom may also form an N-oxide, for example and with preference thienyl, furyl, pyrrolyl, thiazolyl, oxazolyl, isoxazolyl, oxadiazolyl, pyrazolyl, imidazolyl, triazolyl and tetrazolyl, particularly preferably triazolyl and tetrazolyl.

5-membered heterocycle in the definition of the R⁷ and R⁸ radicals represents a saturated, partially unsaturated or aromatic monocyclic radical having 5 ring atoms and up to 2 heteroatoms and/or hetero groups from the group consisting of S, O, N, SO and SO₂, where a nitrogen atom may also form an N-oxide. This 5-membered heterocycle together with the phenyl ring to which it is attached represents, for example and with preference, 2,3-dihydro-1-benzothiophen-5-yl, 1,3-dihydro-2-benzothiophen-5-yl, 2,3-dihydro-1-benzofuran-5-yl, 1,3-dihydro-2-benzofuran-5-yl, indolin-5-yl, isoindolin-5-yl, 2,3-dihydro-1H-indazol-5-yl, 2,3-dihydro-1H-benzimidazol-5-yl, 1,3-dihydro-2,1-benzoxazol-5-yl, 2,3-dihydro-1,3-benzoxazol-5-yl, 1,3-dihydro-2,1-benzothiazol-5-yl, 2,3-dihydro-1,3-benzothiazol-5-yl, 1H-benzimidazol-5-yl, 1H-indazol-5-yl, 1,2-benzoxazol-5-yl, indol-5-yl, isoindol-5-yl, benzofuran-5-yl, benzothiophen-5-yl, 2,3-dihydro-1-benzothiophen-6-yl, 1,3-dihydro-2-benzothiophen-6-yl, 2,3-dihydro-1-benzofuran-6-yl, 1,3-dihydro-2-benzofuran-6-yl, indolin-6-yl, isoindolin-6-yl, 2,3-dihydro-1H-indazol-6-yl, 2,3-dihydro-1H-benzimidazol-6-yl, 1,3-dihydro-2,1-benzoxazol-6-yl, 2,3-dihydro-1,3-benzoxazol-6-yl, 1,3-dihydro-2,1-benzothiazol-6-yl, 2,3-dihydro-1,3-benzothiazol-6-yl, 1H-benzimidazol-6-yl, 1H-indazol-6-yl, 1,2-benzoxazol-6-yl, indol-6-yl, isoindol-6-yl, benzofuran-6-yl and benzothiophen-6-yl, particularly preferably 1H-benzimidazol-6-yl and 2,3-dihydro-1H-indazol-6-yl, very particularly preferably 2,3-dihydro-1H-indazol-6-yl.

4- to 8-membered heterocyclyl attached via a carbon atom in the definition of the radical R¹⁰ represents a saturated or partially unsaturated monocyclic or bicyclic radical which is attached via a carbon atom and which has 4 to 8 ring atoms, preferably 5 or 6 ring atoms, and up to 3 heteroatoms and/or hetero groups, preferably 1 or 2 heteroatoms and/or hetero groups from the group consisting of S, O, N, SO and SO₂, where a nitrogen atom may also form an N-oxide, for example and with preference azetidinyl, pyrrolidinyl, piperidinyl, tetrahydropranyl, 3-azabicyclo[3.1.0]hex-6-yl, 8-azabicyclo[3.2.1]oct-3-yl and azepanyl, particularly preferably pyrrolidinyl and piperidinyl.

4- to 7-membered heterocycle in the definition of the R¹⁰ and R¹¹ radicals represents a saturated or partially unsaturated, monocyclic or bicyclic radical having 4 to 7 ring atoms, preferably 5 or 6 ring atoms, and up to 3 heteroatoms and/or hetero groups, preferably 1 or 2 heteroatoms and/or hetero groups, from the group of S, O, N, SO and SO₂, where one nitrogen atom may also form an N-oxide, for example and with preference azetidinyl, pyrrolidinyl, morpholinyl, thiomorpholinyl, piperidinyl, piperazinyl, 3-azabicyclo[3.1.0]hex-6-yl, 8-azabicyclo[3.2.1]oct-3-yl and azepanyl, particularly preferably pyrrolidinyl.

In the formulae of the group which may represent R¹, the end point of the line marked by # in each case does not represent a carbon atom or a CH₂ group, but is part of the bond to the atom to which R¹ is attached.

Preference is given to compounds of the formula (I) in which
R¹ represents a group of the formula

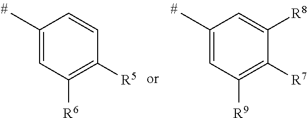

where # is the point of attachment to the nitrogen atom,
R⁵ represents 5-membered heteroaryl,
   where heteroaryl may be substituted by a substituent selected from the group consisting of oxo, chlorine and C₁-C₃-alkyl,
      in which alkyl may be substituted by 1 to 2 substituents independently of one another selected from the group consisting of hydroxycarbonyl and methoxy,
      or
      in which alkyl may be substituted by 1 to 7 fluorine substituents,
      or
      in which alkyl is substituted by a hydroxycarbonyl substituent and in which alkyl is additionally substituted by 1 to 6 fluorine substituents,
R⁶ represents hydrogen or fluorine,
R⁷ and R⁸ together with the carbon atoms to which they are attached form a 5-membered heterocycle,
   where the heterocycle may be substituted by 1 to 2 substituents independently of one another selected from the group consisting of oxo, chlorine, hydroxy, C₁-C₃-alkyl, pyrazolyl and pyridyl,
      in which alkyl may be substituted by 1 to 2 substituents independently of one another selected from the group consisting of hydroxycarbonyl and methoxy,
      or
      in which alkyl may be substituted by 1 to 7 fluorine substituents,
      or
      in which alkyl is substituted by a hydroxycarbonyl substituent and in which alkyl is additionally substituted by 1 to 6 fluorine substituents,
R⁹ represents hydrogen or fluorine,
R² represents hydrogen, fluorine, methyl or methoxy,
R³ represents hydrogen, fluorine, chlorine, C₁-C₄-alkyl, methoxy or trifluoromethyl,
R⁴ represents amino, cyano, C₁-C₃-alkoxy, C₁-C₃-alkylamino, C₁-C₃-alkoxycarbonyl, C₁-C₃-alkylcarbonylamino, C₁-C₃-alkylsulphonyl, —S(O)₂NR¹⁰R¹¹ or 5- to 7-membered heterocyclyl which is attached via a nitrogen atom,
   where alkoxy may be substituted by 1 to 2 substituents independently of one another selected from the group consisting of C₁-C₃-alkoxy,
   and
   where alkylcarbonylamino may be substituted by a —NH(CO)CH₂NH(CO)CH₂NH₂ substituent,
   and
   where heterocyclyl may be substituted by 1 to 2 substituents selected independently from the group consisting of oxo and C₁-C₄-alkyl,
   and
   where
   R¹⁰ represents hydrogen, C₁-C₃-alkyl, C₃-C₆-cycloalkyl or 4- to 8-membered heterocyclyl which is attached via a carbon atom,
      in which alkyl may be substituted by 1 to 2 hydroxy substituents,
      and
      in which heterocyclyl may be substituted by 1 to 2 substituents independently of one another selected from the group consisting of C₁-C₄-alkyl,
   R¹¹ represents hydrogen or C₁-C₃-alkyl,
   or
   R¹⁰ and R¹¹ together with the nitrogen atom to which they are attached form a 4- to 7-membered heterocycle,
      in which the heterocycle may be substituted by 1 to 2 substituents independently of one another selected from the group consisting of C₁-C₄-alkyl,

and the salts thereof, the solvates thereof and the solvates of the salts thereof.

Preference is also given to compounds of the formula (I) in which
R¹ represents a group of the formula

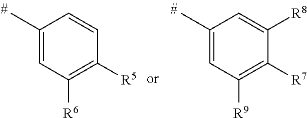

where # is the point of attachment to the nitrogen atom,
R⁵ represents 5-membered heteroaryl,
   where heteroaryl may be substituted by a substituent selected from the group consisting of oxo and chlorine,
R⁶ represents hydrogen
R⁷ and R⁸ together with the carbon atoms to which they are attached form a 5-membered heterocycle,
   where the heterocycle may be substituted by an oxo substituent,
R⁹ represents hydrogen,
R² represents hydrogen,
R³ represents hydrogen, fluorine, chlorine, methyl or trifluoromethyl,
R⁴ represents cyano, C₁-C₃-alkoxy, methoxycarbonyl, methylcarbonylamino, methylsulphonyl, —S(O)₂NR¹⁰R¹¹ or 5- to 7-membered heterocyclyl which is attached via a nitrogen atom,
   where alkoxy may be substituted by a methoxy substituent,
   and
   where methylcarbonylamino may be substituted by a —NH(CO)CH₂NH(CO)CH₂NH₂ substituent,
   and
   where heterocyclyl may be substituted by 1 to 2 substituents selected independently from the group consisting of oxo and methyl,
   and
   where
   R¹⁰ represents hydrogen, C₁-C₃-alkyl, cyclopropyl or 4- to 8-membered heterocyclyl which is attached via a carbon atom,
      in which alkyl may be substituted by 1 to 2 hydroxy substituents,
      and
      in which heterocyclyl may be substituted by 1 to 2 methyl substituents,
   R¹¹ represents hydrogen or C₁-C₃-alkyl,
   or
   R¹⁰ and R¹¹ together with the nitrogen atom to which they are attached form a 4- to 7-membered heterocycle,

and the salts thereof, the solvates thereof and the solvates of the salts thereof.

Preference is also given to compounds of the formula (I) in which
R¹ represents a group of the formula

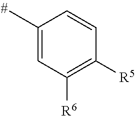

where # is the point of attachment to the nitrogen atom,
R⁵ is triazolyl or tetrazolyl,
   where triazolyl may be substituted by a substituent selected from the group consisting of oxo and chlorine,
R⁶ represents hydrogen
or
R¹ represents 2,3-dihydro-1H-indazol-6-yl,
   where 2,3-dihydro-1H-indazol-6-yl may be substituted by an oxo substituent,
R² represents hydrogen
R³ represents hydrogen, fluorine, chlorine, methyl or trifluoromethyl,
R⁴ represents —S(O)₂NR¹⁰R¹¹,
   where
   R¹⁰ represents hydrogen, C₁-C₃-alkyl, cyclopropyl or heterocyclyl which is attached via a carbon atom and selected from the group consisting of pyrrolidinyl and piperidinyl,
      in which alkyl may be substituted by 1 to 2 hydroxy substituents,
      and
      in which pyrrolidinyl and piperidinyl may be substituted by 1 to 2 methyl substituents,
   R¹¹ represents hydrogen or C₁-C₃-alkyl,
   or
   R¹⁰ and R¹¹ together with the nitrogen atom to which they are attached form a pyrrolidinyl,

and the salts thereof, the solvates thereof and the solvates of the salts thereof.

Preference is also given to compounds of the formula (I) in which

R¹ represents a group of the formula

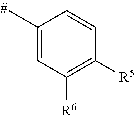

where # is the point of attachment to the nitrogen atom,
R⁵ represents triazolyl or tetrazolyl,
   where triazolyl may be substituted by a substituent selected from the group consisting of oxo and chlorine,
and
R⁶ represents hydrogen.

Preference is also given to compounds of the formula (I) in which
R¹ represents 2,3-dihydro-1H-indazol-6-yl,
   where 2,3-dihydro-1H-indazol-6-yl may be substituted by an oxo substituent.

Preference is also given to compounds of the formula (I) in which R² represents hydrogen.

Preference is also given to compounds of the formula (I) in which R³ represents hydrogen, fluorine, chlorine, methyl or trifluoromethyl.

Preference is also given to compounds of the formula (I) in which
R⁴ represents amino, cyano, C₁-C₃-alkoxy, C₁-C₃-alkylamino, C₁-C₃-alkoxycarbonyl, C₁-C₃-alkylcarbonylamino, C₁-C₃-alkylsulphonyl, —S(O)₂NR¹⁰R¹¹ or 5- to 7-membered heterocyclyl which is attached via a nitrogen atom,
   where alkoxy may be substituted by 1 to 2 substituents independently of one another selected from the group consisting of fluorine, hydroxy, amino, hydroxycarbonyl, C₁-C₃-alkoxy, C₁-C₃-alkylamino, difluoromethyl, trifluoromethyl, —(OCH₂CH₂)ₙ—OCH₃, —(OCH₂CH₂)ₘ—OH, morpholinyl, piperidinyl and pyrrolidinyl,
      in which n is a number from 1 to 6,
      in which m is a number from 1 to 6
   and
   where alkylcarbonylamino may be substituted by a —NH(CO)CH₂NH(CO)CH₂NH₂ substituent,
   and
   where heterocyclyl may be substituted by 1 to 2 substituents independently of one another selected from the group consisting of oxo, chlorine, cyano, hydroxy and C₁-C₄-alkyl,
   and
   where
   R¹⁰ represents hydrogen, C₁-C₃-alkyl, C₃-C₆-cycloalkyl, benzyl or 4- to 8-membered heterocyclyl which is attached via a carbon atom,
      in which alkyl may be substituted by 1 to 2 substituents independently of one another selected from the group consisting of hydroxy, amino, hydroxycarbonyl and methoxy,
      and
      in which heterocyclyl may be substituted by 1 to 2 substituents independently of one another selected from the group consisting of oxo, chlorine, cyano, hydroxy and C₁-C₄-alkyl,
   R¹¹ represents hydrogen or C₁-C₃-alkyl,
   or
   R¹⁰ and R¹¹ together with the nitrogen atom to which they are attached form a 4- to 7-membered heterocycle,
      in which the heterocycle may be substituted by 1 to 2 substituents independently of one another selected from the group consisting of oxo, fluorine, hydroxy, amino, hydroxycarbonyl, C₁-C₄-alkyl, C₁-C₃-alkylamino, difluoromethyl, trifluoromethyl, 2,2,2-trifluoroeth-1-yl, C₁-C₄-alkoxy carbonyl, aminocarbonyl and C₁-C₃-alkylaminocarbonyl.

Preference is also given to compounds of the formula (I) in which
R⁴ represents —S(O)₂NR¹⁰R¹¹,
   where
   R¹⁰ represents hydrogen, C₁-C₃-alkyl, cyclopropyl or heterocyclyl which is attached via a carbon atom and selected from the group consisting of pyrrolidinyl and piperidinyl,
      in which alkyl may be substituted by 1 to 2 hydroxy substituents,
      and
      in which pyrrolidinyl and piperidinyl may be substituted by 1 to 2 methyl substituents,
   R¹¹ represents hydrogen or C₁-C₃-alkyl,
   or
   R¹⁰ and R¹¹ together with the nitrogen atom to which they are attached form a pyrrolidinyl.

Irrespective of the particular combinations of the radicals specified, the individual radical definitions specified in the particular combinations or preferred combinations of radicals are also replaced as desired by radical definitions from other combinations.

Very particular preference is given to combinations of two or more of the abovementioned preferred ranges.

The invention further provides a process for preparing the compounds of the formula (I), or the salts thereof, solvates thereof and the solvates of the salts thereof, wherein the compounds of the formula

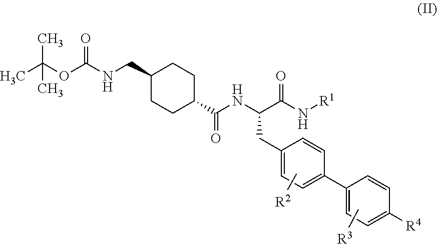

in which

R¹, R², R³ and R⁴ are each as defined above,

are reacted with an acid.

The reaction is generally effected in inert solvents, preferably within a temperature range from room temperature to 60° C. at standard pressure.

Inert solvents are, for example, halogenated hydrocarbons such as dichloromethane, trichloromethane, tetrachloromethane or 1,2-dichloroethane, or ethers such as tetrahydrofuran or dioxane, preference being given to dioxane.

Acids are, for example, trifluoroacetic acid or hydrogen chloride in dioxane, preference being given to hydrogen chloride in dioxane.

The compounds of the formula (II) are known or can be prepared by

[A] reacting compounds of the formula

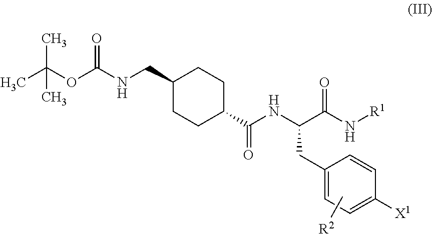

in which

R¹ and R² have the meaning given above, and

X¹ represents bromine or iodine,

with compounds of the formula

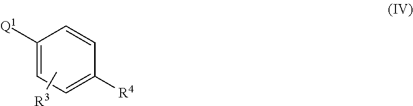

in which

R³ and R⁴ have the meaning given above, and

Q¹ represents —B(OH)₂, a boronic ester, preferably pinacol boronate, or —BF₃⁻K⁺,

under Suzuki coupling conditions,

or

[B] reacting compounds of the formula

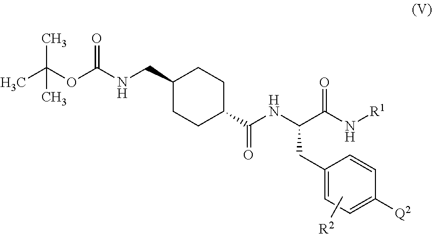

in which

R¹ and R² have the meaning given above, and

Q² represents —B(OH)₂, a boronic ester, preferably pinacol boronate, or —BF₃⁻K⁺,

with compounds of the formula

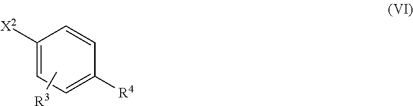

in which

R³ and R⁴ have the meaning given above, and

X² represents bromine or iodine,

under Suzuki coupling conditions,

or

[C] reacting compounds of the formula

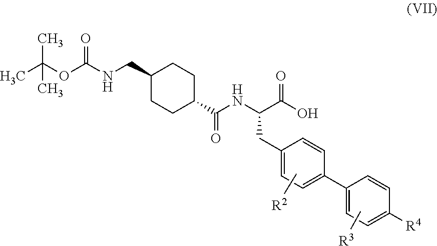

in which

R², R³ and R⁴ have the meaning given above

with compounds of the formula

H₂N—R¹ (VIII)

in which

R¹ has the meaning given above,

in the presence of a dehydrating reagent.

The reaction in process [A] is generally effected in inert solvents, in the presence of a catalyst, optionally in the presence of an additional reagent, optionally in a microwave, preferably within a temperature range from room temperature to 150° C. at standard pressure to 3 bar.

Catalysts are, for example, palladium catalysts customary for Suzuki reaction conditions, preference being given to catalysts such as dichlorobis(triphenylphosphine)palladium, tetrakistriphenylphosphinepalladium(0), palladium(II)acetate/triscyclohexylphosphine, tris(dibenzylideneacetone)dipalladium, bis(diphenylphosphaneferrocenyl)palladium(II)chloride, 1,3-bis(2,6-diisopropylphenyl)imidazol-2-ylidene(1,4-naphthoquinone)palladium dimer, allyl(chloro)(1,3-dimesityl-1,3-dihydro-2H-imidazol-2-ylidene)palladium, palladium(II)acetate/dicyclohexyl(2′,4′,6′-triisopropylbiphenyl-2-yl)phosphine, [1,1-bis(diphenylphosphino)ferrocene]palladium(II)chloride monodichloromethane adduct or XPhos precatalyst [(2′-aminobiphenyl-2-yl)(chloro)palladium dicyclohexyl(2′,4′,6′-triisopropylbiphenyl-2-yl)phosphane (1:1)], preference being given to tetrakistriphenylphosphinepalladium(0), [1,1-bis-(diphenylphosphino)ferrocene]palladium(II)chloride monodichloromethane adduct or XPhos precatalyst [(2′-aminobiphenyl-2-yl)(chloro)palladium dicyclohexyl(2′,4′,6′-triisopropylbiphenyl-2-yl)phosphane (1:1)].

Additional reagents are, for example, potassium acetate, caesium carbonate, potassium carbonate or sodium carbonate, potassium tert-butoxide, caesium fluoride or potassium phosphate, which may be present in aqueous solution; preferred additional reagents are those such as potassium acetate or a mixture of potassium acetate and sodium carbonate.

Inert solvents are, for example, ethers such as dioxane, tetrahydrofuran or 1,2-dimethoxyethane, hydrocarbons such as benzene, xylene or toluene, or carboxamides such as dimethylformamide or dimethylacetamide, alkyl sulphoxides such as dimethyl sulphoxide, or N-methylpyrrolidone or acetonitrile, or mixtures of the solvents with alcohols such as methanol or ethanol and/or water, preference being given to toluene, dimethylformamide or dimethyl sulphoxide.

The compounds of the formula (IV) are known, can be synthesized from the corresponding starting compounds by known processes or can be prepared analogously to the processes described in the Examples section.

The reaction in process [B] is effected as described for process [A].

The compounds of the formula (VI) are known, can be synthesized from the corresponding starting compounds by known processes or can be prepared analogously to the processes described in the Examples section.

The reaction in process [C] is generally effected in inert solvents, optionally in the presence of a base, preferably within a temperature range from 0° C. to the reflux of the solvents at standard pressure.

Suitable dehydrating agents here are, for example, carbodiimides such as N,N′-diethyl-, N,N′-dipropyl-, N,N′-diisopropyl-, N,N′-dicyclohexylcarbodiimide, N-(3-dimethylaminoisopropyl)-N′-ethylcarbodiimide hydrochloride (EDC) (optionally in the presence of pentafluorophenol (PFP)), N-cyclohexylcarbodiimide-N′-propyloxymethyl-polystyrene (PS-carbodiimide) or carbonyl compounds such as carbonyldiimidazole, or 1,2-oxazolium compounds such as 2-ethyl-5-phenyl-1,2-oxazolium 3-sulphate or 2-tert-butyl-5-methyl-isoxazolium perchlorate, or acylamino compounds such as 2-ethoxy-1-ethoxycarbonyl-1,2-dihydroquinoline, or propanephosphonic anhydride, or isobutyl chloroformate, or bis(2-oxo-3-oxazolidinyl)phosphoryl chloride or benzotriazolyloxytri(dimethylamino)phosphonium hexafluorophosphate, or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU), (benzotriazol-1-yloxy)bisdimethylaminomethylium fluoroborate (TBTU) or O-(7-azabenzotriazol-1-yl)-N,N,N′,N′-tetramethyluronium hexafluorophosphate (HATU), or 1-hydroxybenzotriazole (HOBt), or benzotriazol-1-yloxytris(dimethylamino)phosphonium hexafluorophosphate (BOP), or ethyl cyano(hydroxyimino)acetate (Oxyma), or (1-cyano-2-ethoxy-2-oxoethylideneaminooxy)dimethylaminomorpholinocarbenium hexafluorophosphate (COMU), or N-[(dimethylamino)(3H-[1,2,3]triazolo[4,5-b]pyridin-3-yloxy)methylidene]-N-methylmethanaminium hexafluorophosphate, or 2,4,6-tripropyl-1,3,5,2,4,6-trioxatriphosphinane 2,4,6-trioxide (T3P), or mixtures of these, with preference being given to N-[(dimethylamino)(3H-[1,2,3]triazolo[4,5-b]pyridin-3-yloxy)methylidene]-N-methylmethanaminium hexafluorophosphate or 2,4,6-tripropyl-1,3,5,2,4,6-trioxatriphosphinane 2,4,6-trioxide (T3P).

Bases are, for example, alkali metal carbonates such as sodium carbonate or potassium carbonate, or sodium bicarbonate or potassium bicarbonate, or organic bases such as trialkylamines, for example triethylamine, N-methylmorpholine, N-methylpiperidine, 4-dimethylaminopyridine or diisopropylethylamine; preference is given to diisopropylethylamine

Inert solvents are, for example, halogenated hydrocarbons such as dichloromethane or trichloromethane, hydrocarbons such as benzene, or other solvents such as nitromethane, tetrahydrofuran, dioxane, dimethylformamide, dimethyl sulphoxide, acetonitrile or pyridine, or mixtures of the solvents, preference being given to tetrahydrofuran or dimethylformamide or a mixture of dimethylformamide and pyridine.

The compounds of the formula (VIII) are known, can be synthesized from the corresponding starting compounds by known processes or can be prepared analogously to the processes described in the Examples section.

The compounds of the formula (III) are known or can be prepared by reacting compounds of the formula

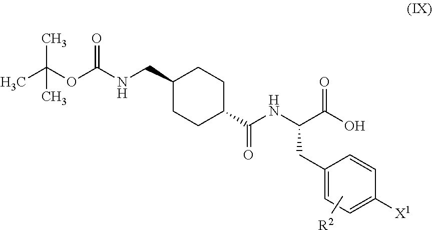

in which

R² has the meaning given above and

X¹ represents bromine or iodine,

with compounds of the formula (VIII) in the presence of a dehydrating reagent.

The reaction is carried out as described for process [C].

The compounds of the formula (IX) are known, can be synthesized from the corresponding starting compounds by known processes or can be prepared analogously to the processes described in the Examples section.

The compounds of the formula (V) are known or can be prepared by reacting compounds of the formula (III) with 4,4,4′,4′,5,5,5′,5′-octamethyl-2,2′-bi-1,3,2-dioxaborolane.

The reaction is generally effected in inert solvents, in the presence of a catalyst, optionally in the presence of an additional reagent, optionally in a microwave, preferably within a temperature range from room temperature to 150° C. at standard pressure to 3 bar. Hydrolysis in an acidic medium affords the corresponding boronic acids. Workup with potassium dihydrogenfluoride solution (KHF₂ solution) affords the corresponding trifluoroborates.

Catalysts are, for example, palladium catalysts customary for the borylation of aryl halides, preference being given to catalysts such as dichlorobis(triphenylphosphine)palladium, tetrakistriphenylphosphinepalladium(0), palladium(II)acetate/triscyclohexylphosphine, tris(dibenzylideneacetone)dipalladium, bis(diphenylphosphaneferrocenyl)palladium(II)chloride, 1,3-bis(2,6-diisopropylphenyl)imidazol-2-ylidene(1,4-naphthoquinone)palladium dimer, allyl(chloro)(1,3-dimesityl-1,3-dihydro-2H-imidazol-2-ylidene)palladium, palladium(II)acetate/dicyclohexyl(2′,4′,6′-triisopropylbiphenyl-2-yl)phosphine, [1,1-bis(diphenylphosphino)ferrocene]palladium(II)chloride monodichloromethane adduct or XPhos precatalyst [(2′-aminobiphenyl-2-yl)(chloro)palladium dicyclohexyl(2′,4′,6′-triisopropylbiphenyl-2-yl)phosphane (1:1)], preference being given to tetrakistriphenylphosphinepalladium(0) and [1,1-bis-(diphenylphosphino)ferrocene]palladium(II)chloride.

Additional reagents are, for example, potassium acetate, caesium carbonate, potassium carbonate or sodium carbonate, potassium tert-butoxide or sodium tert-butoxide, caesium fluoride, potassium phosphate or potassium phenoxide, preference being given to potassium acetate.

Inert solvents are, for example, ethers such as dioxane, tetrahydrofuran or 1,2-dimethoxyethane, hydrocarbons such as benzene, xylene or toluene, or carboxamides such as dimethylformamide or dimethylacetamide, alkyl sulphoxides such as dimethyl sulphoxide, or N-methylpyrrolidone or acetonitrile, preference being given to dioxane, dimethylformamide or dimethyl sulphoxide.

Literature: K. L. Billingslay, T. E. Barde, S. L Buchwald, Angew. Chem. 2007, 119, 5455 or T. Graening, Nachrichten aus der Chemie, January 2009, 57, 34.

The compounds of the formula (VII) are known or can be prepared by reacting compounds of the formula (IX) with compounds of the formula (IV) under Suzuki coupling conditions.

The reaction is effected as described for process [A].

The preparation of the starting compounds and of the compounds of the formula (I) can be illustrated by the synthesis scheme below.

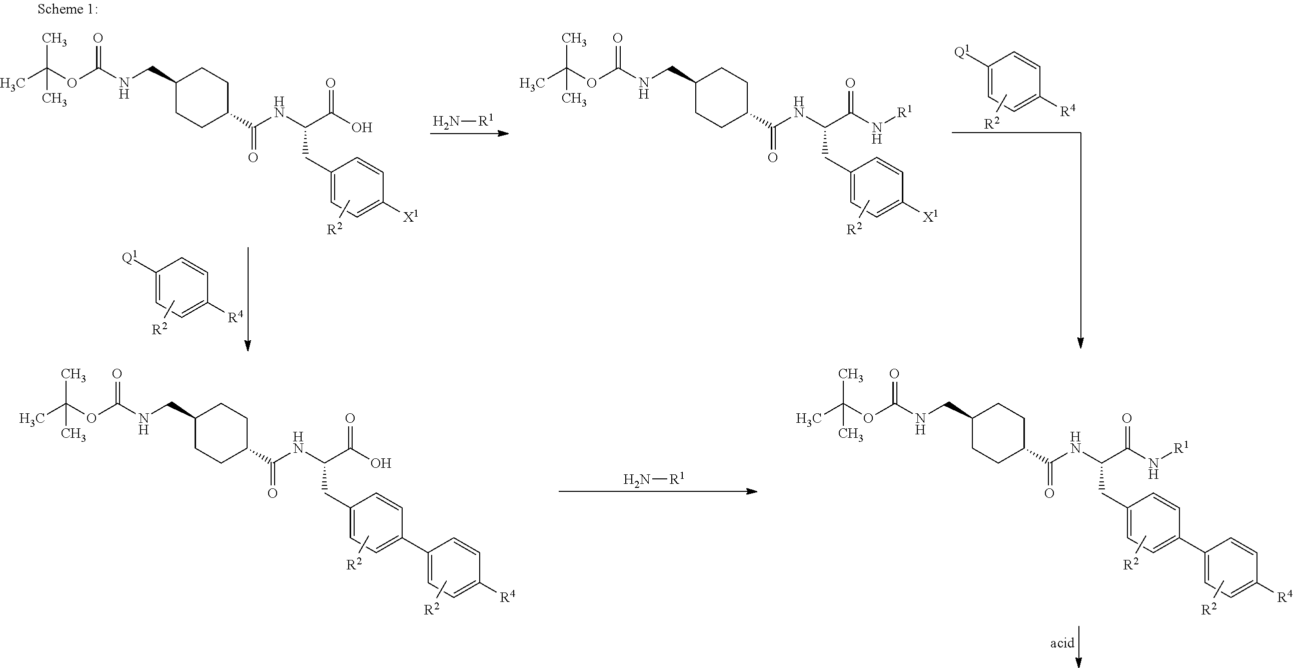

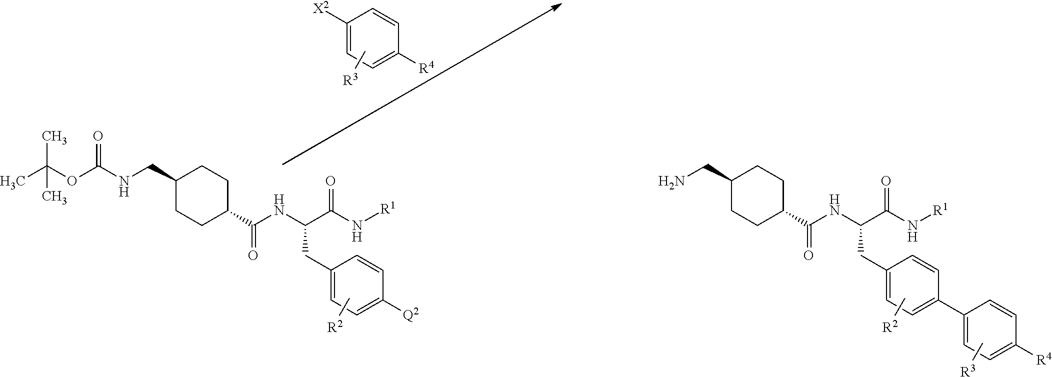

The compounds according to the invention have an unforeseeable useful pharmacological activity spectrum and good pharmacokinetic behaviour. They are compounds that influence the proteolytic activity of the serine proteases FXIa and kallikrein, and possibly plasmin. The inventive compounds inhibit the enzymatic cleavage of substrates that assume a major role in the activation of the blood coagulation cascade and platelet aggregation. If the inventive compounds inhibit plasmin activity, the result is inhibition of fibrinolysis.

They are therefore suitable for use as medicaments for treatment and/or prophylaxis of diseases in man and animals.

The present invention further provides for the use of the compounds according to the invention for the treatment and/or prophylaxis of disorders, in particular cardiovascular disorders, preferably thrombotic or thromboembolic disorders and/or thrombotic or thromboembolic complications.

“Thromboembolic disorders” in the sense of the present invention include in particular disorders such as acute coronary syndrome (ACS), ST-segment elevation myocardial infarction (STEMI) and non-ST-segment elevation myocardial infarction (non-STEMI), stable angina pectoris, unstable angina pectoris, reocclusions and restenoses after coronary interventions such as angioplasty, stent implantation or aortocoronary bypass, peripheral arterial occlusion diseases, pulmonary embolisms, venous thromboses, especially in deep leg veins and renal veins, transitory ischaemic attacks and also thrombotic and thromboembolic stroke.

The inventive compounds are therefore also suitable for the prevention and treatment of cardiogenic thromboembolisms, for example brain ischaemias, stroke and systemic thromboembolisms and ischaemias, in patients with acute, intermittent or persistent cardial arrhythmias, for example atrial fibrillation, and those undergoing cardioversion, and also in patients with heart valve disorders or with artificial heart valves.

In addition, the inventive compounds are suitable for the treatment and prevention of disseminated intravascular coagulation (DIC) which may occur in connection with sepsis inter alia, but also owing to surgical interventions, neoplastic disorders, burns or other injuries and may lead to severe organ damage through microthrombosis.

Thromboembolic complications are also encountered in microangiopathic haemolytic anaemias, extracorporeal circulatory systems, such as haemodialysis, and also prosthetic heart valves.

In addition, the inventive compounds are also used for influencing wound healing, for the prophylaxis and/or treatment of atherosclerotic vascular disorders and inflammatory disorders, such as rheumatic disorders of the locomotive system, coronary heart diseases, of heart failure, of hypertension, of inflammatory disorders, for example asthma, inflammatory pulmonary disorders, glomerulonephritis and inflammatory intestinal disorders, for example Crohn's disease or ulcerative colitis or acute renal failure, and additionally likewise for the prophylaxis and/or treatment of dementia disorders, for example Alzheimer's disease. In addition, the inventive compounds can be used for inhibiting tumour growth and the formation of metastases, for microangiopathies, age-related macular degeneration, diabetic retinopathy, diabetic nephropathy and other microvascular disorders, and also for the prevention and treatment of thromboembolic complications, for example venous thromboembolisms, for tumour patients, especially those undergoing major surgery or chemo- or radiotherapy.

In addition, the inventive compounds are also suitable for the prophylaxis and/or treatment of pulmonary hypertension.

The term “pulmonary hypertension” includes certain forms of pulmonary hypertension, as determined, for example, by the World Health Organization (WHO). Examples include pulmonary arterial hypertension, pulmonary hypertension associated with disorders of the left heart, pulmonary hypertension associated with pulmonary disorders and/or hypoxia and pulmonary hypertension owing to chronic thromboembolisms (CTEPH).

“Pulmonary arterial hypertension” includes idiopathic pulmonary arterial hypertension (IPAH, formerly also referred to as primary pulmonary hypertension), familial pulmonary arterial hypertension (FPAH) and associated pulmonary-arterial hypertension (APAH), which is associated with collagenoses, congenital systemic-pulmonary shunt vitia, portal hypertension, HIV infections, the ingestion of certain drugs and medicaments, with other disorders (thyroid disorders, glycogen storage disorders, Morbus Gaucher, hereditary teleangiectasia, haemoglobinopathies, myeloproliferative disorders, splenectomy), with disorders having a significant venous/capillary contribution, such as pulmonary-venoocclusive disorder and pulmonary-capillary haemangiomatosis, and also persisting pulmonary hypertension of neonatants.

Pulmonary hypertension associated with disorders of the left heart includes a diseased left atrium or ventricle and mitral or aorta valve defects.

Pulmonary hypertension associated with pulmonary disorders and/or hypoxia includes chronic obstructive pulmonary disorders, interstitial pulmonary disorder, sleep apnoea syndrome, alveolar hypoventilation, chronic high-altitude sickness and inherent defects.

Pulmonary hypertension owing to chronic thromboembolisms (CTEPH) comprises the thromboembolic occlusion of proximal pulmonary arteries, the thromboembolic occlusion of distal pulmonary arteries and non-thrombotic pulmonary embolisms (tumour, parasites, foreign bodies).

The present invention further provides for the use of the inventive compounds for production of medicaments for the treatment and/or prophylaxis of pulmonary hypertension associated with sarcoidosis, histiocytosis X and lymphangiomatosis.

In addition, the inventive substances may also be useful for the treatment of pulmonary and hepatic fibroses.

In addition, the inventive compounds may also be suitable for treatment and/or prophylaxis of disseminated intravascular coagulation in the context of an infectious disease, and/or of systemic inflammatory syndrome (SIRS), septic organ dysfunction, septic organ failure and multiorgan failure, acute respiratory distress syndrome (ARDS), acute lung injury (ALI), septic shock and/or septic organ failure.

In the course of an infection, there may be a generalized activation of the coagulation system (disseminated intravascular coagulation or consumption coagulopathy, hereinbelow referred to as “DIC”) with microthrombosis in various organs and secondary haemorrhagic complications. Moreover, there may be endothelial damage with increased permeability of the vessels and seeping of fluids and proteins into the extravasal lumen. As the infection progresses, there may be failure of an organ (for example kidney failure, liver failure, respiratory failure, central-nervous deficits and cardiovascular failure) or multiorgan failure.

In the case of DIC, there is a massive activation of the coagulation system at the surface of damaged endothelial cells, the surfaces of foreign bodies or injured extravascular tissue. As a consequence, there is coagulation in small vessels of various organs with hypoxia and subsequent organ dysfunction. This can be prevented by the inventive compounds. A secondary effect is the consumption of coagulation factors (for example factor X, prothrombin and fibrinogen) and platelets, which reduces the coagulability of the blood and may result in heavy bleeding.

In addition, the inventive compounds are also useful for the prophylaxis and/or treatment of hyperfibrinolysis. The prophylaxis and/or treatment may reduce or eliminate severe perioperative blood loss. Severe bleeding occurs in major operations, for example coronary artery bypass surgery, transplants or hysterectomy, and in the event of trauma, in the event of haemorrhagic shock or in the event of postpartum haemorrhage. In the aforementioned indications, there may be perioperative use of extracorporeal circulation systems or filter systems, for example heart and lung machines, haemofiltration, haemodialysis, extracorporeal membrane oxygenation or a ventricular support system, for example artificial heart. This additionally requires anticoagulation, for which the inventive compounds can also be used.

The inventive compounds are also suitable for anticoagulation during kidney replacement procedures, for example in the case of continuous veno-venous haemofiltration or intermittent haemodialysis.

In addition, the compounds according to the invention can also be used for preventing coagulation ex vivo, for example for preserving blood and plasma products, for cleaning/pretreating catheters and other medical auxiliaries and instruments, for coating synthetic surfaces of medical auxiliaries and instruments used in vivo or ex vivo or for biological samples which could contain factor XIa.

The present invention further provides for the use of the compounds according to the invention for the treatment and/or prophylaxis of disorders, especially the disorders mentioned above.

The present invention further provides for the use of the compounds according to the invention for production of a medicament for the treatment and/or prophylaxis of disorders, especially the disorders mentioned above.

The present invention further provides a method for the treatment and/or prophylaxis of disorders, especially the disorders mentioned above, using a therapeutically effective amount of a compound according to the invention.

The present invention further provides the compounds according to the invention for use in a method for the treatment and/or prophylaxis of disorders, especially the disorders mentioned above, using a therapeutically effective amount of a compound according to the invention.

The present invention further provides medicaments comprising a compound according to the invention and one or more further active compounds.

The present invention further provides a method for preventing the coagulation of blood in vitro, especially in banked blood or biological samples which could contain factor XIa, which is characterized in that an anticoagulatory amount of the inventive compound is added.

The present invention further provides medicaments comprising a compound according to the invention and one or more further active compounds, in particular for the treatment and/or prophylaxis of the disorders mentioned above. Preferred examples of active ingredients suitable for combinations include:
lipid-lowering substances, especially HMG-CoA (3-hydroxy-3-methylglutaryl-coenzyme A) reductase inhibitors, for example lovastatin (Mevacor), simvastatin (Zocor), pravastatin (Pravachol), fluvastatin (Lescol) and atorvastatin (Lipitor);
coronary therapeutics/vasodilatators, especially ACE (angiotensin converting enzyme) inhibitors, for example captopril, lisinopril, enalapril, ramipril, cilazapril, benazepril, fosinopril, quinapril and perindopril, or AII (angiotensin II) receptor antagonists, for example embusartan, losartan, valsartan, irbesartan, candesartan, eprosartan and temisartan, or β-adrenoceptor antagonists, for example carvedilol, alprenolol, bisoprolol, acebutolol, atenolol, betaxolol, carteolol, metoprolol, nadolol, penbutolol, pindolol, propanolol and timolol, or alpha-1-adrenoceptor antagonists, for example prazosine, bunazosine, doxazosine and terazosine, or diuretics, for example hydrochlorothiazide, furosemide, bumetanide, piretanide, torasemide, amiloride and dihydralazine, or calcium channel blockers, for example verapamil and diltiazem, or dihydropyridine derivatives, for example nifedipin (Adalat) and nitrendipine (Bayotensin), or nitro preparations, for example isosorbide 5-mononitrate, isosorbide dinitrate and glycerol trinitrate, or substances causing an increase in cyclic guanosine monophosphate (cGMP), for example stimulators of soluble guanylate cyclase, for example riociguat;
plasminogen activators (thrombolytics/fibrinolytics) and compounds which promote thrombolysis/fibrinolysis such as inhibitors of the plasminogen activator inhibitor (PAI inhibitors) or inhibitors of the thrombin-activated fibrinolysis inhibitor (TAFI inhibitors), for example tissue plasminogen activator (t-PA), streptokinase, reteplase and urokinase;
anticoagulatory substances (anticoagulants), for example heparin (UFH), low-molecular-weight heparins (LMW), for example tinzaparin, certoparin, parnaparin, nadroparin, ardeparin, enoxaparin, reviparin, dalteparin, danaparoid, semuloparin (AVE 5026), adomiparin (M118) and EP-42675/ORG42675;
direct thrombin inhibitors (DTI), for example Pradaxa (dabigatran), atecegatran (AZD-0837), DP-4088, SSR-182289A, argatroban, bivalirudin and tanogitran (BIBT-986 and prodrug BIBT-1011), hirudin;
direct factor Xa inhibitors, for example, rivaroxaban, apixaban, edoxaban (DU-176b), betrixaban (PRT-54021), R-1663, darexaban (YM-150), otamixaban (FXV-673/RPR-130673), letaxaban (TAK-442), razaxaban (DPC-906), DX-9065a, LY-517717, tanogitran (BIBT-986, prodrug: BIBT-1011), idraparinux and fondaparinux;
platelet aggregation-inhibiting substances (platelet aggregation inhibitors, thrombocyte aggregation inhibitors), for example acetylsalicylic acid (for example Aspirin), ticlopidine (Ticlid), clopidogrel (Plavix), prasugrel, ticagrelor, cangrelor, elinogrel, vorapaxar;
fibrinogen receptor antagonists (glycoprotein-IIb/IIIa antagonists), for example abciximab, eptifibatide, tirofiban, lamifiban, lefradafiban and fradafiban;
and also antiarrhythmics.
various antibiotics or antifungal medicaments, either as calculated therapy (prior to the presence of the microbial diagnosis) or as specific therapy;
vasopressors, for example norepinephrine, dopamine and vasopressin;
inotropic therapy, for example dobutamine;
corticosteroids, for example hydrocortisone and fludrocortisone;
recombinant human activated protein C, for example Xigris;
blood products, for example erythrocyte concentrates, thrombocyte concentrates, erythropietin and fresh frozen plasma.

“Combinations” for the purpose of the invention mean not only dosage forms which contain all the components (so-called fixed combinations) and combination packs which contain the components separate from one another, but also components which are administered simultaneously or sequentially, provided that they are used for prophylaxis and/or treatment of the same disease. It is likewise possible to combine two or more active ingredients with one another, meaning that they are thus each in two-component or multicomponent combinations.

The compounds of the invention can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The compounds of the invention can be administered in administration forms suitable for these administration routes.

Suitable administration forms for oral administration are those which function according to the prior art and deliver the inventive compounds rapidly and/or in modified fashion, and which contain the inventive compounds in crystalline and/or amorphized and/or dissolved form, for example tablets (uncoated or coated tablets, for example having enteric coatings or coatings which are insoluble or dissolve with a delay, which control the release of the compound according to the invention), tablets which disintegrate rapidly in the mouth, or films/wafers, films/lyophilisates, capsules (for example hard or soft gelatin capsules), sugar-coated tablets, granules, pellets, powders, emulsions, suspensions, aerosols or solutions.

Parenteral administration can be accomplished with avoidance of a resorption step (for example by an intravenous, intraarterial, intracardiac, intraspinal or intralumbar route) or with inclusion of a resorption (for example by an intramuscular, subcutaneous, intracutaneous, percutaneous or intraperitoneal route). Administration forms suitable for parenteral administration include preparations for injection and infusion in the form of solutions, suspensions, emulsions, lyophilizates or sterile powders.

Parenteral administration is preferred.

Suitable administration forms for the other administration routes are, for example, pharmaceutical forms for inhalation (including powder inhalers, nebulizers), nasal drops, solutions or sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (for example patches), milk, pastes, foams, dusting powders, implants or stents.

The compounds of the invention can be converted to the administration forms mentioned. This can be accomplished in a manner known per se by mixing with inert, nontoxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colorants (e.g. inorganic pigments, for example iron oxides) and flavour and/or odour correctants.

The present invention further provides medicaments comprising at least one inventive compound, preferably together with one or more inert nontoxic pharmaceutically suitable excipients, and the use thereof for the purposes mentioned above.

In the case of parenteral administration, it has generally been found to be advantageous to administer amounts of about 5 to 250 mg every 24 hours to achieve effective results. In the case of oral administration, the amount is about 5 to 500 mg every 24 hours.

In spite of this, it may be necessary, if appropriate, to deviate from the amounts specified, specifically depending on body weight, administration route, individual behaviour towards the active ingredient, type of formulation, and time or interval of administration.

Unless stated otherwise, the percentages in the tests and examples which follow are percentages by weight; parts are parts by weight. Solvent ratios, dilution ratios and concentration data for the liquid/liquid solutions are based in each case on volume. “w/v” means “weight/volume”. For example, “10% w/v” means: 100 ml of solution or suspension comprise 10 g of substance.

### A) EXAMPLES

Abbreviations:
bs/br. s. broad singlet (in NMR)
bd broad doublet (in NMR)
cat. catalytic
CI chemical ionization (in MS)
dd doublet of doublet (in NMR)
DMF dimethylformamide
DMSO dimethyl sulphoxide
dt doublet of triplet (in NMR)
EI electron impact ionization (in MS)
eq. equivalent(s)
ESI electrospray ionization (in MS)
h hour(s)
HATU O-(7-azabenzotriazol-1-yl)-N,N,N′,N′-tetramethyluronium hexafluorophosphate
HPLC high-pressure, high-performance liquid chromatography
LC-MS liquid chromatography-coupled mass spectrometry
m multiplet (in NMR)
M molar
min minute(s)
MS mass spectrometry
N normal
NMR nuclear magnetic resonance spectrometry
q quartet (in NMR)
quant. quantitative
quint quintet (in NMR)
RT room temperature
Rₜ retention time (in HPLC)
s singlet (in NMR)
TFA trifluoroacetic acid
THF tetrahydrofuran
UV ultraviolet spectrometry

HPLC and LC-MS Methods:

Method 1 (LC-MS): Instrument: Waters ACQUITY SQD UPLC system; column: Waters Acquity UPLC HSS T3 1.8μ 50 mm×1 mm; mobile phase A: 1 l of water+0.25 ml of 99% strength formic acid, mobile phase B: 1 l of acetonitrile+0.25 ml of 99% strength formic acid; gradient 0.0 min 90% A→1.2 min 5% A→2.0 min 5% A; oven: 50° C.; flow rate: 0.40 ml/min; UV detection: 210-400 nm.

Method 2 (LC-MS): Instrument: Micromass Quattro Premier with Waters UPLC Acquity; column: Thermo Hypersil GOLD 1.9μ 50 mm×1 mm; mobile phase A: 1 l of water+0.5 ml of 50% strength formic acid, mobile phase B: 1 l of acetonitrile+0.5 ml of 50% strength formic acid; gradient: 0.0 min 97% A→0.5 min 97% A→3.2 min 5% A→4.0 min 5% A; oven: 50° C.; flow rate: 0.3 ml/min; UV detection: 210 nm.

Method 3 (LC-MS): Instrument: Waters ACQUITY SQD UPLC system; column: Waters Acquity UPLC HSS T3 1.8μ 30 mm×2 mm; mobile phase A: 1 l of water+0.25 ml of 99% strength formic acid, mobile phase B: 1 l of acetonitrile+0.25 ml of 99% strength formic acid; gradient 0.0 min 90% A→1.2 min 5% A→2.0 min 5% A oven: 50° C.; flow rate: 0.60 ml/min; UV detection: 208-400 nm.

Method 4 (LC-MS): Instrument: Waters Acquity UPLC-MS SQD 3001; column: Acquity UPLC BEH C18 1.7μ 50 mm×2.1 mm; mobile phase A: water+0.1% formic acid, mobile phase B: acetonitrile; gradient: 0-1.6 min −99% B, 1.6-2.0 min 99% B; flow rate: 0.8 ml/min, temperature: 60° C.; injection: 2 μl; DAD scan: 210-400 nm; ELSD.

Method 5 (LC-MS): Instrument: Waters Acquity UPLC-MS SQD 3001; column: Acquity UPLC BEH C18 1.7μ 50 mm×2.1 mm; mobile phase A: water+0.2% ammonia, mobile phase B: acetonitrile; gradient: 0-1.6 min −99% B, 1.6-2.0 min 99% B; flow rate: 0.8 ml/min, temperature: 60° C.; injection: 2 μl; DAD scan: 210-400 nm; ELSD.

Method 6 (HPLC): System: Labomatic HD-3000 HPLC gradient pump, Labomatic Labocol Vario-2000 fraction collector; column: Chromatorex C-18 125 mm×30 mm, mobile phase A: 0.1% formic acid in water, mobile phase B: acetonitrile, gradient: A 95%/B 5%→A 55%/B 45%; flow rate: 150 ml/min; UV detection: 254 nm.

Method 7 (HPLC): System: Labomatic HD-3000 HPLC gradient pump, Labomatic Labocol Vario-2000 fraction collector; column: Chromatorex C-18 125 mm×30 mm, mobile phase A: 0.1% formic acid in water, mobile phase B: acetonitrile; gradient: A 90%/B 10%→A 50%/B 50%; flow rate: 150 ml/min; UV detection: 254 nm.

Method 8 (HPLC): System: Labomatic HD-3000 HPLC gradient pump, Labomatic Labocol Vario-2000 fraction collector; column: Chromatorex C-18 125 mm×30 mm, mobile phase A: 0.1% formic acid in water, mobile phase B: acetonitrile; gradient: A 85%/B 15%→A 45%/B 55%; flow rate: 150 ml/min; UV detection: 254 nm.

Method 9 (HPLC): System: Labomatic HD-3000 HPLC gradient pump, Labomatic Labocol Vario-2000 fraction collector; column: Chromatorex C-18 125 mm×30 mm, mobile phase A: 0.1% formic acid in water, mobile phase B: acetonitrile; gradient: A 80%/B 20%→A 40%/B 60%; flow rate: 150 ml/min; UV detection: 254 nm.

Method 10 (HPLC): Instrument: Waters autopurification system SQD; column: Waters XBridge C18 5μ 100 mm×30 mm; mobile phase A: water+0.1% formic acid (99% strength), mobile phase B: acetonitrile; gradient: 0-8.0 min 1-100% B, 8.0-10.0 min 100% B; flow rate 50.0 ml/min; temperature: RT; injection: 2500 μl; DAD scan: 210-400 nm.

Method 11 (HPLC): Instrument: Waters autopurification system SQD; column: Waters XBridge C18 5μ 100 mm×30 mm; mobile phase A: water+0.2% ammonia (32% strength), mobile phase B: acetonitrile; gradient: 0-8.0 min 1-100% B, 8.0-10.0 min 100% B; flow rate 50.0 ml/min; temperature: RT; injection: 2500 μl; DAD scan: 210-400 nm.

Method 12 (LC-MS): MS instrument: Waters (Micromass) QM; HPLC instrument: Agilent 1100 series; column: Agient ZORBAX Extend-C18 3.0 mm×50 mm 3.5 micron; mobile phase A: 1 l of water+0.01 mol of ammonium carbonate, mobile phase B: 1 l of acetonitrile; gradient 0.0 min 98% A→0.2 min 98% A→3.0 min 5% A→4.5 min 5% A; oven: 40° C.; flow rate: 1.75 ml/min; UV detection: 210 nm.

Method 13 (LC-MS): Instrument: Waters ACQUITY SQD UPLC system; column: Waters Acquity UPLC HSS T3 1.8μ 50 mm×1 mm; mobile phase A: 1 l of water+0.25 ml of 99% strength formic acid, mobile phase B: 1 l of acetonitrile+0.25 ml of 99% strength formic acid; gradient 0.0 min 95% A→6.0 min 5% A→7.5 min 5% A; oven: 50° C.; flow rate: 0.35 ml/min; UV detection: 210-400 nm.

Method 14 (LC-MS): MS instrument: Waters (Micromass) Quattro Micro; HPLC instrument: Agilent 1100 series; column: YMC-Triart C18 3μ 50×3 mm; mobile phase A: 1 l of water+0.01 mol of ammonium carbonate, mobile phase B: 1 l of acetonitrile; gradient: 0.0 min 10 0% A→2.75 min 5% A→4.5 min 5% A; oven: 40° C.; flow rate: 1.25 ml/min; UV detection: 210 nm.

Method 15 (HPLC): System: Labomatic HD-3000 HPLC gradient pump, Labomatic Labocol Vario-2000 fraction collector; column: Chromatorex C-18 125 mm×30 mm; mobile phase A: 0.1% ammonia in water, mobile phase B: acetonitrile, gradient: A 90%/B 10%→A 50%/B 50%; flow rate: 150 ml/min; UV detection: 254 nm.

Method 16 (HPLC): Isolera: Column: Cartridge SNAP 100 g, mobile phase: n-hexane/ethyl acetate 90%/10% to 100% ethyl acetate in 30 min, then 100% ethyl acetate to 85 min; flow rate: 40 ml/min; UV detection: 254 nm.

Microwave: The microwave reactor used was an instrument of the Biotage™ Initiator type.

When compounds according to the invention are purified by preparative HPLC by the above-described methods in which the eluents contain additives, for example trifluoroacetic acid, formic acid or ammonia, the compounds according to the invention may be obtained in salt form, for example as trifluoroacetate, formate or ammonium salt, if the compounds according to the invention contain a sufficiently basic or acidic functionality Such a salt can be converted to the corresponding free base or acid by various methods known to the person skilled in the art. Weaker salts can be converted to the corresponding chlorides by addition of a little hydrochloride.

In the case of the synthesis intermediates and working examples of the invention described hereinafter, any compound specified in the form of a salt of the corresponding base or acid is generally a salt of unknown exact stoichiometric composition, as obtained by the respective preparation and/or purification process. Unless specified in more detail, additions to names and structural formulae, such as “hydrochloride”, “trifluoroacetate”, “sodium salt” or “x HCl”, “x CF₃COOH”, “x Na⁺” should not therefore be understood in a stoichiometric sense in the case of such salts, but have merely descriptive character with regard to the salt-forming components present therein.

This applies correspondingly if synthesis intermediates or working examples or salts thereof were obtained in the form of solvates, for example hydrates, of unknown stoichiometric composition (if they are of a defined type) by the preparation and/or purification processes described.

If the starting compounds and examples contain an L-phenylalanine derivative as the central unit, the corresponding stereocentre is described as the (S) configuration. In the absence of further information, there was no check in individual cases as to whether partial epimerization of the stereocentre took place in the coupling of the L-phenylalanine intermediate with the amine H₂N—R¹. Thus, a mixture of the inventive compounds of (S) enantiomer and (R) enantiomer may be present. The main component is the (S) enantiomer depicted in each case.

Starting Materials

### Example 1A

### Methyl 4-bromo-N-[(trans-4-{[(tert-butoxycarbonyl)amino]methyl}cyclohexyl)carbonyl]-L-phenylalaninate

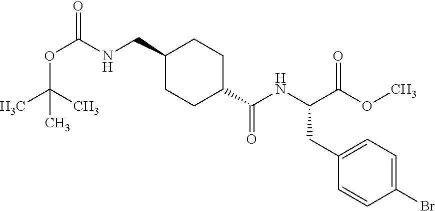

N,N-Diisopropylethylamine (381 ml, 2186 mmol) was added to a solution of methyl 4-bromo-L-phenylalaninate (250 g, 874 mmol) and trans-4-{[(tert-butoxycarbonyl)amino]methyl}cyclohexanecarboxylic acid (225 g, 874 mmol) in ethyl acetate (5012 ml). A 2,4,6-tripropyl-1,3,5,2,4,6-trioxatriphosphinane 2,4,6-trioxide solution (50% in dimethylformamide, 766 ml, 1312 mmol) was added dropwise, and the suspension was then stirred at RT for 3 h. The reaction mixture was then stirred into water and extracted three times with ethyl acetate. The organic phase was washed with saturated aqueous sodium hydrogencarbonate solution, saturated aqueous ammonium chloride solution, and saturated aqueous sodium chloride solution. The solution was dried over sodium sulphate and the solvent was removed. This gave 420 g (97% of theory) of the title compound.

¹H-NMR (400 MHz, DMSO-d₆): δ=0.68-0.92 (m, 2H), 1.04-1.32 (m, 4H), 1.37 (s, 9H), 1.48-1.73 (m, 4H), 2.03 (m, 1H), 2.74 (m, 2H), 2.78-2.90 (m, 1H), 2.94-3.05 (m, 1H), 4.36-4.50 (m, 1H), 6.72-6.85 (m, 1H), 7.17 (d, 2H), 7.46 (d, 2H), 8.15 (d, 1H).

LC-MS (Method 1): Rₜ=1.14 min; MS (ESIpos): m/z=497 [M+H]⁺.

### Example 2A

### 4-Bromo-N-[(trans-4-{[(tert-butoxycarbonyl)amino]methyl}cyclohexyl)carbonyl]-L-phenylalanine

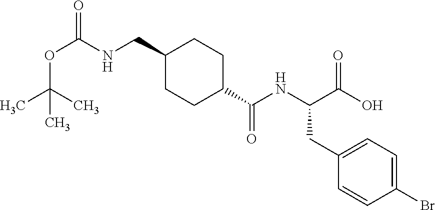

A solution of lithium hydroxide (72 g, 3015 mmol) in water (600 ml) was added to a solution of methyl 4-bromo-N-[(trans-4-{[(tert-butoxycarbonyl)amino]methyl}cyclohexyl)carbonyl]-L-phenylalaninate in tetrahydrofuran (3000 ml). The suspension was stirred at RT for 16 h. The reaction mixture was acidified with 1N hydrochloric acid solution, and ethyl acetate was added. The organic phase was washed with saturated aqueous sodium chloride solution and dried over sodium sulphate, and the solvent was removed. This gave 284 g (97% of theory) of the title compound.

¹H-NMR (400 MHz, DMSO-d₆): δ=0.71-0.90 (m, 2H), 1.22 (d, 4H), 1.37 (s, 9H), 1.45-1.73 (m, 5H), 2.03 (m, 1H), 2.67-2.88 (m, 3H), 2.95-3.09 (m, 1H), 4.38 (m, 1H), 6.77 (s, 1H), 7.17 (d, 2H), 7.46 (d, 2H), 7.99 (d, 1H), 12.65 (br. s, 1H).

LC-MS (Method 1): Rₜ=1.03 min; MS (ESIneg): m/z=481 [M−H]⁻.

### Example 3A

### 4-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide

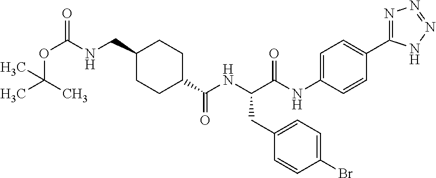

N,N-Diisopropylethylamine (9.6 ml, 55 mmol) was added to a solution of 4-bromo-N-[(trans-4-{[(tert-butoxycarbonyl)amino]methyl}cyclohexyl)carbonyl]-L-phenylalanine (11 mg, 22 mmol) and 4-(1H-tetrazol-5-yl)aniline (4 g, 24 mmol) in dimethylformamide (161 ml). At 0° C., a 2,4,6-tripropyl-1,3,5,2,4,6-trioxatriphosphinane 2,4,6-trioxide solution (50% in dimethylformamide, 16.9 g, 27 mmol) was added dropwise, and the suspension was then stirred at RT for 16 h. The reaction mixture was stirred into ethyl acetate (13 000 ml) and extracted three times with water (1570 ml each time). The organic phase was dried with sodium sulphate and the solvent was removed. The crude product was stirred with acetonitrile and filtered off with suction. This gave 11.4 g (78% of theory) of the title compound.

¹H-NMR (400 MHz, DMSO-d₆): δ=0.67-0.90 (m, 2H), 1.24 (m, 4H), 1.37 (s, 9H), 1.51-1.74 (m, 4H), 2.02-2.17 (m, 1H), 2.71-2.79 (m, 2H), 2.79-2.89 (m, 1H), 2.99-3.06 (m, 1H), 3.06-3.16 (m, 1H), 3.51-3.67 (m, 1H), 4.55-4.74 (m, 1H), 6.01-6.02 (m, 1H), 6.69-6.84 (m, 1H), 7.21-7.32 (m, 2H), 7.43-7.55 (m, 2H), 7.64-7.76 (m, 2H), 7.88-7.99 (m, 2H), 8.03-8.14 (m, 1H), 10.25 (s, 1H).

LC-MS (Method 1): Rₜ=1.07 min; MS (ESIneg): m/z=624 [M−H]⁻.

### Example 4A

### 4-Bromo-N-alpha-[(trans-4-{[(tert-butoxycarbonyl)amino]methyl}cyclohexyl)carbonyl]-N-[3-fluoro-4-(2H-tetrazol-5-yl)phenyl]-L-phenylalaninamide

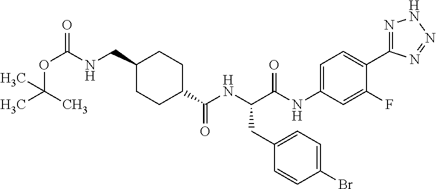

N,N-Diisopropylethylamine (10.8 ml, 62.1 mmol) was added to a solution of 4-bromo-N-[(trans-4-{[(tert-butoxycarbonyl)amino]methyl}cyclohexyl)carbonyl]-L-phenylalanine (10 mg, 20 7 mmol) and 3-fluoro-4-(2H-tetrazol-5-yl)aniline (4.1 g, 22.8 mmol) in ethyl acetate (210 ml). Subsequently, 2,4,6-tripropyl-1,3,5,2,4,6-trioxatriphosphinane 2,4,6-trioxide solution (50% in ethyl acetate, 32.9 g, 52 mmol) was added, and the reaction mixture was refluxed for 2 h and then stirred at RT for 48 h. Water was added to the reaction mixture, and the solid formed was filtered off with suction through a frit, washed with ethyl acetate and dried under reduced pressure. This gave 3.97 g (30% of theory) of the title compound.

¹H NMR (300 MHz, DMSO-d₆): δ=0.81 (m, 2H), 1.06-1.29 (m, 3H), 1.36 (s, 9H), 1.46-1.74 (m, 4H), 2.02-2.16 (m, 1H), 2.74 (m, 2H), 2.87 (dd, 1H), 3.00 (dd, 1H), 4.53-4.72 (m, 1H), 6.65-6.79 (m, 1H), 7.24 (d, 2H), 7.39-7.56 (m, 3H), 7.83 (dd, 1H), 8.00 (t, 1H), 8.15 (d, 1H), 10.61 (s, 1H).

LC-MS (Method 4): Rₜ=1.23 min; MS (ESIpos): m/z=645.3 [M+H]⁺.

### Example 5A

### Methyl N-[(trans-4-{[(tert-butoxycarbonyl)amino]methyl}cyclohexyl)carbonyl]-4-iodo-L-phenylalaninate

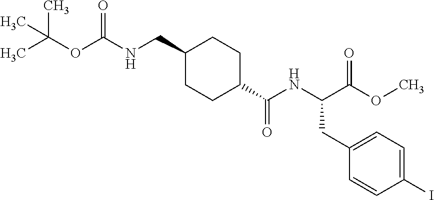

Methyl 4-iodo-L-phenylalaninate hydrochloride (5.7 g, 16.7 mmol), trans-4-{[(tert-butoxycarbonyl)amino]methyl}cyclohexanecarboxylic acid (4.4 g, 16.7 mmol) and N,N-diisopropylethylamine (11.7 ml, 67 mmol) were suspended in 90 ml of ethyl acetate. The solution was cooled to 0° C. Subsequently, 2,4,6-tripropyl-1,3,5,2,4,6-trioxatriphosphinane 2,4,6-trioxide (50% in ethyl acetate, 26.6 g, 42 mmol) was added dropwise, and the mixture was stirred at 0° C. for 30 minutes and at RT overnight. The mixture was quenched with water and extracted three times with ethyl acetate. The combined organic phases were washed once with saturated aqueous ammonium chloride solution and once with saturated aqueous sodium chloride solution, dried over magnesium sulphate, filtered and concentrated to dryness. The residue was recrystallized from acetonitrile. This gave 5.6 g (73% of theory) of the title compound.

¹H NMR (300 MHz, DMSO-d₆): δ ppm 0.68-0.86 (m, 2H), 1.02-1.27 (m, 3H), 1.33 (s, 9H), 1.45-1.55 (m, 1H), 1.62 (m, 3H), 1.92-2.04 (m, 1H), 2.70 (t, 2H), 2.79 (dd, 1H), 2.94 (dd, 1H), 3.56 (s, 3H), 4.27-4.44 (m, 1H), 6.69-6.79 (m, 1H), 6.98 (d, 2H), 7.59 (d, 2H), 8.10 (d, 1H).

LC-MS (Method 4): Rₜ=1.32 min; MS (ESIpos): m/z=545.2 [M+H]⁺.

### Example 6A

### N-[(trans-4-{[(tert-Butoxycarbonyl)amino]methyl}cyclohexyl)carbonyl]-4-iodo-L-phenylalanine

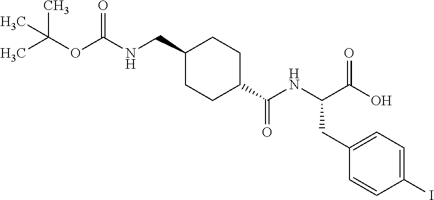

Methyl 4-iodo-N-[(trans-4-{[(tert-butoxycarbonyl)amino]methyl}cyclohexyl)carbonyl]-L-phenylalaninate (3.8 g, 7.0 mmol) was dissolved in 55 ml of tetrahydrofuran, the mixture was cooled to 0° C. and 5.3 ml of 2N aqueous sodium hydroxide solution were added. The mixture was allowed to come to RT and stirred at RT overnight. Subsequently, the tetrahydrofuran was drawn off and the aqueous phase was washed twice with tert-butyl methyl ether. The aqueous phase was then adjusted to pH 3 with 1N hydrochloric acid and the precipitated solid was filtered off. The aqueous phase was extracted three times with dichloromethane and the organic phase was concentrated. The residue from the organic phase was combined with the solid and dried under high vacuum. This gave 3.8 g (100% of theory) of the title compound.

¹H NMR (400 MHz, DMSO-d₆): δ ppm 0.72-0.85 (m, 2H), 1.08-1.27 (m, 3H), 1.33 (s, 9H), 1.63 (m, 4H), 1.87-1.96 (m, 1H), 2.70 (t, 2H), 2.83 (dd, 1H), 2.95 (dd, 1H), 3.83 (m, 1H), 6.69-6.75 (m, 1H), 6.84 (d, 2H), 6.93 (d, 1H), 7.47 (d, 2H).

LC-MS (Method 4): Rₜ=1.20 min; MS (ESIpos): m/z=531.1 [M+H]⁺.

### Example 7A

### N-alpha-[(trans-4-{[(tert-Butoxycarbonyl)amino]methyl}cyclohexyl)carbonyl]-4-iodo-N-[4-(1H-tetrazol-5-yl)phenyl]-L-phenylalaninamide

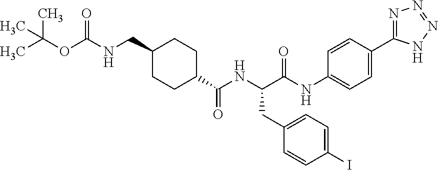

N-[(trans-4-{[(tert-Butoxycarbonyl)amino]methyl}cyclohexyl)carbonyl]-4-iodo-L-phenylalanine (1.9 g, 3.6 mmol), 4-(1H-tetrazol-5-yl)aniline (0.60 g, 3.6 mmol) and N,N-diisopropylethylamine (1.88 ml, 10.8 mmol) were suspended in 23 ml of ethyl acetate, and 2,4,6-tripropyl-1,3,5,2,4,6-trioxatriphosphinane 2,4,6-trioxide (50% in ethyl acetate, 5.73 g, 9.0 mmol) was added. The mixture was then heated under reflux for 3 h. Water and ethyl acetate were added to the reaction mixture, and the precipitated solid was filtered off with suction and dried under high vacuum. This gave 1.6 g (66% of theory) of the title compound.

¹H NMR (300 MHz, DMSO-d₆) δ ppm 0.66-0.95 (m, 2H), 1.22 (m, 2H), 1.33 (s, 9H), 1.38-1.56 (m, 3H), 1.57-1.70 (m, 2H), 1.95-2.13 (m, 1H), 2.65-2.86 (m, 2H), 2.90-3.00 (m, 1H), 3.04-3.15 (m, 1H), 3.54-3.62 (m, 1H), 4.50-4.69 (m, 1H), 6.63-6.78 (m, 1H), 7.07 (d, 2H), 7.60 (d, 2H), 7.68-7.77 (m, 2H), 7.93 (d, 2H), 8.00-8.09 (m, 1H), 10.27 (s, 1H).

LC-MS (Method 4): Rₜ=1.23 min; MS (ESIpos): m/z=673.6 [M+H]⁺.

### Example 8A

### N-alpha-[(trans-4-{[(tert-Butoxycarbonyl)amino]methyl}cyclohexyl)carbonyl]-N-[4-(3-chloro-4H-1,2,4-triazol-5-yl)phenyl]-4-iodo-L-phenylalaninamide

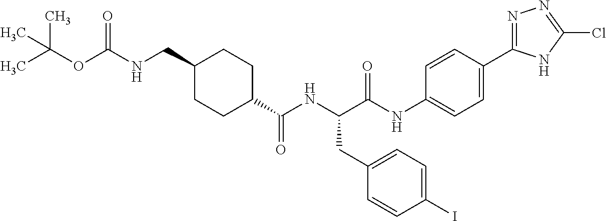

N-[(trans-4-{[(tert-Butoxycarbonyl)amino]methyl}cyclohexyl)carbonyl]-4-iodo-L-phenylalanine (2.4 g, 4.5 mmol), 4-(3-chloro-4H-1,2,4-triazol-5-yl)aniline (70%, 1.4 g, 5.0 mmol) and triethylamine (1.6 ml, 11 mmol) were suspended in 44 ml of ethyl acetate, and 2,4,6-tripropyl-1,3,5,2,4,6-trioxatriphosphinane 2,4,6-trioxide (50% in ethyl acetate, 5.3 ml, 9.0 mmol) was added. The mixture was then heated under reflux for 2 h and stirred at RT for a further 24 h. Water was added to the reaction mixture and the precipitated solid was filtered off with suction, washed with a little ethyl acetate and water and dried under high vacuum. This gave 2.5 g (78% of theory) of the title compound.

¹H NMR (400 MHz, DMSO-d₆): δ ppm 0.78 (m, 2H), 1.05-1.27 (m, 4H), 1.33 (s, 9H), 1.48-1.55 (m, 1H), 1.64 (m, 3H), 2.00-2.10 (m, 1H), 2.71 (t, 2H), 2.79 (dd, 1H), 2.95 (dd, 1H), 4.55-4.65 (m, 1H), 6.68-6.77 (m, 1H), 7.07 (d, 2H), 7.60 (d, 2H), 7.70 (d, 2H), 7.86 (d, 2H), 8.06 (d, 1H), 10.33 (s, 1H).

LC-MS (Method 4): Rₜ=1.31 min; MS (ESIpos): m/z=707.3 [M−H]⁺.

### Example 9A

### Methyl 4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]biphenyl-4-carboxylate

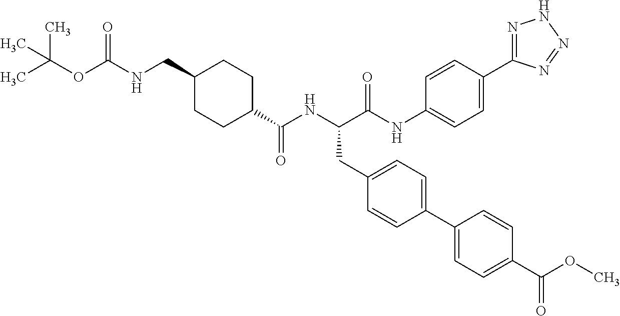

2000 mg (3.19 mmol) of 4-bromo-N-alpha-[(trans-4-{[(tert-butoxycarbonyl)amino]methyl}-cyclohexyl)carbonyl]-N-[4-(2H-tetrazol-5-yl)phenyl]-L-phenylalaninamide and 689 mg (3.81 mmol) of 4-methoxycarbonylphenylboronic acid were taken up in 32 ml of 1,2-dimethoxyethane. After the addition of 130 mg (0.16 mmol) of 1,1′-bis(diphenylphosphino)ferrocenepalladium(II)chloride and 3.2 ml of 2N aqueous sodium carbonate solution, the reaction mixture was stirred under reflux for 2 h and then concentrated. The residue was taken up in acetonitrile, boiled and hot-filtered through a Millipore syringe filter. After cooling to RT, the precipitate was filtered off with suction, washed with a little acetonitrile and dried under high vacuum. This gave 753 mg (34% of theory) of the title compound.

¹H-NMR (400 MHz, DMSO-d₆): δ=0.74-0.91 (m, 2H), 1.08-1.30 (m, 3H), 1.36 (s, 9H), 1.50-1.76 (m, 4H), 2.06-2.17 (m, 1H), 2.70-2.78 (m, 2H), 2.88-3.00 (m, 1H), 3.06-3.16 (m, 1H), 3.87 (s, 3H), 4.66-4.77 (m, 1H), 6.73-6.84 (m, 1H), 7.44 (d, 2H), 7.69 (d, 2H), 7.77-7.87 (m, 4H), 7.95-8.06 (m, 4H), 8.20 (d, 1H), 10.48 (s, 1H), 16.7 (br. s, 1H).

LC-MS (Method 1): Rₜ=1.11 min; MS (ESIneg): m/z=680 [M−H]⁻.

### Example 10A

### N-alpha-[(trans-4-{[(tert-Butoxycarbonyl)amino]methyl}cyclohexyl)carbonyl]-4-(4,4,5,5-tetramethyl-1,3,2-dioxaborolan-2-yl)-N-[4-(1H-tetrazol-5-yl)phenyl]-L-phenylalaninamide

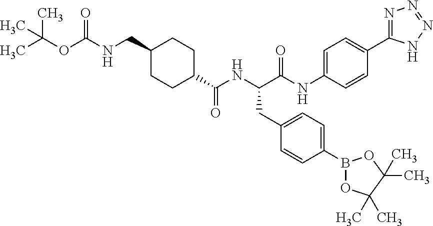

4-Bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide (4.1 g, 6.5 mmol) and bis(pinacolato)diborane (2.5 g, 9.8 mmol) were dissolved in 41 ml of DMSO, freed of air with argon and blanketed. 1,1′-Bis(diphenylphosphino)ferrocenedichloropalladium(II) (267 mg, 0.16 mmol) and potassium acetate (1.9 g, 19.6 mmol) were added and the reaction mixture was stirred at 110° C. for 24 h and at 150° C. in a microwave (Biotage Initiator) for 30 min and then converted further as the crude product.

LC-MS (Method 4): Rₜ=1.33 min; MS (ESIpos): m/z=674.6 [M−H]⁺.

### Example 11A

### Methyl 4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2-chlorobiphenyl-4-carboxylate

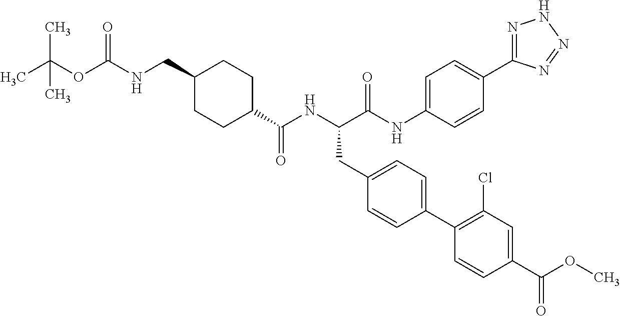

2N Aqueous sodium carbonate solution (8 ml) was added to a solution of 4-bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide (2350 mg, 3.75 mmol), 2-chloro-4-(methoxycarbonyl)phenylboronic acid (1608 mg, 7.5 mmol) and tetrakis(triphenylphosphine)palladium(0) (433 mg, 0.38 mmol) in 1,2-dimethoxyethane (20 ml) and ethanol (12 ml), and the mixture was heated at 100° C. for 4 h. This was followed by stirring at RT for 16 h. The reaction mixture was filtered through kieselguhr and the filtrate was separated by preparative HPLC (mobile phase: gradient of acetonitrile/water with 0.1% trifluoroacetic acid). The crude product was stirred with acetonitrile and filtered off with suction. This gave 1152 mg (36% of theory, 84% pure) of the title compound.

¹H-NMR (400 MHz, DMSO-d₆): δ=0.66-0.92 (m, 2H), 1.06-1.26 (m, 3H), 1.37 (s, 9H), 1.47-1.77 (m, 4H), 2.01-2.19 (m, 1H), 2.69-2.79 (m, 2H),2.86-3.02 (m, 1H), 3.06-3.22 (m, 1H), 3.89 (s, 3H), 4.64-4.85 (m, 1H), 6.71-6.91 (m, 1H), 7.42 (m, 5H), 7.78-7.88 (m, 2H), 7.93-8.10 (m, 4H), 8.14-8.34 (d, 1H), 10.44 (s, 1H), 16.73 (br. s, 1H).

LC-MS (Method 1): Rₜ=1.16 min; MS (ESIpos): m/z=716 [M+H]⁺.

### Example 12A

### 4-Bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide

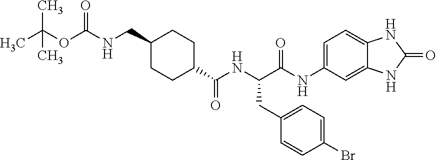

N,N-Diisopropylethylamine (1.4 ml, 7 8 mmol) was added to a solution of 4-bromo-N-[(trans-4-{[(tert-butoxycarbonyl)amino]methyl}cyclohexyl)carbonyl]-L-phenylalanine (1500 mg, 3 mmol) and 5-amino-1,3-dihydro-2H-benzimidazol-2-one (555 mg, 24 mmol) in ethyl acetate (21 ml). A 2,4,6-tripropyl-1,3,5,2,4,6-trioxatriphosphinane 2,4,6-trioxide solution (50% in dimethylformamide, 2.2 ml, 3.7 mmol) and, until dissolution, dimethylformamide were added, and the mixture was then stirred at RT for 16 h. The reaction mixture was stirred into ethyl acetate, and washed twice with water and once with aqueous sodium chloride solution. The organic phase was dried with sodium sulphate and the solvent was removed. The crude product was stirred with acetonitrile and filtered off with suction. The residue was separated twice by preparative HPLC (mobile phase: gradient of acetonitrile/water with 0.1% trifluoroacetic acid). The crude product was stirred with methanol and filtered off with suction. This gave 202 mg (11% of theory) of the title compound.

¹H-NMR (400 MHz, DMSO-d₆): δ=0.69-0.89 (m, 2H), 1.04-1.29 (m, 3H), 1.37 (s, 9H), 1.67 (m, 4H), 2.04-2.17 (m, 1H), 2.75 (m, 3H), 2.94-3.07 (m, 1H), 4.54-4.75 (m, 1H), 6.68-6.83 (m, 1H), 6.96 (dd, 1H), 7.25 (d, 2H), 7.39-7.56 (m, 3H), 7.84 (s, 1H), 8.09 (d, 1H), 10.20 (s, 1H), 11.08 (br. s, 1H).

LC-MS (Method 1): Rₜ=1.00 min; MS (ESIpos): m/z=614 [M+H]⁺.

### Example 13A

### 4-Bromo-N-alpha-[(trans-4-{[(tert-butoxycarbonyl)amino]methyl}cyclohexyl)carbonyl]-N-[4-(3-chloro-4H-1,2,4-triazol-5-yl)phenyl]-L-phenylalaninamide

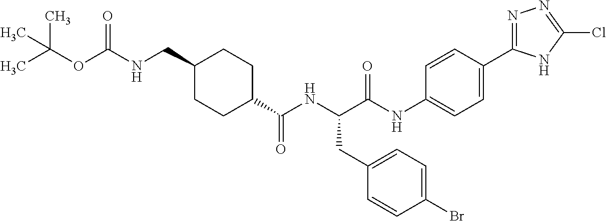

4-Bromo-N-[(trans-4-{[(tert-butoxycarbonyl)amino]methyl}cyclohexyl)carbonyl]-L-phenylalanine (6.3 g, 13 mmol), 4-(3-chloro-4H-1,2,4-triazol-5-yl)aniline (2.8 g, 14 3 mmol) and N,N-diisopropylamine (6.8 ml, 39 mmol) were suspended in 130 ml of ethyl acetate, and 2,4,6-tripropyl-1,3,5,2,4,6-trioxatriphosphinane 2,4,6-trioxide (50% in ethyl acetate, 21 g, 32.6 mmol) was added. The mixture was then heated under reflux for 3 h. Water was added to the reaction mixture, and the phases were separated. The aqueous phase was extracted three times with ethyl acetate. The combined organic phases were washed with saturated aqueous ammonium chloride solution and saturated aqueous sodium chloride solution, dried over sodium sulphate, filtered and concentrated. The residue was purified chromatographically by Isolera (Method 16) and HPLC (Method 11; 40-70% B). This gave 1.8 g (23% of theory) of the title compound.

¹H NMR (400 MHz, DMSO-d₆): δ=ppm 0.71-0.86 (m, 2H), 1.03-1.26 (m, 3H), 1.33 (s, 9H), 1.48-1.55 (m, 1H), 1.58-1.69 (m, 3H), 2.02-2.10 (m, 1H), 2.68-2.74 (m, 2H), 2.81 (dd, 1H), 2.98 (dd, 1H), 4.50-4.68 (m, 1H), 6.67-6.76 (m, 1H), 7.22 (d, 2H), 7.44 (d, 2H), 7.71 (d, 2H), 7.86 (d, 2H), 8.07 (d, 1H), 10.33 (s, 1H).

LC-MS (Method 4): Rₜ=1.28 min; MS (ESIneg): m/z=659.4 [M−H]⁻.

### Example 14A

### tert-Butyl 4-[({4′-[(2S)-2-{[(trans-4-{[(tert-butoxycarbonyl)amino]methyl}cyclohexyl)carbonyl]amino}-3-oxo-3-{[4-(1H-tetrazol-5-yl)phenyl]amino}propyl]-2-methylbiphenyl-4-yl}sulphonyl)amino]piperidine-1-carboxylate

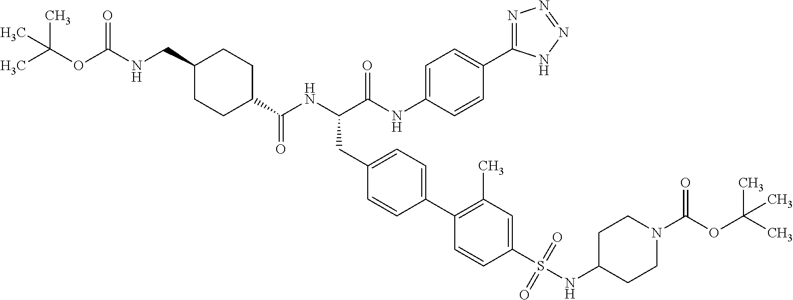

N-alpha-[(trans-4-{[(tert-Butoxycarbonyl)amino]methyl}cyclohexyl)carbonyl]-4-(4,4,5,5-tetramethyl-1,3,2-dioxaborolan-2-yl)-N-[4-(1H-tetrazol-5-yl)phenyl]-L-phenylalaninamide (200 mg, 0.3 mmol, crude product) and tert-butyl 4-{[(4-bromo-3-methylphenyl)sulphonyl]amino}piperidine-1-carboxylate (154 mg, 0.36 mmol) were dissolved in dimethyl sulphoxide (2 ml), and tetrakis(triphenylphosphine)palladium(0) (34 mg, 30 μmol), sodium carbonate (157 mg, 1.5 mmol) and water (0.45 ml, 25 mmol) were added. The reaction mixture was stirred at 110° C. in a microwave (Biotage Initiator) for 300 min, cooled, filtered and purified by chromatography via HPLC (Method 11). This gave 49 mg (18% of theory) of the title compound.

LC-MS (Method 5): Rₜ=0.93 min; MS (ESIpos): m/z=900.6 [M+H]⁺.

### Example 15A

### tert-Butyl [(trans-4-{[(2S)-3-(4′-acetamido-3′-fluorobiphenyl-4-yl)-1-oxo-1-{[4-(2H-tetrazol-5-yl)phenyl]amino}propan-2-yl]carbamoyl}cyclohexyl)methyl]carbamate

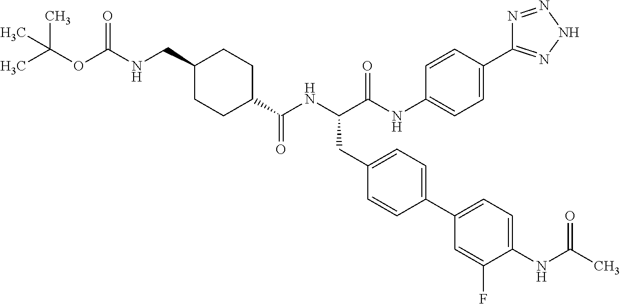

27.7 mg (23.9 μmol) of tetrakis(triphenylphosphine)palladium(0), 76.1 mg (0.72 mmol) of sodium carbonate and 0.36 ml (20.0 mmol) of water were added to a solution of 150 mg (0.24 mmol) of 4-bromo-N-alpha-[(trans-4-{[(tert-butoxycarbonyl)amino]methyl}cyclohexyl)carbonyl]-N-[4-(1H-tetrazol-5-yl)phenyl]-L-phenylalaninamide and 100.2 mg (0.36 mmol) of N-[2-fluoro-4-(4,4,5,5-tetramethyl-1,3,2-dioxaborolan-2-yl)phenyl]acetamide in 1.8 ml of DMSO. The mixture was treated in the microwave at 110° C. for 2 min. The mixture was filtered and purified by preparative HPLC (Method 8). This gave 73 mg (44% of theory) of the title compound.

LC-MS (Method 4): Rₜ=1.13 min; MS (ESIpos): m/z=699 [M+H]⁺.

### Example 16A

### tert-Butyl [(trans-4-{[(2S)-1-oxo-3-[4′-(3-oxomorpholin-4-yl)biphenyl-4-yl]-1-{[4-(2H-tetrazol-5-yl)phenyl]amino}propan- 2-yl]carbamoyl}cyclohexyl)methyl]carbamate

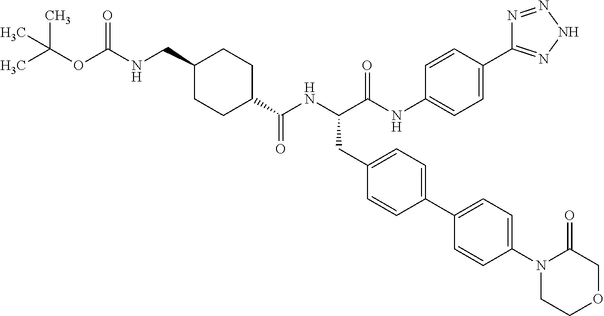

34.3 mg (29.7 μmol) of tetrakis(triphenylphosphine)palladium(0), 157.3 mg (1 5 mmol) of sodium carbonate and 0.45 ml (24.8 mmol) of water were added to a solution of 91.2 mg (0.35 mmol) of 4-(4-bromophenyl)morpholin-3-one and 200 mg (0.30 mmol) of N-alpha-[(trans-4-{[(tert-butoxycarbonyl)amino]methyl}cyclohexyl)carbonyl]-4-(4,4,5,5-tetramethyl-1,3,2-dioxaborolan-2-yl)-N-[4-(1H-tetrazol-5-yl)phenyl]-L-phenylalaninamide in 2 ml of DMSO. The mixture was treated in the microwave at 110° C. for 2.5 h. A little more tetrakis(triphenylphosphine)palladium(0) was then added and the mixture was stirred at 110° C. in a microwave for 60 min. The mixture was filtered and purified by preparative HPLC (Method 11). This gave 21 mg (10% of theory) of the title compound.

LC-MS (Method 5): Rₜ=0.79 min; MS (ESIpos): m/z=723.5 [M+H]⁺.

### Example 17A

### tert-Butyl [(trans-4-{[(2S)-3-[4′-(2-methoxyethoxy)biphenyl-4-yl]-1-oxo-1-{[4-(2H-tetrazol-5-yl)phenyl]amino}propan-2-yl]carbamoyl}cyclohexyl)methyl]carbamate

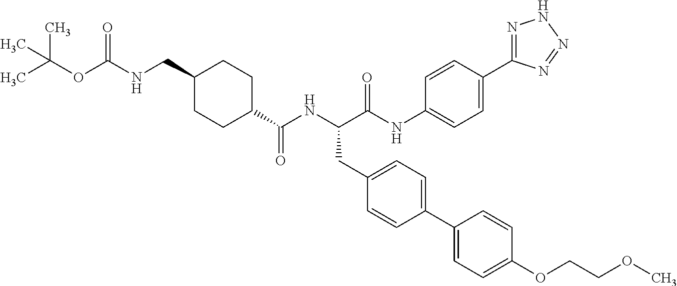

100 mg (0.16 mmol) of 4-bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide, 18.4 mg (0.02 mmol) of tetrakis(triphenylphosphine)palladium(0) and 78 mg (0.40 mmol) of [4-(2-methoxyethoxy)phenyl]boronic acid were taken up in 1.5 ml of 1,2-dimethoxyethane and 0.50 ml of ethanol. After the addition of 1.2 ml of 2N aqueous sodium carbonate solution, the mixture was stirred under reflux for 3 h and at RT for 16 h. The salts were removed by filtration of the reaction mixture and the filtrate was separated by preparative HPLC (mobile phase: gradient of acetonitrile/water with 0.1% trifluoroacetic acid). This gave 83 mg (75% of theory) of the title compound.

LC-MS (Method 1): Rₜ=1.10 min; MS (ESIneg): m/z=696 [M−H]⁻.

### Example 18A

### tert-Butyl [(trans-4-{[(2S)-3-[4′-(4-methylpiperazin-1-yl)biphenyl-4-yl]-1-oxo-1-{[4-(2H-tetrazol-5-yl)phenyl]amino}propan-2-yl]carbamoyl}cyclohexyl)methyl]carbamate trifluoroacetate

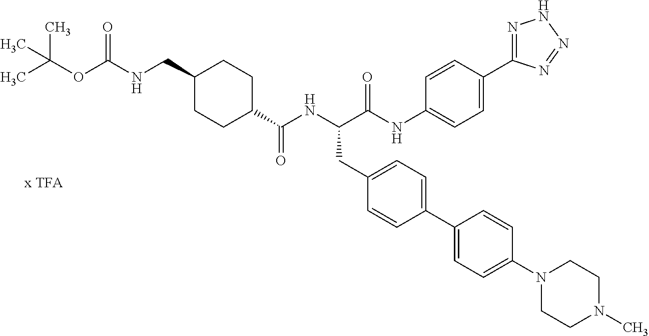

100 mg (0.16 mmol) of 4-bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide, 18.4 mg (0.02 mmol) of tetrakis(triphenylphosphine)palladium(0) and 113 mg (0.40 mmol) of potassium 4-(1-methyl-4-piperazinyl)phenyltrifluoroborate were taken up in 1.5 ml of 1,2-dimethoxyethane and 0.50 ml of ethanol. After the addition of 1.2 ml of 2N aqueous sodium carbonate solution, the mixture was stirred under reflux for 3 h and at RT for 16 h. The salts were removed by filtration of the reaction mixture and the filtrate was separated by preparative HPLC (mobile phase: gradient of acetonitrile/water with 0.1% trifluoroacetic acid). This gave 111 mg of a mixture of the title compound and the corresponding deprotected title compound, which was used directly in the next stage.

LC-MS (Method 1): Rₜ=0.79 min; MS (ESIneg): m/z=720 [M−H-TFA]⁻.

### Example 19A

### tert-Butyl 4-{4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]biphenyl-4-yl}piperazine-1-carboxylate

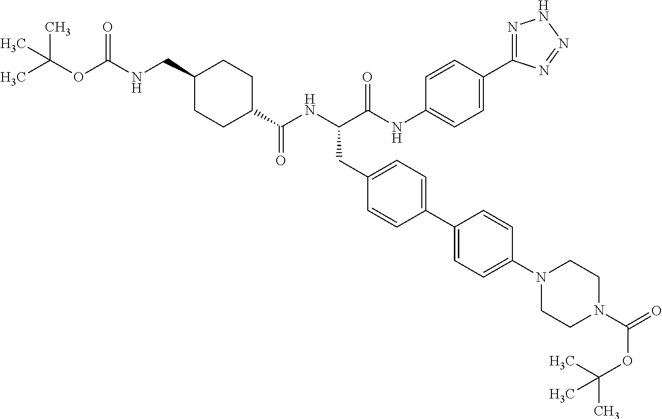

100 mg (0.16 mmol) of 4-bromo-N-alpha-[(trans-4-{[(tert-butoxycarbonyl)amino]methyl}-cyclohexyl)carbonyl]-N-[4-(2H-tetrazol-5-yl)phenyl]-L-phenylalaninamide and 18 mg (0.02 mmol) of tetrakis(triphenylphosphine)palladium(0) were taken up in 1.4 ml of 1,2-dimethoxyethane under argon and stirred at RT for 10 min A solution of 207 mg (0.48 mmol) of tert-butyl 4-[4-(4,4,5,5-tetramethyl-1,3,2-dioxaborolan-2-yl)phenyl]piperazine-1-carboxylate in 0.5 ml of ethanol was added dropwise to the reaction mixture, which was stirred at RT for a further 10 min. After the addition of 1.2 ml of 2N aqueous sodium carbonate solution, the mixture was stirred at RT for 5 min and under reflux for 3 h. A little methanol was added and the reaction mixture filtered through a Millipore syringe filter and separated by preparative HPLC (mobile phase: gradient of acetonitrile/water with 0.1% trifluoroacetic acid). This gave 93 mg (61% of theory) of the title compound.

LC-MS (Method 1): Rₜ=1.27 min; MS (ESIneg): m/z=806 [M−H]⁻.

### Example 20A

### tert-Butyl [(trans-4-{[(2S)-3-[4′-(morpholin-4-yl)biphenyl-4-yl]-1-oxo-1-{[4-(2H-tetrazol-5-yl)phenyl]amino}propan-2-yl]carbamoyl}cyclohexyl)methyl]carbamate

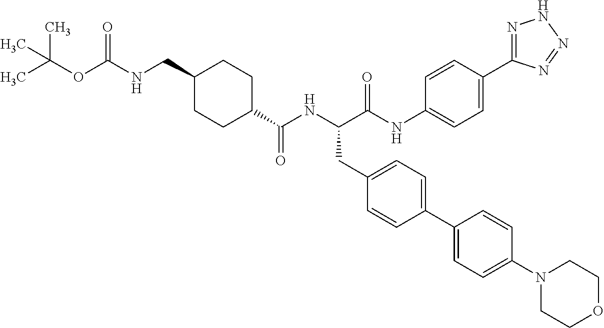

100 mg (0.16 mmol) of 4-bromo-N-alpha-[(trans-4-{[(tert-butoxycarbonyl)amino]methyl}-cyclohexyl)carbonyl]-N-[4-(2H-tetrazol-5-yl)phenyl]-L-phenylalaninamide and 18 mg (0.02 mmol) of tetrakis(triphenylphosphine)palladium(0) were taken up in 1.4 ml of 1,2-dimethoxyethane under argon and stirred at RT for 10 min A solution of 99 mg (0.48 mmol) of 4-(4-morpholinyl)phenylboronic acid in 0.5 ml of ethanol was added dropwise to the reaction mixture and stirred at RT for a further 10 min. After the addition of 1.2 ml of 2N aqueous sodium carbonate solution, the mixture was stirred at RT for 5 min and under reflux for 3 h. A little methanol was added and the reaction mixture filtered through a Millipore syringe filter and separated by preparative HPLC (mobile phase: gradient of acetonitrile/water with 0.1% trifluoroacetic acid). This gave 60 mg (44% of theory) of the title compound.

LC-MS (Method 1): Rₜ=1.11 min; MS (ESIneg): m/z=707 [M−H]⁻.

### Example 21A

### tert-Butyl 4-{4′-[(2S)-2-{[(trans-4-{[(tert-butoxycarbonyl)amino]methyl}cyclohexyl)carbonyl]amino}-3-oxo-3-{[4-(5-oxo-4,5-dihydro-1,2,4-oxadiazol-3-yl)phenyl]amino}propyl]biphenyl-4-yl}piperazine-1-carboxylate

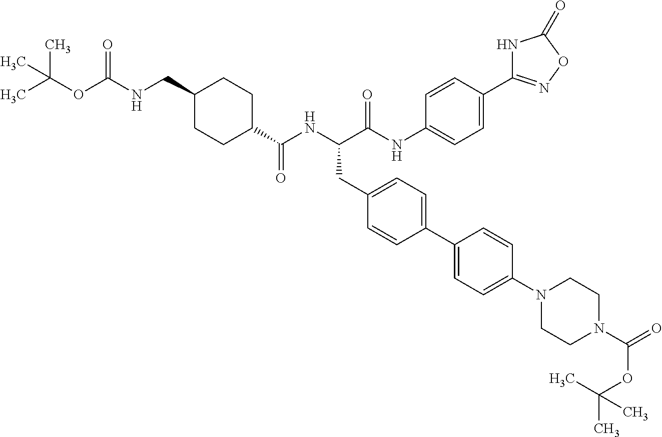

26 mg (0.04 mmol) of 4-bromo-N-alpha-[(trans-4-{[(tert-butoxycarbonyl)amino]methyl}cyclohexyl)carbonyl]-N-[4-(5-oxo-4,5-dihydro-1,2,4-oxadiazol-3-yl)phenyl]-L-phenylalaninamide and 5 mg (0.004 mmol) of tetrakis(triphenylphosphine)palladium(0) were taken up in 0.7 ml of 1,2-dimethoxyethane under argon and stirred at RT for 10 min A solution of 47 mg (0.12 mmol) of tert-butyl 4-[4-(4,4,5,5-tetramethyl-1,3,2-dioxaborolan-2-yl)phenyl]piperazine-1-carboxylate in 0.3 ml of ethanol was added dropwise to the reaction mixture, which was stirred at RT for a further 10 min. After the addition of 0.6 ml of 2N aqueous sodium carbonate solution, the mixture was stirred at RT for 5 min and under reflux for 3 h. A little methanol was added and the reaction mixture filtered through a Millipore syringe filter and separated by preparative HPLC (mobile phase: gradient of acetonitrile/water with 0.1% trifluoroacetic acid). This gave 20 mg (50% of theory) of the title compound.

LC-MS (Method 1): Rₜ=1.30 min; MS (ESIneg): m/z=822 [M−H]⁻.

### Example 22A

### tert-Butyl [(trans-4-{[(2S)-3-(4′-cyano-2′-methylbiphenyl-4-yl)-1-oxo-1-{[4-(2H-tetrazol-5-yl)phenyl]amino}propan-2-yl]carbamoyl}cyclohexyl)methyl]carbamate

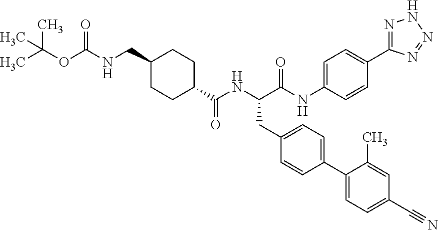 [SMILES: Cc1cc(C#N)ccc1-c1ccc(C[C@H](NC(=O)[C@H]2CC[C@H](CNC(=O)OC(C)(C)C)CC2)C(=O)Nc2ccc(-c3nn[nH]n3)cc2)cc1]

0.33 ml (0.67 mmol) of a 2M sodium carbonate solution in water was added to a solution of 140 mg (0.22 mmol) of 4-bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide and 53.9 mg (0.33 mmol) of (4-cyano-2-methylphenyl)boronic acid in 2.5 ml DMF, and the mixture was degassed with argon for 5 min. 16.3 mg (0.022 mmol) of 1,1′-bis(diphenylphosphino)ferrocenepalladium(II)chloride were added and the mixture was stirred at 120° C. in a preheated oil bath for 2 h. The mixture was filtered through kieselguhr and washed through with ethyl acetate. The filtrate was diluted with ethyl acetate and water and acidified with 10% strength citric acid solution, and the phases were separated. The organic phase was dried over magnesium sulphate and concentrated under reduced pressure. The residue was stirred with ethyl acetate, and the solid was filtered off and dried under high vacuum. This gave 45 mg (20% of theory, 67% pure) of the title compound.

LC-MS (Method 1): Rₜ=1.11 min; MS (ESIpos): m/z=663 [M+H]⁺.

### Example 23A

### tert-Butyl [(trans-4-{[(2S)-3-(4′-amino-2′-methylbiphenyl-4-yl)-1-oxo-1-{[4-(2H-tetrazol-5-yl)phenyl]amino}propan-2-yl]carbamoyl}cyclohexyl)methyl]carbamate

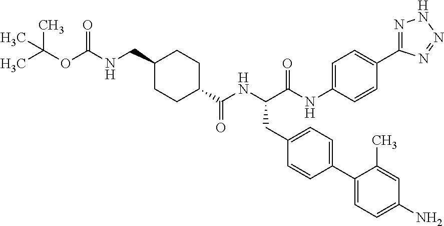 [SMILES: Cc1cc(N)ccc1-c1ccc(C[C@H](NC(=O)[C@H]2CC[C@H](CNC(=O)OC(C)(C)C)CC2)C(=O)Nc2ccc(-c3nn[nH]n3)cc2)cc1]

3.6 ml (7 2 mmol) of a 2M sodium carbonate solution in water was added to a solution of 1500 mg (2.4 mmol) of 4-bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide and 837 mg (3 6 mmol) of 3-methyl-4-(4,4,5,5-tetramethyl-1,3,2-dioxaborolan-2-yl)aniline in 25 ml DMF, and the mixture was degassed with argon for 5 min 175 mg (0.24 mmol) of 1,1′-bis(diphenylphosphino)ferrocenepalladium(II)chloride were added and the mixture was stirred at 120° C. in a preheated oil bath overnight. The mixture was filtered through kieselguhr and washed through with ethyl acetate. The filtrate was diluted with ethyl acetate and water and extracted. The aqueous phase was acidified with 10% strength citric acid solution and once more washed with ethyl acetate. The combined organic phases were dried over magnesium sulphate and concentrated under reduced pressure. The residue was stirred with dichloromethane, and the solid was filtered off and dried under high vacuum. This gave 450 mg (27% of theory) of the title compound.

LC-MS (Method 1): Rₜ=0.93 min; MS (ESIpos): m/z=653 [M+H]⁺.

### Example 24A

### N-(tent-Butoxycarbonyl)glycylglycyl-N-{4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2-methylbiphenyl-4-yl}glycinamide

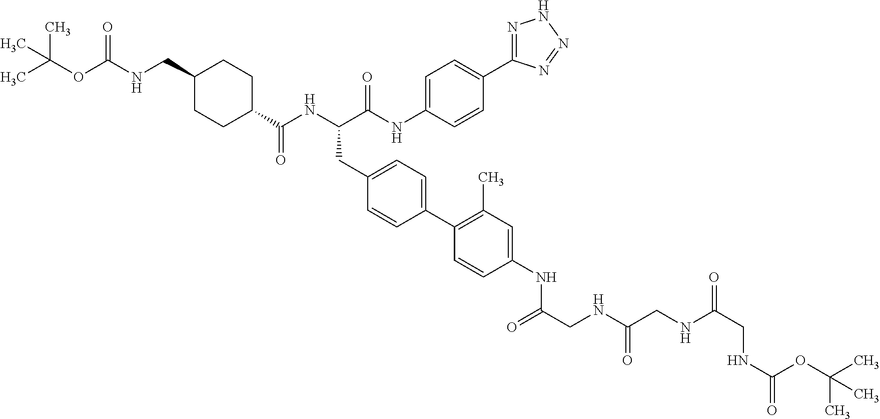 [SMILES: Cc1cc(NC(=O)CNC(=O)CNC(=O)CNC(=O)OC(C)(C)C)ccc1-c1ccc(C[C@H](NC(=O)[C@H]2CC[C@H](CNC(=O)OC(C)(C)C)CC2)C(=O)Nc2ccc(-c3nn[nH]n3)cc2)cc1]

63.6 μl (0.37 mmol) of N,N-diisopropylethylamine and 66.6 mg (0.18 mmol) of O-(7-azabenzotriazol-1-yl)-N,N,N′,N′-tetramethyluronium hexafluorophosphate were added to a solution of 100 mg (0.15 mmol) of tert-butyl [(trans-4-{[(2S)-3-(4′-amino-2′-methylbiphenyl-4-yl)-1-oxo-1-{[4-(2H-tetrazol-5-yl)phenyl]amino}propan-2-yl]carbamoyl}cyclohexyl)methyl]carbamate and 50.7 mg (0.18 mmol) of N-(tert-butoxycarbonyl)glycylglycylglycine in 2 ml of ethyl acetate. 0.5 ml of DMF was added to improve solubility, and the mixture was stirred at RT for 16 h. Water and ethyl acetate were added to the contents of the flask. The aqueous phase was once more extracted with ethyl acetate. In the organic phase, a precipitate formed, which was filtered off and washed with ethyl acetate. The solid was dried under high vacuum. This gave 60 mg (41% of theory, 92% pure) of the title compound.

LC-MS (Method 1): Rₜ=0.96 min; MS (ESIpos): m/z=924 [M+H]⁺.

### Example 25A

### tert-Butyl 4-[({4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2-methylbiphenyl-4-yl}sulphonyl)amino]piperidine-1-carboxylate

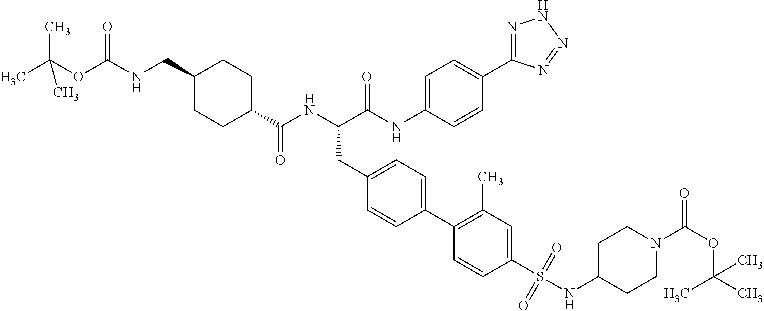 [SMILES: Cc1cc(S(=O)(=O)NC2CCN(C(=O)OC(C)(C)C)CC2)ccc1-c1ccc(C[C@H](NC(=O)[C@H]2CC[C@H](CNC(=O)OC(C)(C)C)CC2)C(=O)Nc2ccc(-c3nn[nH]n3)cc2)cc1]

Under argon, 91.2 mg (0.36 mmol) of bis(pinacolato)diborane, 145.3 mg (0.33 mmol) of tert-butyl 4-{[(4-bromo-3-methylphenyl)sulphonyl]amino}piperidine-1-carboxylate and 70.5 mg (0.72 mmol) of potassium acetate were initially charged in 2 ml of toluene, 9.8 mg (0.01 mmol) of [1,1-bis(diphenylphosphino)ferrocene]dichloropalladium/dichloromethane complex were added and the mixture was stirred at 110° C. for 3 h. The mixture was concentrated and dried under high vacuum. The residue was taken up in 2 ml of 1,2-dimethoxyethane and 0.8 ml of ethanol, and 150 mg (0.23 mmol) of 4-bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide, 9.8 mg (0.01 mmol) of 1,1-bis-(diphenylphosphino)ferrocene]dichloropalladium/dichloromethane complex and 0.24 ml (0.48 mmol) of 2M sodium carbonate solution in water were added. The mixture was stirred at 110° C. for 16 h. The mixture was separated by preparative HPLC (mobile phase: gradient of acetonitrile/water with 0.1% trifluoroacetic acid). The product-containing fractions were combined and concentrated on a rotary evaporator. The residue was dried under high vacuum. This gave 108 mg (50% of theory) of the title compound.

LC-MS (Method 1): Rₜ=1.16 min; MS (ESIpos): m/z=900 [M+H]⁺.

### Example 26A

### tert-Butyl 4-[({4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2-(trifluoromethyl)biphenyl-4-yl}sulphonyl)amino]piperidine-1-carboxylate

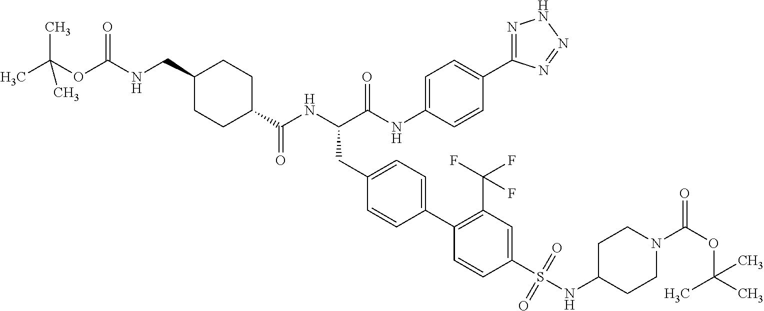 [SMILES: CC(C)(C)OC(=O)NC[C@H]1CC[C@H](C(=O)N[C@@H](Cc2ccc(-c3ccc(S(=O)(=O)NC4CCN(C(=O)OC(C)(C)C)CC4)cc3C(F)(F)F)cc2)C(=O)Nc2ccc(-c3nn[nH]n3)cc2)CC1]

Under argon, 91.2 mg (0.36 mmol) of bis(pinacolato)diborane, 163.4 mg (0.33 mmol) of tert-butyl 4-({[4-bromo-3-(trifluoromethyl)phenyl]sulphonyl}amino)piperidine-1-carboxylate and 70.5 mg (0.72 mmol) of potassium acetate were initially charged in 2 ml of toluene, 9.8 mg (0.01 mmol) of [1,1-bis(diphenylphosphino)ferrocene]dichloropalladium/dichloromethane complex were added and the mixture was stirred at 110° C. for 3 h. The mixture was concentrated and dried under high vacuum. The residue was taken up in 2 ml of 1,2-dimethoxyethane and 0.8 ml of ethanol, and 150 mg (0.24 mmol) of 4-bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide, 9.8 mg (0.01 mmol) of 1,1-bis-(diphenylphosphino)ferrocene]dichloropalladium/dichloromethane complex and 0.24 ml (0.48 mmol) of 2M sodium carbonate solution in water were added. The mixture was stirred at 110° C. for 16 h. The mixture was separated by preparative HPLC (mobile phase: gradient of acetonitrile/water with 0.1% trifluoroacetic acid). The product-containing fractions were combined and concentrated on a rotary evaporator. The residue was dried under high vacuum. This gave 32 mg (14% of theory) of the title compound.

LC-MS (Method 1): Rₜ=1.19 min; MS (ESIpos): m/z=954 [M+H]⁺.

### Example 27A

### tert-Butyl 4-[({4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2-methylbiphenyl-4-yl}sulphonyl)amino]-2-methylpiperidine-1-carboxylate

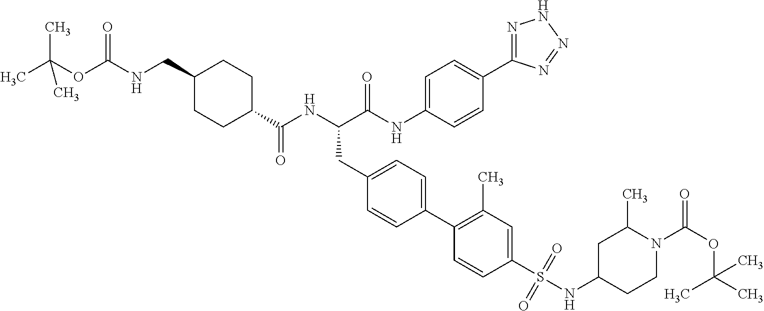 [SMILES: Cc1cc(S(=O)(=O)NC2CCN(C(=O)OC(C)(C)C)C(C)C2)ccc1-c1ccc(C[C@H](NC(=O)[C@H]2CC[C@H](CNC(=O)OC(C)(C)C)CC2)C(=O)Nc2ccc(-c3nn[nH]n3)cc2)cc1]

Under argon, 91.2 mg (0.36 mmol) of bis(pinacolato)diborane, 150 mg (0.33 mmol) of tert-butyl 4-{[(4-bromo-3-methylphenyl)sulphonyl]amino}-2-methylpiperidine-1-carboxylate and 70.5 mg (0.72 mmol) of potassium acetate were initially charged in 2 ml of toluene, 9.8 mg (0.01 mmol) of [1,1-bis(diphenylphosphino)ferrocene]dichloropalladium/dichloromethane complex were added and the mixture was stirred at 110° C. for 3 h. The mixture was concentrated and dried under high vacuum. The residue was taken up in 2 ml of 1,2-dimethoxyethane and 0.8 ml of ethanol, and 150 mg (0.24 mmol) of 4-bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide, 9.8 mg (0.01 mmol) of 1,1-bis-(diphenylphosphino)ferrocene]dichloropalladium/dichloromethane complex and 0.24 ml (0.48 mmol) of 2M sodium carbonate solution in water were added. The mixture was stirred at 110° C. for 16 h. The mixture was separated by preparative HPLC (mobile phase: gradient of acetonitrile/water with 0.1% trifluoroacetic acid). The product-containing fractions were combined and concentrated on a rotary evaporator. The residue was dried under high vacuum. This gave 104 mg (47% of theory) of the title compound.

LC-MS (Method 1): Rₜ=1.19 min; MS (ESIpos): m/z=914 [M+H]⁺.

### Example 28A

### tert-Butyl (3R)-3-[({4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2-methylbiphenyl-4-yl}sulphonyl)amino]pyrrolidine-1-carboxylate

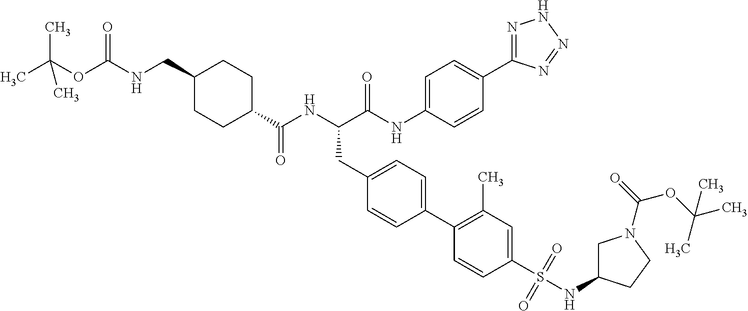 [SMILES: Cc1cc(S(=O)(=O)N[C@@H]2CCN(C(=O)OC(C)(C)C)C2)ccc1-c1ccc(C[C@H](NC(=O)[C@H]2CC[C@H](CNC(=O)OC(C)(C)C)CC2)C(=O)Nc2ccc(-c3nn[nH]n3)cc2)cc1]

Under argon, 91.2 mg (0.36 mmol) of bis(pinacolato)diborane, 140.5 mg (0.33 mmol) of tert-butyl (3 R)-3-{[(4-bromo-3-methylphenyl)sulphonyl]amino}pyrrolidine-1-carboxylate and 70.5 mg (0.72 mmol) of potassium acetate were initially charged in 2 ml of toluene, 9.8 mg (0.01 mmol) of [1,1-bis(diphenylphosphino)ferrocene]dichloropalladium/dichloromethane complex were added and the mixture was stirred at 110° C. for 3 h. The mixture was concentrated and dried under high vacuum. The residue was taken up in 2 ml of 1,2-dimethoxyethane and 0.8 ml of ethanol, and 150 mg (0.23 mmol) of 4-bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide, 9.8 mg (0.01 mmol) of 1,1-bis-(diphenylphosphino)ferrocene]dichloropalladium/dichloromethane complex and 0.24 ml (0.48 mmol) of 2M sodium carbonate solution in water were added. The mixture was stirred at 110° C. for 16 h. The mixture was separated by preparative HPLC (mobile phase: gradient of acetonitrile/water with 0.1% trifluoroacetic acid). The product-containing fractions were combined and concentrated on a rotary evaporator. The residue was dried under high vacuum. This gave 120 mg (56% of theory) of the title compound.

LC-MS (Method 1): Rₜ=1.13 min; MS (ESIpos): m/z=886 [M+H]⁺.

### Example 29A

### tert-Butyl [(trans-4-{[(2S)-3-[4′-(methylsulphonyl)-2′-(trifluoromethyl)biphenyl-4-yl]-1-oxo-1-{[4-(2H-tetrazol-5-yl)phenyl]amino}propan-2-yl]carbamoyl}cyclohexyl)methyl]carbamate

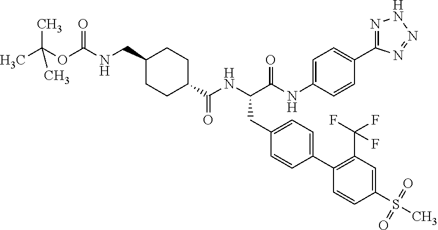 [SMILES: CC(C)(C)OC(=O)NC[C@H]1CC[C@H](C(=O)N[C@@H](Cc2ccc(-c3ccc(S(C)(=O)=O)cc3C(F)(F)F)cc2)C(=O)Nc2ccc(-c3nn[nH]n3)cc2)CC1]

100 mg (0.16 mmol) of 4-bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide, 64.2 mg (0.24 mmol) of [4-(methylsulphonyl)-2-(trifluoromethyl)phenyl]boronic acid and 18.44 mg (0.016 mmol) of tetrakis(triphenylphosphine)palladium(0) were initially charged in 1.5 ml of 1,2-dimethoxyethane and 0.5 ml of ethanol After the addition of 0.5 ml of 2M sodium carbonate solution in water, the mixture was stirred at 100° C. for 24 h. Another 20 mg of tetrakis(triphenylphosphine)palladium(0) were added, and the mixture was stirred at 110° C. for 20 h. The mixture was separated by preparative HPLC (mobile phase: gradient of acetonitrile/water with 0.1% trifluoroacetic acid). The product-containing fractions were combined and concentrated on a rotary evaporator. The residue was dried under high vacuum. 22 mg (14% of theory, 80% purity) of the title compound were obtained.

LC-MS (Method 1): Rₜ=1.04 min; MS (ESIpos): m/z=770 [M+H]⁺.

### Example 30A

### tert-Butyl {[trans-4-({(2S)-3-[4′-(cyclopropylsulphamoyl)-2′-methylbiphenyl-4-yl]-1-oxo-1-[(2-oxo-2,3-dihydro-1H-benzimidazol-5-yl)amino]propan-2-yl}carbamoyl)cyclohexyl]methyl}carbamate

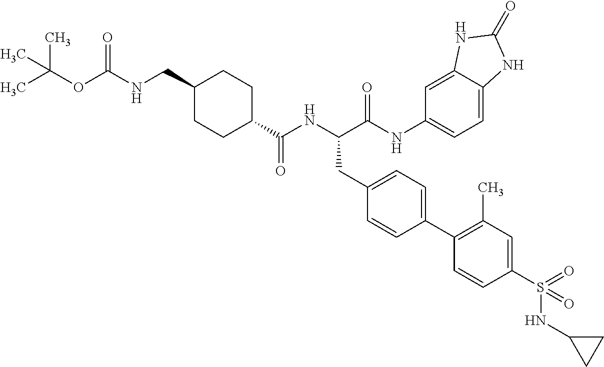 [SMILES: Cc1cc(S(=O)(=O)NC2CC2)ccc1-c1ccc(C[C@H](NC(=O)[C@H]2CC[C@H](CNC(=O)OC(C)(C)C)CC2)C(=O)Nc2ccc3[nH]c(=O)[nH]c3c2)cc1]

28.2 mg (24.4 μmol) of tetrakis(triphenylphosphine)palladium(0), 77.6 mg (0 7 mmol) of sodium carbonate and 0.37 ml (20.4 mmol) of water were added to a solution of 150 mg (0.24 mmol) of 4-bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide and 98.3 mg (0.37 mmol) of [4-(cyclopropylsulphamoyl)-2-methylphenyl]boronic acid in 2 ml of DMSO. The mixture was treated in the microwave at 110° C. for 90 min. The mixture was filtered and purified by preparative HPLC (Method 11). This gave 60 mg (33% of theory) of the title compound.

LC-MS (Method 5): Rₜ=1.14 min; MS (ESIpos): m/z=745.4 [M+H]⁺.

### Example 31A

### tert-Butyl {[trans-4-({(2S)-3-[4′-(dimethylsulphamoyl)-2′-methylbiphenyl-4-yl]-1-oxo-1-[(2-oxo-2,3-dihydro-1H-benzimidazol-5-yl)amino]propan-2-yl}carbamoyl)cyclohexyl]methyl}carbamate

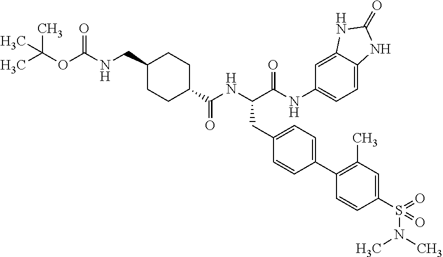 [SMILES: Cc1cc(S(=O)(=O)N(C)C)ccc1-c1ccc(C[C@H](NC(=O)[C@H]2CC[C@H](CNC(=O)OC(C)(C)C)CC2)C(=O)Nc2ccc3[nH]c(=O)[nH]c3c2)cc1]

28.2 mg (24.4 μmol) of tetrakis(triphenylphosphine)palladium(0), 77.6 mg (0 7 mmol) of sodium carbonate and 0.37 ml (20.4 mmol) of water were added to a solution of 150 mg (0.24 mmol) of 4-bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide and 93.7 mg (0.37 mmol) of [4-(dimethylsulphamoyl)-2-methylphenyl]boronic acid in 2 ml of DMSO. The mixture was treated in the microwave at 110° C. for 90 min. The mixture was filtered and purified by preparative HPLC (Method 11). This gave 68 mg (38% of theory) of the title compound.

LC-MS (Method 5): Rₜ=1.17 min; MS (ESIpos): m/z=733.4 [M+H]⁺.

### Example 32A

### tert-Butyl {[trans-4-({(2S)-3-[4′-(isopropylsulphamoyl)-2′-methylbiphenyl-4-yl]-1-oxo-1-[(2-oxo-2,3-dihydro-1H-benzimidazol-5-yl)amino]propan-2-yl}carbamoyl)cyclohexyl]methyl}carbamate

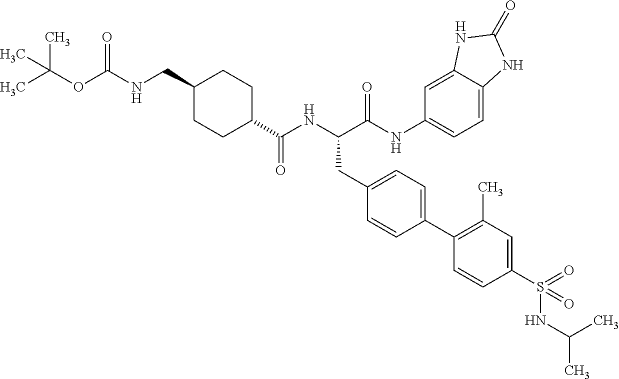 [SMILES: Cc1cc(S(=O)(=O)NC(C)C)ccc1-c1ccc(C[C@H](NC(=O)[C@H]2CC[C@H](CNC(=O)OC(C)(C)C)CC2)C(=O)Nc2ccc3[nH]c(=O)[nH]c3c2)cc1]

28.2 mg (24.4 μmol) of tetrakis(triphenylphosphine)palladium(0), 77.6 mg (0 7 mmol) of sodium carbonate and 0.37 ml (20.4 mmol) of water were added to a solution of 150 mg (0.24 mmol) of 4-bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide and 99.1 mg (0.37 mmol) of [4-(isopropylsulphamoyl)-2-methylphenyl]boronic acid in 2 ml of DMSO. The mixture was treated in the microwave at 110° C. for 90 min. The mixture was filtered and purified by preparative HPLC (Method 11). This gave 54 mg (29% of theory) of the title compound.

LC-MS (Method 5): Rₜ=1.17 min; MS (ESIpos): m/z=747.4 [M+H]⁺.

### Example 33A

### tert-Butyl {[trans-4-({(2S)-3-[2′-methyl-4′-(pyrrolidin-1-ylsulphonyl)biphenyl-4-yl]-1-oxo-1-[(2-oxo-2,3-dihydro-1H-benzimidazol-5-yl)amino]propan-2-yl}carbamoyl)cyclohexyl]methyl}carbamate

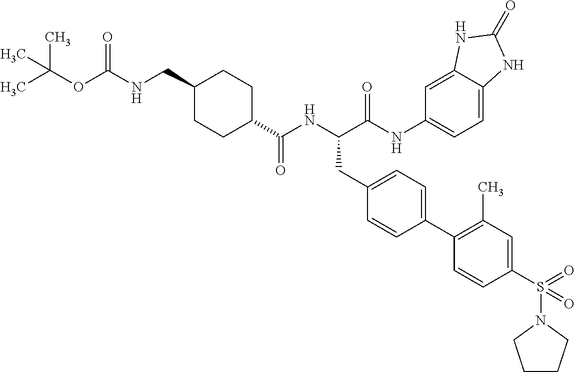 [SMILES: Cc1cc(S(=O)(=O)N2CCCC2)ccc1-c1ccc(C[C@H](NC(=O)[C@H]2CC[C@H](CNC(=O)OC(C)(C)C)CC2)C(=O)Nc2ccc3[nH]c(=O)[nH]c3c2)cc1]

28.2 mg (24.4 μmol) of tetrakis(triphenylphosphine)palladium(0), 77.6 mg (0 7 mmol) of sodium carbonate and 0.37 ml (20.4 mmol) of water were added to a solution of 150 mg (0.24 mmol) of 4-bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide and 103.7 mg (0.37 mmol) of [2-methyl-4-(pyrrolidin-1-ylsulphonyl)phenyl]boronic acid in 2 ml of DMSO. The mixture was treated in the microwave at 110° C. for 90 min. The mixture was filtered and purified by preparative HPLC (Method 11). This gave 63 mg (34% of theory) of the title compound.

LC-MS (Method 5): Rₜ=1.21 min; MS (ESIpos): m/z=759.4 [M+H]⁺.

### Example 34A

### tert-Butyl {[trans-4-({(2S)-3-[4′-(diethylsulphamoyl)-2′-methylbiphenyl-4-yl]-1-oxo-1-[(2-oxo-2,3-dihydro-1H-benzimidazol-5-yl)amino]propan-2-yl}carbamoyl)cyclohexyl]methyl}carbamate

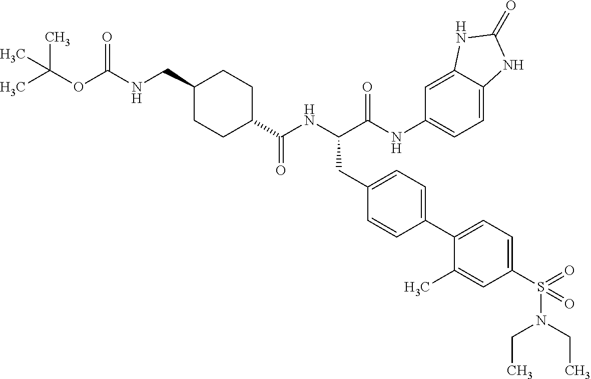 [SMILES: CCN(CC)S(=O)(=O)c1ccc(-c2ccc(C[C@H](NC(=O)[C@H]3CC[C@H](CNC(=O)OC(C)(C)C)CC3)C(=O)Nc3ccc4[nH]c(=O)[nH]c4c3)cc2)c(C)c1]

28.2 mg (24.4 μmol) of tetrakis(triphenylphosphine)palladium(0), 77.6 mg (0 7 mmol) of sodium carbonate and 0.37 ml (20.4 mmol) of water were added to a solution of 150 mg (0.24 mmol) of 4-bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide and 104.5 mg (0.37 mmol) of [4-(diethylsulphamoyl)-2-methylphenyl]boronic acid in 2 ml of DMSO. The mixture was treated in the microwave at 110° C. for 90 min. The mixture was filtered and purified by preparative HPLC (Method 11). This gave 58 mg (31% of theory) of the title compound.

LC-MS (Method 5): Rₜ=1.25 min; MS (ESIpos): m/z=761.4 [M+H]⁺.

### Example 35A

### tert-Butyl {[trans-4-({(2S)-3-[2′-methyl-4′-(methylsulphamoyl)biphenyl-4-yl]-1-oxo-1-[(2-oxo-2,3-dihydro-1H-benzimidazol-5-yl)amino]propan-2-yl}carbamoyl)cyclohexyl]methyl}carbamate

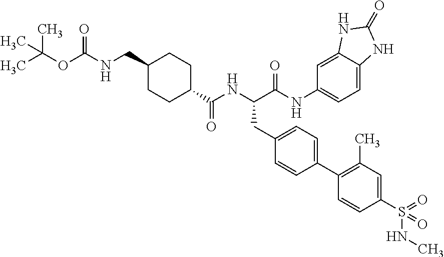 [SMILES: CNS(=O)(=O)c1ccc(-c2ccc(C[C@H](NC(=O)[C@H]3CC[C@H](CNC(=O)OC(C)(C)C)CC3)C(=O)Nc3ccc4[nH]c(=O)[nH]c4c3)cc2)c(C)c1]

28.2 mg (24.4 μmol) of tetrakis(triphenylphosphine)palladium(0), 77.6 mg (0 7 mmol) of sodium carbonate and 0.37 ml (20.4 mmol) of water were added to a solution of 150 mg (0.24 mmol) of 4-bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide and 104.5 mg (0.37 mmol) of [2-methyl-4-(methylsulphamoyl)phenyl]boronic acid in 2 ml of DMSO. The mixture was treated in the microwave at 110° C. for 90 min. The mixture was filtered and purified by preparative HPLC (Method 9). This gave 24 mg (14% of theory) of the title compound.

LC-MS (Method 4): Rₜ=1.06 min; MS (ESIpos): m/z=719.4 [M+H]⁺.

### Example 36A

### tert-Butyl [(trans-4-{[(2S)-1-{[4-(3-chloro-4H-1,2,4-triazol-5-yl)phenyl]amino}-3-{4′-[(2-hydroxyethyl)sulphamoyl]biphenyl- 4-yl}-1-oxopropan-2-yl]carbamoyl}cyclohexyl)methyl]carbamate

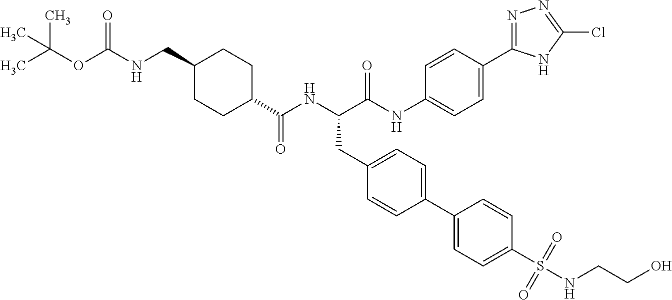 [SMILES: CC(C)(C)OC(=O)NC[C@H]1CC[C@H](C(=O)N[C@@H](Cc2ccc(-c3ccc(S(=O)(=O)NCCO)cc3)cc2)C(=O)Nc2ccc(-c3nnc(Cl)[nH]3)cc2)CC1]

26.3 mg (22.7 μmol) of tetrakis(triphenylphosphine)palladium(0), 72.3 mg (0.68 mmol) of sodium carbonate and 0.34 ml (19.0 mmol) of water were added to a solution of 150 mg (0.23 mmol) of 4-bromo-N-alpha-[(trans-4-{[(tert-butoxycarbonyl)amino]methyl}cyclohexyl)carbonyl]-N-[4-(3-chloro-4H-1,2,4-triazol-5-yl)phenyl]-L-phenylalaninamide and 85.2 mg (0.34 mmol) of {4-[(2-hydroxyethyl)sulphamoyl]phenyl}boronic acid in 2 ml of DMSO. The mixture was treated in the microwave at 110° C. for 90 min. The reaction was incomplete. 0.1 eq. of tetrakis(triphenylphosphine)palladium(0) were added, and the mixture was stirred in a microwave at 110° C. for 2 h. The mixture was filtered and purified by preparative HPLC (Method 10). This gave 21 mg (11% of theory) of the title compound.

LC-MS (Method 5): Rₜ=1.15 min; MS (ESIpos): m/z=780.3 [M+H]⁺.

### Example 37A

### tert-Butyl [(trans-4-{[(2S)-1-{[4-(3-chloro-4H-1,2,4-triazol-5-yl)phenyl]amino}-3-(2′-methyl-4′-sulphamoylbiphenyl-4-yl)-1-oxopropan-2-yl]carbamoyl}cyclohexyl)methyl]carbamate

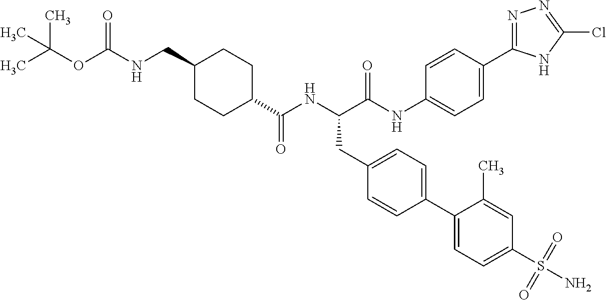 [SMILES: Cc1cc(S(N)(=O)=O)ccc1-c1ccc(C[C@H](NC(=O)[C@H]2CC[C@H](CNC(=O)OC(C)(C)C)CC2)C(=O)Nc2ccc(-c3nnc(Cl)[nH]3)cc2)cc1]

24.5 mg (21 μmol) of tetrakis(triphenylphosphine)palladium(0), 67.5 mg (0.64 mmol) of sodium carbonate and 0.32 ml (17.7 mmol) of water were added to a solution of 150 mg (0.21 mmol) of N-alpha-[(trans-4-{[(tert-butoxycarbonyl)amino]methyl}cyclohexyl)carbonyl]-N-[4-(3-chloro-4H-1,2,4-triazol-5-yl)phenyl]-4-iodo-L-phenylalaninamide and 70.6 mg (0.32 mmol) of {(2-methyl-4-sulphamoylphenyl)boronic acid in 2 ml of DMSO. The mixture was treated in the microwave at 110° C. for 90 min. The reaction was incomplete. 0.1 eq. of tetrakis(triphenylphosphine)palladium(0) were added, and the mixture was stirred in a microwave at 110° C. for 1 h. The mixture was filtered and purified by preparative HPLC (Method 11). This gave 17 mg (11% of theory) of the title compound.

LC-MS (Method 5): Rₜ=0.79 min; MS (ESIpos): m/z=750.4 [M+H]⁺.

### Example 38A

### tert-Butyl [(trans-4-{[(2S)-1-{[4-(3-chloro-4H-1,2,4-triazol-5-yl)phenyl]amino}-1-oxo-3-(4′-sulphamoylbiphenyl-4-yl)propan-2-yl]carbamoyl}cyclohexyl)methyl]carbamate

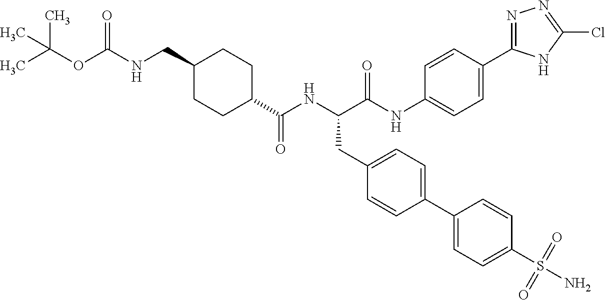 [SMILES: CC(C)(C)OC(=O)NC[C@H]1CC[C@H](C(=O)N[C@@H](Cc2ccc(-c3ccc(S(N)(=O)=O)cc3)cc2)C(=O)Nc2ccc(-c3nnc(Cl)[nH]3)cc2)CC1]

24.5 mg (21 μmol) of tetrakis(triphenylphosphine)palladium(0), 67.5 mg (0.64 mmol) of sodium carbonate and 0.32 ml (17.7 mmol) of water were added to a solution of 150 mg (0.21 mmol) of N-alpha-[(trans-4-{[(tert-butoxycarbonyl)amino]methyl}cyclohexyl)carbonyl]-N-[4-(3-chloro-4H-1,2,4-triazol-5-yl)phenyl]-4-iodo-L-phenylalaninamide and 90.1 mg (0.32 mmol) of 4-(4,4,5,5-tetramethyl-1,3,2-dioxaborolan-2-yl)benzenesulphonamide in 2 ml of DMSO. The mixture was treated in the microwave at 110° C. for 90 min. The reaction was incomplete. 0.1 eq. of tetrakis(triphenylphosphine)palladium(0) were added, and the mixture was stirred in a microwave at 110° C. for 1 h. The mixture was filtered and purified by preparative HPLC (Method 11). This gave 19 mg (12% of theory) of the title compound.

LC-MS (Method 5): Rₜ=0.73 min; MS (ESIpos): m/z=736.4 [M+H]⁺.

### Example 39A

### tert-Butyl [(trans-4-{[(2S)-3-[4′-(dimethylsulphamoyl)-2′-methylbiphenyl-4-yl]-1-{[3-fluoro-4-(2H-tetrazol-5-yl)phenyl]amino}-1-oxopropan-2-yl]carbamoyl}cyclohexyl)methyl]carbamate

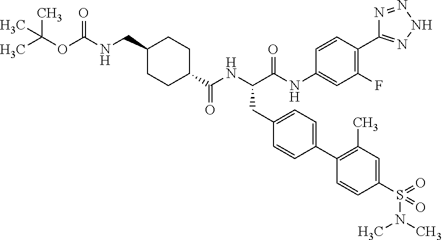 [SMILES: Cc1cc(S(=O)(=O)N(C)C)ccc1-c1ccc(C[C@H](NC(=O)[C@H]2CC[C@H](CNC(=O)OC(C)(C)C)CC2)C(=O)Nc2ccc(-c3nn[nH]n3)c(F)c2)cc1]

26.9 mg (23.3 μmol) of tetrakis(triphenylphosphine)palladium(0), 74.0 mg (0.7 mmol) of sodium carbonate and 0.35 ml (19.4 mmol) of water were added to a solution of 150 mg (0.23 mmol) of 4-bromo-N-alpha-[(trans-4-{[(tert-butoxycarbonyl)amino]methyl}cyclohexyl)carbonyl]-N-[3-fluoro-4-(2H-tetrazol-5-yl)phenyl]-L-phenylalaninamide and 89.3 mg (0.35 mmol) of [4-(dimethylsulphamoyl)-2-methylphenyl]boronic acid in 2 ml of DMSO. The mixture was treated in the microwave at 110° C. for 90 min. The mixture was filtered and purified by preparative HPLC (Method 10). This gave 72 mg (41% of theory) of the title compound.

LC-MS (Method 4): Rₜ=1.27 min; MS (ESIpos): m/z=763.5 [M+H]⁺.

### Example 40A

### tert-Butyl [(trans-4-{[(2S)-3-[4′-methoxy-2′-(trifluoromethyl)biphenyl-4-yl]-1-oxo-1-{[4-(2H-tetrazol-5-yl)phenyl]amino}propan-2-yl]carbamoyl}cyclohexyl)methyl]carbamate

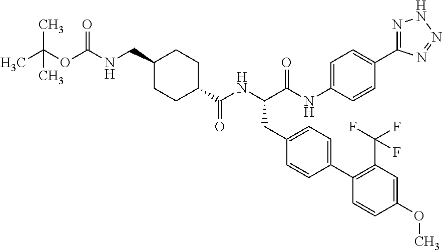 [SMILES: COc1ccc(-c2ccc(C[C@H](NC(=O)[C@H]3CC[C@H](CNC(=O)OC(C)(C)C)CC3)C(=O)Nc3ccc(-c4nn[nH]n4)cc3)cc2)c(C(F)(F)F)c1]

Under argon, 18.4 mg (0.016 mmol) of tetrakis(triphenylphosphine)palladium(0) were added to a solution of 100 mg (0.16 mmol) of 4-bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide in 1.4 ml of 1,2-dimethoxyethane, and the mixture was stirred for 10 min. 105.3 mg (0.48 mmol) of [4-methoxy-2-(trifluoromethyl)phenyl]boronic acid in 0.5 ml of ethanol were added dropwise, and the mixture was stirred at RT for 10 min 1 2 ml of 2M sodium carbonate solution in water were then added to the mixture. The mixture was stirred at reflux for 3 h. The mixture was diluted with a little methanol, filtered and purified by preparative HPLC (mobile phase: gradient of acetonitrile/water with 0.1% trifluoroacetic acid). The product-containing fractions were combined and concentrated on a rotary evaporator. This gave 83 mg (62% of theory) of the title compound.

LC-MS (Method 1): Rₜ=1.21 min; MS (ESIpos): m/z=722 [M+H]⁺.

### Example 41A

### 4-Bromo-N-alpha-[(trans-4-{[(tert-butoxycarbonyl)amino]methyl}cyclohexyl)carbonyl]-N-[4-(5-oxo-4,5-dihydro-1,2,4-oxadiazol-3-yl)phenyl]-L-phenylalaninamide

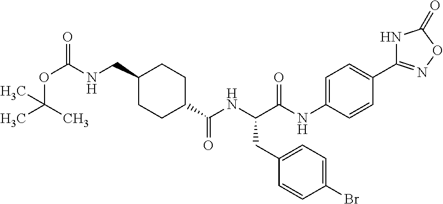

N,N-Diisopropylethylamine (0.9 ml, 5 mmol) was added to a solution of 4-bromo-N-[(trans-4-{[(tert-butoxycarbonyl)amino]methyl}cyclohexyl)carbonyl]-L-phenylalanine (1000 mg, 2 mmol) and 3-(4-aminophenyl)-4,5-dihydro-1,2,4-oxadiazol-5-one (403 mg, 2 mmol) in dimethylformamide (15 ml). A 2,4,6-tripropyl-1,3,5,2,4,6-trioxatriphosphinane 2,4,6-trioxide solution (50% in dimethylformamide, 1580 mg, 5 mmol) and, until dissolution, dimethylformamide were added to the suspension, and the mixture was then stirred at RT for 16 h. The reaction mixture was stirred into ethyl acetate (1200 ml), and washed with water (150 ml) and once with saturated aqueous sodium chloride solution. The organic phase was dried with sodium sulphate and the solvent was removed. The crude product was stirred with acetonitrile and filtered off with suction. This gave 540 mg (38% of theory, 94% pure) of the title compound.

¹H-NMR (400 MHz, DMSO-d₆): δ=ppm 0.68-0.98 (m, 2H), 1.05-1.31 (m, 4H), 1.39 (s, 9H), 1.46-1.76 (m, 4H), 1.98-2.15 (m, 1H), 2.65-3.07 (m, 4H), 4.56-4.71 (m, 1H), 6.71-6.83 (m, 1H), 7.25 (d, 2H), 7.47 (d, 2H), 7.72-7.84 (m, 4H), 8.10-8.20 (m, 1H), 10.45 (s, 1H), 12.86 (br. s, 1H).

LC-MS (Method 1): Rₜ=1.12 min; MS (ESIneg): m/z=640 [M−H]⁻.

### Example 42A

### 4-Bromo-N-[4-(dimethylamino)cyclohexyl]-3-(trifluoromethyl)benzenesulphonamide

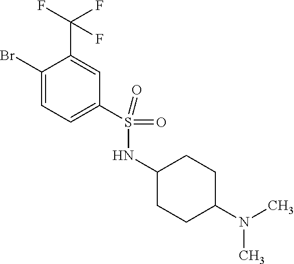 [SMILES: CN(C)C1CCC(NS(=O)(=O)c2ccc(Br)c(C(F)(F)F)c2)CC1]

4-Bromo-3-(trifluoromethyl)benzenesulphonyl chloride (500 mg, 1.55 mmol) was dissolved in dichloromethane (10 ml) and cooled to 0° C., and N,N-dimethylcyclohexane-1,4-diamine (286 mg, 2.0 mmol) and N,N-diisopropylethylamine (0.67 ml, 3.9 mmol) were added. The reaction mixture was stirred at RT overnight, taken up in dichloromethane and washed twice with saturated aqueous ammonium chloride solution. The combined aqueous phases were re-extracted once with dichloromethane. The combined organic phases were washed with a saturated aqueous sodium chloride solution, dried over sodium sulphate, filtered and concentrated. The residue was purified chromatographically by HPLC (Waters Autopurificationsystem: Pump 254, Sample Manager 2767, CFO, DAD 2996, ELSD 2424, SQD 3100; XBrigde C18 5 μm 100 mm×30 mm; mobile phase A: water+0.2% ammonia (32% strenght), mobile phase B: acetonitrile; gradient: 0-8 min 45-70% B; flow rate: 70 ml/min) This gave 164 mg (25% of theory) of the title compound.

¹H NMR (400 MHz, DMSO-d₆): δ=ppm 1.12 (m, 4H), 1.58-1.72 (m, 4H), 1.92-2.01 (m, 1H), 2.07 (s, 6H), 2.86-3.00 (m, 1H), 7.97 (m, 2H), 8.09-8.17 (m, 2H).

LC-MS (Method 4): Rₜ=0.89 min; MS (ESIpos): m/z=431.1 [M+H]⁺.

### Example 43A

### 4-Bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide

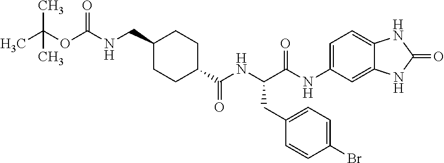 [SMILES: CC(C)(C)OC(=O)NC[C@H]1CC[C@H](C(=O)N[C@@H](Cc2ccc(Br)cc2)C(=O)Nc2ccc3[nH]c(=O)[nH]c3c2)CC1]

N,N-Diisopropylethylamine (4.5 ml, 26 mmol) was added to a solution of 4-bromo-N-[(trans-4-{[(tert-butoxycarbonyl)amino]methyl}cyclohexyl)carbonyl]-L-phenylalanine (5000 mg, 10 mmol) and 5-amino-1,3-dihydro-2H-benzimidazol-2-one (1851 mg, 12 mmol) in ethyl acetate (70 ml). The suspension was admixed with a 2,4,6-tripropyl-1,3,5,2,4,6-trioxatriphosphinane 2,4,6-trioxide solution (50% in dimethylformamide, 7898 mg, 12 mmol) and with dimethylformamide (20 ml) until dissolution, and then the mixture was stirred at RT for 16 h. The reaction mixture was stirred into ethyl acetate (600 ml), and washed three times with water (300 ml) and once with saturated aqueous sodium chloride solution (250 ml). The precipitate in the organic phase was filtered off and washed with ethyl acetate. The solvent of the filtrate was removed and the residue was dried under high vacuum. This gave 4021 mg (62% of theory) of the title compound.

¹H-NMR (400 MHz, DMSO-d₆): δ=ppm 0.68-0.89 (m, 2H), 1.17 (m, 3H), 1.37 (s, 9H), 1.66 (m, 4H), 2.02-2.15 (m, 1H), 2.74 (m, 3H), 2.93-3.07 (m, 1H), 3.98-4.09 (dd, 1H), 4.52-4.66 (dd, 1H), 6.72-6.88 (m, 2H), 7.02 (dd, 1H), 7.25 (d, 2H), 7.38-7.53 (m, 3H), 8.10 (d, 1H), 10.04 (s, 1H), 10.51 (s, 1H), 10.59 (s, 1H).

LC-MS (Method 1): Rₜ=1.00 min; MS (ESIneg): m/z=612 [M−H]⁻.

### Example 44A

### N-alpha-[(trans-4-{[(tert-Butoxycarbonyl)amino]methyl}cyclohexyl)carbonyl]-N-(2-oxo-2,3-dihydro-1H-benzimidazol-5-yl)-4-(4,4,5,5-tetramethyl-1,3,2-dioxaborolan-2-yl)-L-phenylalaninamide

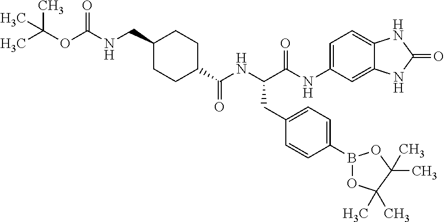

4-Bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide (5.0 g, 8.1 mmol) and bis(pinacolato)diborane (3.1 g, 12 2 mmol) were dissolved in 60 ml of dimethyl sulphoxide, freed of air with argon and blanketed. 1,1′-Bis(diphenylphosphino)ferrocenedichloropalladium(II) (332 mg, 0.41 mmol) and potassium acetate (2.4 g 24 4 mmol) were added and the reaction mixture was stirred at 110° C. for 4 h and subsequently reacted further as crude product.

LC-MS (Method 4): Rₜ=1.27 min; MS (ESIpos): m/z=662.5 [M+H]⁺.

### Example 45A

### tert-Butyl {[trans-4-({(2S)-3-[4′-{[4-(dimethylamino)cyclohexyl]sulphamoyl}-2′-(trifluoromethyl)biphenyl-4-yl]-1-oxo-1-[(2-oxo-2,3-dihydro-1H-benzimidazol-5-yl)amino]propan-2-yl}carbamoyl)cyclohexyl]methyl}carbamate

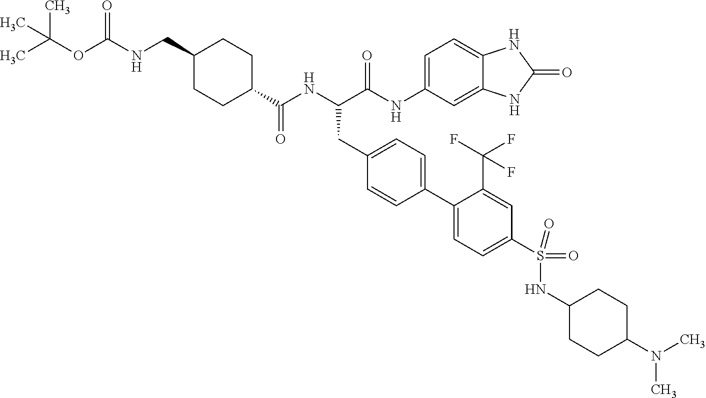

N-alpha-[(trans-4-{[(tert-Butoxycarbonyl)amino]methyl}cyclohexyl)carbonyl]-N-(2-oxo-2,3-dihydro-1H-benzimidazol-5-yl)-4-(4,4,5,5-tetramethyl-1,3,2-dioxaborolan-2-yl)-L-phenylalaninamide (150 mg, 0.23 mmol) and 4-bromo-N-[4-(dimethylamino)cyclohexyl]-3-(trifluoromethyl)benzenesulphonamide (107 mg, 0.25 mmol) were dissolved in dimethyl sulphoxide (2 ml), and tetrakis(triphenylphosphine)palladium (26 mg, 0.022 mmol), sodium carbonate (72 mg, 0.68 mmol) and water (0.34 ml) were added. The reaction mixture was stirred in a microwave (Biotage Initiator) at 110° C. for 90 min, 1,1′-bis(diphenylphosphino)ferrocene-dichloropalladium(II) (9 mg, 0.01 mmol) was added and stirring in the microwave was continued at 110° C. for 2 h. The reaction mixture was purified by chromatography via HPLC (Method 8). This gave 34 mg (17% of theory) of the title compound.

LC-MS (Method 4): Rₜ=0.99 min; MS (ESIpos): m/z=884.6 [M+H]⁺.

### Example 46A

### 4-Bromo-N-alpha-[(trans-4-{[(tert-butoxycarbonyl)amino]methyl}cyclohexyl)carbonyl]-N-(3-oxo-2,3-dihydro-1H-indazol-6-yl)-L-phenylalaninamide

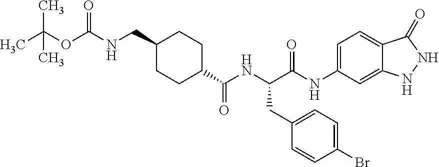

N,N-Diisopropylethylamine is added to a solution of 4-bromo-N-[(trans-4-{[(tert-butoxycarbonyl)amino]methyl}cyclohexyl)carbonyl]-L-phenylalanine and 6-amino-1,2-dihydro-3H-indazol-3-one in ethyl acetate. A 2,4,6-tripropyl-1,3,5,2,4,6-trioxatriphosphinane 2,4,6-trioxide solution (50% in dimethylformamide) is added to the reaction mixture, and the mixture is then stirred overnight at RT. The reaction mixture is worked up by methods known to those skilled in the art and the residue is separated by means of preparative HPLC. This gives the title compound.

### Example 47A

### tert-Butyl {[trans-4-({(2S)-3-[4′-(cyclopropylsulphamoyl)-2′-methylbiphenyl-4-yl]-1-oxo-1-[(3-oxo-2,3-dihydro-1H-indazol-6-yl)amino]propan-2-yl}carbamoyl)cyclohexyl]methyl}carbamate

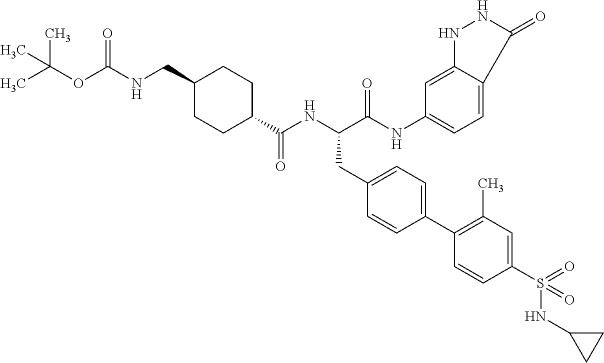 [SMILES: Cc1cc(S(=O)(=O)NC2CC2)ccc1-c1ccc(C[C@H](NC(=O)[C@H]2CC[C@H](CNC(=O)OC(C)(C)C)CC2)C(=O)Nc2ccc3c(=O)[nH][nH]c3c2)cc1]

Tetrakis(triphenylphosphine)palladium(0), sodium carbonate and water are added to a solution of 4-bromo-N-alpha-[(trans-4-{[(tert-butoxycarbonyl)amino]methyl}cyclohexyl)carbonyl]-N-(3-oxo-2,3-dihydro-1H-indazol-6-yl)-L-phenylalaninamide and [4-(cyclopropylsulphamoyl)-2-methylphenyl]boronic acid in DMSO. The mixture is treated in the microwave at 110° C. for 1 to 3 hours. The mixture is filtered and purified by preparative HPLC. This gives the title compound.

### Example 48A

### tert-Butyl {[trans-4-({(2S)-3-[4′-(dimethylsulphamoyl)-2′-methylbiphenyl-4-yl]-1-oxo-1-[(3-oxo-2,3-dihydro-1H-indazol-6-yl)amino]propan-2-yl}carbamoyl)cyclohexyl]methyl}carbamate

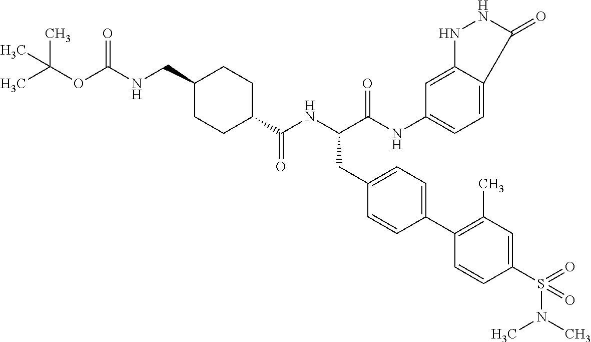 [SMILES: Cc1cc(S(=O)(=O)N(C)C)ccc1-c1ccc(C[C@H](NC(=O)[C@H]2CC[C@H](CNC(=O)OC(C)(C)C)CC2)C(=O)Nc2ccc3c(=O)[nH][nH]c3c2)cc1]

Tetrakis(triphenylphosphine)palladium(0), sodium carbonate and water are added to a solution of 4-bromo-N-alpha-[(trans-4-{[(tert-butoxycarbonyl)amino]methyl}cyclohexyl)carbonyl]-N-(3-oxo-2,3-dihydro-1H-indazol-6-yl)-L-phenylalaninamide and [4-(dimethylsulphamoyl)-2-methylphenyl]boronic acid in DMSO. The mixture is treated in the microwave at 110° C. for 1 to 3 hours. The mixture is filtered and purified by preparative HPLC. This gives the title compound.

### Example 49A

### tert-Butyl {[trans-4-({(2S)-3-[4′-(isopropylsulphamoyl)-2′-methylbiphenyl-4-yl]-1-oxo-1-[(3-oxo-2,3-dihydro-1H-indazol-6-yl)amino]propan-2-yl}carbamoyl)cyclohexyl]methyl}carbamate

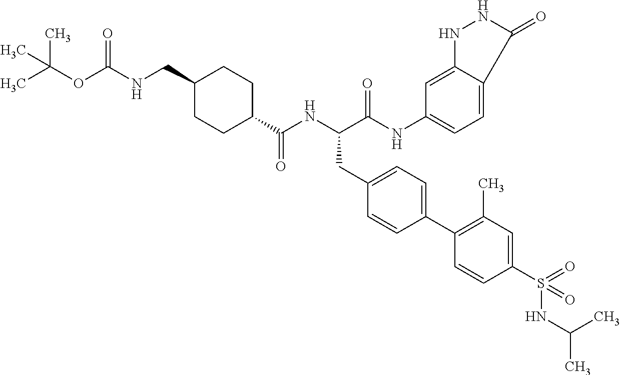 [SMILES: Cc1cc(S(=O)(=O)NC(C)C)ccc1-c1ccc(C[C@H](NC(=O)[C@H]2CC[C@H](CNC(=O)OC(C)(C)C)CC2)C(=O)Nc2ccc3c(=O)[nH][nH]c3c2)cc1]

Tetrakis(triphenylphosphine)palladium(0), sodium carbonate and water are added to a solution of 4-bromo-N-alpha-[(trans-4-{[(tert-butoxycarbonyl)amino]methyl}cyclohexyl)carbonyl]-N-(3-oxo-2,3-dihydro-1H-indazol-6-yl)-L-phenylalaninamide and [4-(isopropylsulphamoyl)-2-methylphenyl]boronic acid in DMSO. The mixture is treated in the microwave at 110° C. for 1 to 3 hours. The mixture is filtered and purified by preparative HPLC. This gives the title compound.

### Example 50A

### tert-Butyl {[trans-4-({(2S)-3-[2′-methyl-4′-(pyrrolidin-1-ylsulphonyl)biphenyl-4-yl]-1-oxo-1-[(3-oxo-2,3-dihydro-1H-indazol-6-yl)amino]propan-2-yl}carbamoyl)cyclohexyl]methyl}carbamate

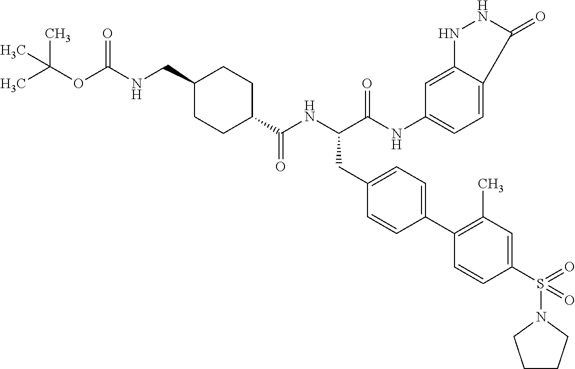 [SMILES: Cc1cc(S(=O)(=O)N2CCCC2)ccc1-c1ccc(C[C@H](NC(=O)[C@H]2CC[C@H](CNC(=O)OC(C)(C)C)CC2)C(=O)Nc2ccc3c(=O)[nH][nH]c3c2)cc1]

Tetrakis(triphenylphosphine)palladium(0), sodium carbonate and water are added to a solution of 4-bromo-N-alpha-[(trans-4-{[(tert-butoxycarbonyl)amino]methyl}cyclohexyl)carbonyl]-N-(3-oxo-2,3-dihydro-1H-indazol-6-yl)-L-phenylalaninamide and [2-methyl-4-(pyrrolidin-1-ylsulphonyl)phenyl]boronic acid in DMSO. The mixture is treated in the microwave at 110° C. for 1 to 3 hours. The mixture is filtered and purified by preparative HPLC. This gives the title compound.

### Example 51A

### tert-Butyl {[trans-4-({(2S)-3-[4′-(diethylsulphamoyl)-2′-methylbiphenyl-4-yl]-1-oxo-1-[(3-oxo-2,3-dihydro-1H-indazol-6-yl)amino]propan-2-yl}carbamoyl)cyclohexyl]methyl}carbamate

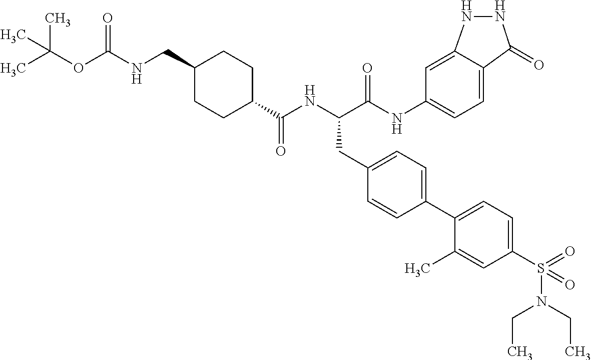 [SMILES: CCN(CC)S(=O)(=O)c1ccc(-c2ccc(C[C@H](NC(=O)[C@H]3CC[C@H](CNC(=O)OC(C)(C)C)CC3)C(=O)Nc3ccc4c(=O)[nH][nH]c4c3)cc2)c(C)c1]

Tetrakis(triphenylphosphine)palladium(0), sodium carbonate and water are added to a solution of 4-bromo-N-alpha-[(trans-4-{[(tert-butoxycarbonyl)amino]methyl}cyclohexyl)carbonyl]-N-(3-oxo-2,3-dihydro-1H-indazol-6-yl)-L-phenylalaninamide and [4-(diethyl sulphamoyl)-2-methylphenyl]boronic acid in DMSO. The mixture is treated in the microwave at 110° C. for 1 to 3 hours. The mixture is filtered and purified by preparative HPLC. This gives the title compound.

### Example 52A

### tert-Butyl {[trans-4-({(2S)-3-[2′-methyl-4′-(methylsulphamoyl)biphenyl-4-yl]-1-oxo-1-[(3-oxo-2,3-dihydro-1H-indazol-6-yl)amino]propan-2-yl}carbamoyl)cyclohexyl]methyl}carbamate

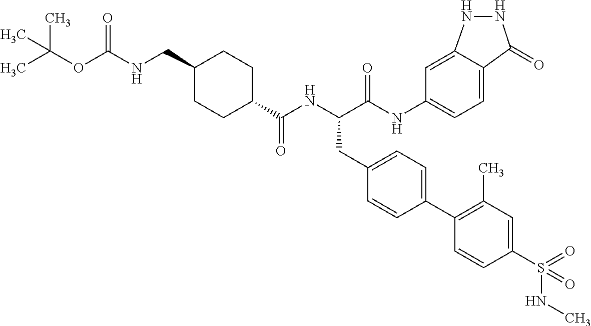 [SMILES: CNS(=O)(=O)c1ccc(-c2ccc(C[C@H](NC(=O)[C@H]3CC[C@H](CNC(=O)OC(C)(C)C)CC3)C(=O)Nc3ccc4c(=O)[nH][nH]c4c3)cc2)c(C)c1]

Tetrakis(triphenylphosphine)palladium(0), sodium carbonate and water are added to a solution of 4-bromo-N-alpha-[(trans-4-{[(tert-butoxycarbonyl)amino]methyl}cyclohexyl)carbonyl]-N-(3-oxo-2,3-dihydro-1H-indazol-6-yl)-L-phenylalaninamide and [2-methyl-4-(methylsulphamoyl)phenyl]boronic acid in DMSO. The mixture is treated in the microwave at 110° C. for 1 to 3 hours. The mixture is filtered and purified by preparative HPLC. This gives the title compound.

### WORKING EXAMPLES

General Procedure 1

10 to 20 eq. of 4M hydrogen chloride in 1,4-dioxane were added to a solution of the tert-butyl ester in 3 to 4.5 ml of 1,4-dioxane, and the mixture was stirred at RT for 4 h to 9 days. The precipitate was filtered off, washed with 1,4-dioxane or acetonitrile and then dried under high vacuum, or alternatively the crude product was separated by preparative HPLC (mobile phase: gradient of acetonitrile/water with 0.1% trifluoroacetic acid). The product-containing fractions were combined, a few drops of 4M hydrogen chloride in 1,4-dioxane were added and the mixture was concentrated on a rotary evaporator. The residue was dried under high vacuum.

### Example 1

### trans-4-(Aminomethyl)-N-[(2S)-3-[2′-methyl-4′-(piperidin-4-ylsulphamoyl)biphenyl-4-yl]-1-oxo-1-{[4-(1H-tetrazol-5-yl)phenyl]amino}propan-2-yl]cyclohexanecarboxamide hydrochloride

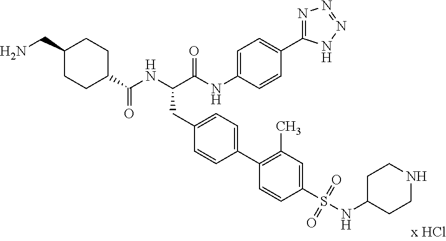

0.06 ml (0.23 mmol) of 4M hydrogen chloride in 1,4-dioxane was added to a suspension of 21 mg (0.023 mmol) of tert-butyl 4-[({4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2-methylbiphenyl-4-yl}sulphonyl)amino]piperidine-1-carboxylate in 1.0 ml of 1,4-dioxane, and the mixture was stirred at RT overnight. Subsequently, the reaction mixture was concentrated and the residue was purified chromatographically by HPLC (Method 7). This gave 3 mg (16% of theory) of the title compound.

¹H NMR (300 MHz, DMSO-d₆): δ ppm 0.75-0.96 (m, 2H), 1.07-1.41 (m, 5H), 1.49-1.61 (m, 3H), 1.62-1.79 (m, 3H), 2.05-2.17 (m, 1H), 2.23 (s, 3H), 2.56 (d, 2H), 2.81-2.96 (m, 3H), 3.00-3.12 (m, 3H), 4.65-4.77 (m, 1H), 7.27 (d, 2H), 7.30-7.40 (m, 3H), 7.56 (d, 2H), 7.60-7.66 (m, 1H), 7.67-7.71 (m, 1H), 7.84 (d, 2H), 8.11 (d, 1H), 10.04 (s, 1H), some signals obscured by solvent and water signals.

LC-MS (Method 5): Rₜ=0.67 min; MS (ESIpos): m/z=700.5 [M+H—HCl]⁺

### Example 2

### trans-4-(Aminomethyl)-N-[(2S)-1-oxo-3-[4′-(piperazin-1-yl)biphenyl-4-yl]-1-{[4-(2H-tetrazol-5-yl)phenyl]amino}propan-2-yl]cyclohexanecarboxamide hydrochloride

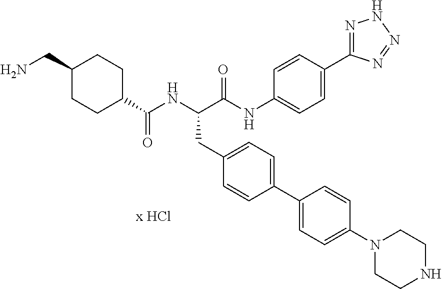

0.23 ml (0.93 mmol) of 4M hydrogen chloride in 1,4-dioxane was added to a solution of 86 mg (0.09 mmol) of tert-butyl 4-{4′-(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]biphenyl-4-yl}piperazine-1-carboxylate in 3 ml of 1,4-dioxane, and the mixture was stirred at RT for 4 h. After the addition of a further 0.23 ml (0.93 mmol) of 4M hydrogen chloride in 1,4-dioxane, the reaction mixture was stirred at RT overnight. The precipitate was filtered off, washed with 1,4-dioxane and then dried under high vacuum. The crude product was taken up in methanol, filtered through a Millipore syringe filter and separated by preparative HPLC (mobile phase: gradient of acetonitrile/water with 0.1% trifluoroacetic acid). The product-containing fractions were combined, a few drops of 4M hydrogen chloride in 1,4-dioxane were added and the mixture was concentrated on a rotary evaporator. The residue was dried under high vacuum. This gave 35 mg (55% of theory) of the title compound.

¹H-NMR (400 MHz, DMSO-d₆): δ=0.85-0.99 (m, 2H), 1.13-1.32 (m, 2H), 1.41-1.65 (m, 2H), 1.70-1.82 (m, 3H), 2.11-2.20 (m, 1H), 2.57-2.67 (m, 2H), 2.91 (dd, 1H), 3.08 (dd, 1H), 3.16-3.27 (m, 4H), 3.33-3.44 (m, 4H), 4.66-4.72 (m, 1H), 7.04 (d, 2H), 7.35 (d, 2H), 7.47-7.58 (m, 4H), 7.76-7.90 (m, 5H), 8.02 (d, 2H), 8.28 (d, 1H), 9.16 (bs, 2H), 10.60 (s, 1H).

LC-MS (Method 1): Rₜ=0.57 min; MS (ESIpos): m/z=606 [M+H—HCl]⁺

### Example 3

### trans-4-(Aminomethyl)-N-[(2S)-3-[4′-(2-methoxyethoxy)biphenyl-4-yl]-1-oxo-1-{[4-(2H-tetrazol-5-yl)phenyl]amino}propan-2-yl]cyclohexanecarboxamide hydrochloride

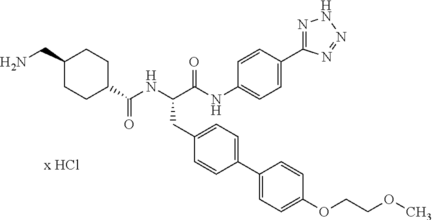

According to General Procedure 1, tert-butyl [(trans-4-{[(2S)-3-[4′-(2-methoxyethoxy)biphenyl-4-yl]-1-oxo-1-{[4-(2H-tetrazol-5-yl)phenyl]amino}propan-2-yl]carbamoyl}cyclohexyl)methyl]carbamate (80 mg, 0.12 mmol) was converted into the title compound. This gave 68 mg (87% of theory).

¹H-NMR (400 MHz, DMSO-d₆): δ=0.84-0.98 (m, 2H), 1.13-1.34 (m, 2H), 1.39-1.64 (m, 2H), 1.66-1.80 (m, 3H), 2.08-2.19 (m, 1H), 2.57-2.69 (m, 2H), 2.91 (dd, 1H), 3.09 (dd, 1H), 3.31 (s, 3H), 3.63-3.68 (m, 2H), 4.07-4.14 (m, 2H), 4.66-4.74 (m, 1H), 6.99 (d, 2H), 7.34 (d, 2H), 7.45-7.60 (m, 4H), 7.65-7.85 (m, 5H), 8.01 (d, 2H), 8.23 (d, 1H), 10.54 (s, 1H).

LC-MS (Method 1): Rₜ=0.79 min; MS (ESIpos): m/z=596 [M+H—HCl]⁺

### Example 4

### trans-4-(Aminomethyl)-N-[(2S)-3-[4′-(4-methylpiperazin-1-yl)biphenyl-4-yl]-1-oxo-1-{[4-(2H-tetrazol-5-yl)phenyl]amino}propan-2-yl]cyclohexanecarboxamide hydrochloride

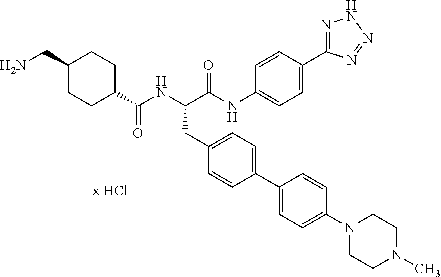

According to General Procedure 1, tert-butyl [(trans-4-{[(2S)-3-[4′-(4-methylpiperazin-1-yl)biphenyl-4-yl]-1-oxo-1-{[4-(2H-tetrazol-5-yl)phenyl]amino}propan-2-yl]carbamoyl}cyclohexyl)methyl]carbamate trifluoroacetate (108 mg, 0.13 mmol) was converted into the title compound. This gave 82 mg (88% of theory).

¹H-NMR (400 MHz, DMSO-d₆): δ=0.85-1.00 (m, 2H), 1.14-1.33 (m, 2H), 1.40-1.66 (m, 2H), 1.68-1.82 (m, 3H), 2.10-2.20 (m, 1H), 2.58-2.69 (m, 2H), 2.82 (d, 3H), 2.91 (dd, 1H), 3.03-3.21 (m, 5H), 3.48 (d, 2H), 3.87 (d, 2H), 4.66-4.73 (m, 1H), 7.07 (d, 2H), 7.36 (d, 2H), 7.50-7.60 (m, 4H), 7.72-7.96 (m, 4H), 8.02 (d, 2H), 8.27 (d, 1H), 10.56-10.70 (m, 2H).

LC-MS (Method 1): Rₜ=0.56 min; MS (ESIpos): m/z=620 [M+H—HCl]⁺

### Example 5

### trans-4-(Aminomethyl)-N-[(2S)-3-[4′-(morpholin-4-yl)biphenyl-4-yl]-1-oxo-1-{[4-(2H-tetrazol-5-yl)phenyl]amino}propan-2-yl]cyclohexanecarboxamide hydrochloride

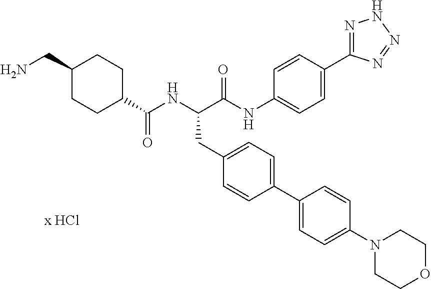

According to General Procedure 1, 54 mg (0.07 mmol) of tert-butyl [(trans-4-{[(2S)-3-[4′-(morpholin-4-yl)biphenyl-4-yl]-1-oxo-1-{[4-(2H-tetrazol-5-yl)phenyl]amino}propan-2-yl]carbamoyl}cyclohexyl)methyl]carbamate hydrochloride were converted into the title compound. This gave 34 mg (81% of theory).

¹H-NMR (400 MHz, DMSO-d₆): δ=0.85-0.99 (m, 2H), 1.12-1.32 (m, 2H), 1.40-1.64 (m, 2H), 1.69-1.81 (m, 3H), 2.10-2.20 (m, 1H), 2.58-2.69 (m, 2H), 2.91 (dd, 1H), 3.08 (dd, 1H), 3.13-3.22 (m, 4H), 3.74-3.81 (m, 4H), 4.65-4.74 (m, 1H), 7.07 (d, 2H), 7.35 (d, 2H), 7.49-7.58 (m, 4H), 7.75-7.90 (m, 5H), 8.02 (d, 2H), 8.27 (d, 1H), 10.60 (s, 1H).

LC-MS (Method 1): Rₜ=0.76 min; MS (ESIpos): m/z=607 [M+H—HCl]⁺

### Example 6

### trans-4-(Aminomethyl)-N-{(2S)-1-oxo-1-{[4-(5-oxo-4,5-dihydro-1,2,4-oxadiazol-3-yl)phenyl]amino}-3-[4′-(piperazin-1-yl)biphenyl-4-yl]propan-2-yl}cyclohexanecarboxamide hydrochloride

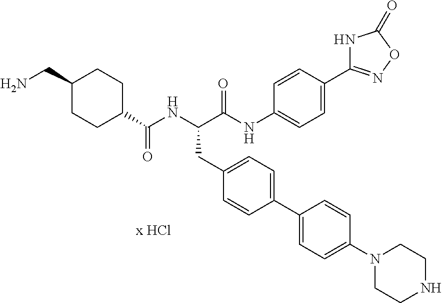

According to General Procedure 1, 16 mg (0.02 mmol) of tert-butyl 4-{4′-[(2S)-2-{[(trans-4-{[(tert-butoxycarbonyl)amino]methyl}cyclohexyl)carbonyl]amino}-3-oxo-3-{[4-(5-oxo-4,5-dihydro-1,2,4-oxadiazol-3-yl)phenyl]amino}propyl]biphenyl-4-yl}piperazine-1-carboxylate were reacted to give the title compound. This gave 7 mg (56% of theory).

¹H-NMR (400 MHz, DMSO-d₆): δ=0.84-0.98 (m, 2H), 1.13-1.33 (m, 2H), 1.41-1.65 (m, 2H), 1.70-1.81 (m, 3H), 2.10-2.20 (m, 1H), 2.57-2.68 (m, 2H), 2.91 (dd, 1H), 3.07 (dd, 1H), 3.18-3.27 (m, 4H), 3.36-3.45 (m, 4H), 4.65-4.72 (m, 1H), 7.05 (d, 2H), 7.34 (d, 2H), 7.50-7.58 (m, 4H), 7.74-7.89 (m, 6H), 8.27 (d, 1H), 9.11 (bs, 1H), 10.62 (s, 1H), 12.93 (s, 1H).

LC-MS (Method 1): Rₜ=0.61 min; MS (ESIpos): m/z=622 [M+H—HCl]⁺

### Example 7

### Methyl 4′-[(2S)-2-({[trans-4-(aminomethyl)cyclohexyl]carbonyl}amino)-3-oxo-3-{[4-(2H-tetrazol-5-yl)phenyl]amino}propyl]biphenyl-4-carboxylic acid hydrochloride

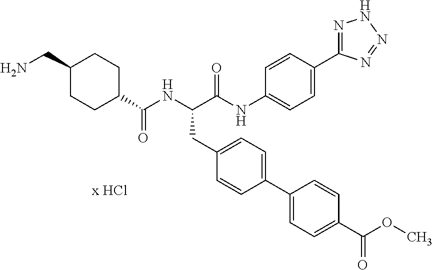

0.24 ml (0.94 mmol) of 4M hydrogen chloride in 1,4-dioxane was added to a solution of 50 mg (0.06 mmol) of methyl 4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]biphenyl-4-carboxylate in 2 ml of 1,4-dioxane, and the mixture was stirred at RT overnight. After the addition of a further 0.20 ml (0.79 mmol) of 4M hydrogen chloride in 1,4-dioxane, the mixture was stirred at RT overnight. The precipitate was filtered off, washed with a little acetonitrile and dried under high vacuum. This gave 40 mg (94% of theory) of the title compound.

¹H NMR (400 MHz, DMSO-d₆) δ=0.83-1.00 (m, 2H), 1.10-1.34 (m, 2H), 1.39-1.65 (m, 2H), 1.67-1.82 (m, 3H), 2.09-2.22 (m, 1H), 2.58-2.68 (m, 2H), 2.88-3.01 (m, 1H), 3.07-3.18 (m, 1H), 3.87 (s, 3H), 4.68-4.79 (m, 1H), 7.45 (d, 2H), 7.63-7.89 (m, 9H), 7.97-8.08 (m, 4H), 8.27 (d, 1H), 10.58 (s, 1H)

LC-MS (Method 1): Rₜ=0.75 min; MS (ESIpos): m/z=580 [M+H—HCl]⁺

### Example 8

### trans-4-(Aminomethyl)-N-[(2S)-1-oxo-3-[4′-(piperidin-4-ylsulphamoyl)-2′-(trifluoromethyl)biphenyl-4-yl]-1-{[4-(2H-tetrazol-5-yl)phenyl]amino}propan-2-yl]cyclohexanecarboxamide hydrochloride

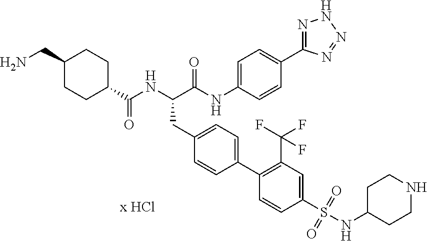 [SMILES: NC[C@H]1CC[C@H](C(=O)N[C@@H](Cc2ccc(-c3ccc(S(=O)(=O)NC4CCNCC4)cc3C(F)(F)F)cc2)C(=O)Nc2ccc(-c3nn[nH]n3)cc2)CC1]

To a solution of 28.4 mg (0.03 mmol) of tert-butyl 4-[({4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2-(trifluoromethyl)biphenyl-4-yl}sulphonyl)amino]piperidine-1-carboxylate in 1.5 ml of dioxane was added 0.1 ml (0.44 mmol) of 4M hydrogen chloride in dioxane. The mixture was stirred at RT for 9 days. The precipitated product was filtered off with suction, taken up in 1 ml of DMF and separated by preparative HPLC (mobile phase: gradient of acetonitrile/water with 0.1% trifluoroacetic acid). The product-containing fractions were combined and concentrated on a rotary evaporator. The residue was dried under high vacuum. This gave 19 mg (76% of theory) of the title compound.

LC-MS (Method 1): Rₜ=0.63 min; MS (ESIpos): m/z=754 [M+H—HCl]⁺

¹H NMR (400 MHz, DMSO-d₆) δ=0.85-1.00 (m, 2H), 1.11-1.35 (m, 3H), 1.42-1.53 (m, 1H), 1.55-1.63 (m, 3H), 1.76 (m, 5H), 2.10-2.20 (m, 1H), 2.60-2.68 (m, 2H), 2.83-3.02 (m, 3H), 3.17 (m, 3H), 3.36-3.49 (m, 2H), 4.72-4.81 (m, 1H), 7.28 (d, 2H), 7.43 (d, 2H), 7.60 (d, 1H), 7.76-7.86 (m, 4H), 8.02 (d, 2H), 8.12 (d, 1H), 8.19 (s, 1H), 8.30 (d, 1H), 8.34 (d, 1H), 8.55 (br. s, 1H), 8.73 (br. s, 1H), 10.57 (s, 1H).

### Example 9

### Glycylglycyl-N-{4′-[(2S)-2-({[trans-4-(aminomethyl)cyclohexyl]carbonyl}amino)-3-oxo-3-{[4-(2H-tetrazol-5-yl)phenyl]amino}propyl]-2-methylbiphenyl-4-yl}glycinamide hydrochloride

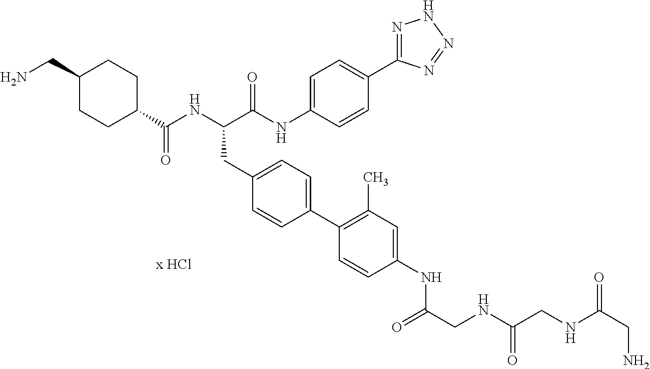 [SMILES: Cc1cc(NC(=O)CNC(=O)CNC(=O)CN)ccc1-c1ccc(C[C@H](NC(=O)[C@H]2CC[C@H](CN)CC2)C(=O)Nc2ccc(-c3nn[nH]n3)cc2)cc1]

0.5 ml (2.00 mmol) of 4M hydrogen chloride in 1,4-dioxane was added to a solution of 60 mg (0.06 mmol) of N-(tert-butoxycarbonyl)glycylglycyl-N-{4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2-methylbiphenyl-4-yl}glycinamide in 0.8 ml of tetrahydrofuran, and the mixture was stirred at RT for 16 h and the solvent was removed. The solid obtained was dissolved in 0.5 ml of dioxane, 0.5 ml (2.00 mmol) of 4M hydrogen chloride in 1,4-dioxane was added and the mixture was stirred at RT for 16 h. The solid formed was washed with acetonitrile/ethanol (4:1 to 3:1) and dried under high vacuum. 16 mg (29% of theory, 88% purity) of the title compound were obtained.

¹H NMR (400 MHz, DMSO-d₆) δ=0.75-1.00 (m, 2H), 1.08-1.36 (m, 2H), 1.40-1.62 (m, 2H), 1.66-1.84 (m, 4H), 2.02-2.26 (m, 4H), 2.58-2.72 (m, 2H), 2.96 (m, 1H), 3.12 (m, 1H), 3.52-3.69 (m, 2H), 3.91 (m, 4H), 4.60-4.87 (m, 1H), 7.10 (d, 1H), 7.23 (d, 1H), 7.36 (d, 2H), 7.44-7.57 (m, 2H), 7.71-8.20 (m, 11H), 8.24-8.33 (m, 1H), 8.43 (m, 1H), 8.74 (m, 1H), 10.01 (br. s., 1H), 10.55 (br. s., 1H).

LC-MS (Method 1): Rₜ=0.57 min; MS (ESIpos): m/z=722 [M+H—HCl]⁺

### Example 10

### trans-4-(Aminomethyl)-N-[(2S)-3-(2′-methyl-4′-{[(2-methylpiperidin-4-yl]sulphamoyl}biphenyl-4-yl)-1-oxo-1-{[4-(2H-tetrazol-5-yl)phenyl]amino}propan-2-yl]cyclohexanecarboxamide hydrochloride

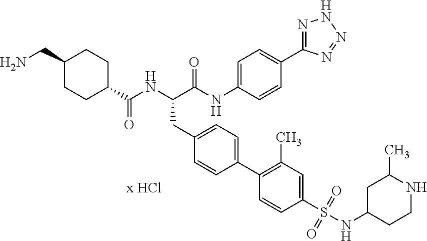 [SMILES: Cc1cc(S(=O)(=O)NC2CCNC(C)C2)ccc1-c1ccc(C[C@H](NC(=O)[C@H]2CC[C@H](CN)CC2)C(=O)Nc2ccc(-c3nn[nH]n3)cc2)cc1]

0.4 ml (1.6 mmol) of 4M hydrogen chloride in dioxane was added to a solution of 99 mg (0.1 mmol) of tert-butyl 4-[({4′-[(2S)-2-{[(trans-4-{[(tert-butoxycarbonyl)amino]methyl}cyclohexyl)carbonyl]amino}-3-oxo-3-{[4-(2H-tetrazol-5-yl)-phenyl]amino}propyl]-2-methylbiphenyl-4-yl}sulphonyl)amino]-2-methylpiperidine-1-carboxylate in 4.5 ml of dioxane. The mixture was stirred at RT for 10 days. The precipitated product was filtered off with suction, taken up in 1 ml of DMF and separated by preparative HPLC (mobile phase: gradient of acetonitrile/water with 0.1% trifluoroacetic acid). The product-containing fractions were combined and concentrated on a rotary evaporator. The residue was dried under high vacuum. 58 mg (85% of theory, 90% purity) of the title compound were obtained.

LC-MS (Method 1): Rₜ=0.60 min; MS (ESIpos): m/z=714 [M+H—HCl]⁺

¹H NMR (400 MHz, DMSO-d₆) δ ppm 0.82-1.01 (m, 2H), 1.18 (d, 3H), 1.21-1.63 (m, 5H), 1.74 (m, 4H), 1.82-1.90 (m, 1H), 2.11-2.20 (m, 1H), 2.26 (s, 3H), 2.63 (t, 2H), 2.80-2.92 (m, 1H), 2.92-3.01 (m, 1H), 3.08-3.22 (m, 3H), 3.29-3.41 (m, 1H), 4.59-4.84 (m, 1H), 7.29 (d, 2H), 7.34-7.45 (m, 3H), 7.63-7.76 (m, 2H), 7.84 (m, 5H), 7.96-8.08 (m, 3H), 8.32 (d, 1H), 8.50-8.72 (m, 1H), 9.00 (br. d, 1H), 10.58 (s, 1H).

### Example 11

### Methyl 4′-[(2S)-2-({[trans-4-(aminomethyl)cyclohexyl]carbonyl}amino)-3-oxo-3-{[4-(2H-tetrazol-5-yl)phenyl]amino}propyl]-2-chlorobiphenyl-4-carboxylate hydrochloride

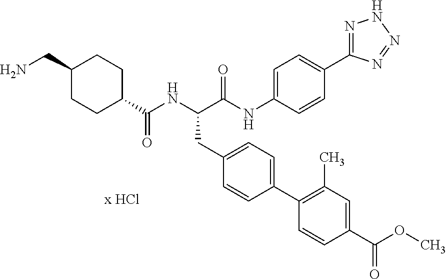 [SMILES: COC(=O)c1ccc(-c2ccc(C[C@H](NC(=O)[C@H]3CC[C@H](CN)CC3)C(=O)Nc3ccc(-c4nn[nH]n4)cc3)cc2)c(C)c1]

To a solution of 26 mg (36 μmol) of methyl 4′-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]-2-chlorobiphenyl-4-carboxylate in 2 ml of dioxane were added 136 μl (0.55 mmol) of 4M hydrogen chloride in dioxane. The mixture was stirred at RT for 4 days. The precipitated product was filtered off with suction, washed with acetonitrile and dried under high vacuum. 22 mg (81% of theory, 93% purity) of the title compound were obtained.

LC-MS (Method 1): Rₜ=0.79 min; MS (ESIpos): m/z=616 [M+H—HCl]⁺

¹H NMR (400 MHz, DMSO-d₆) δ ppm 0.87-0.98 (m, 2H), 1.11-1.34 (m, 2H), 1.40-1.52 (m, 1H), 1.54-1.63 (m, 1H), 1.65-1.82 (m, 3H), 2.10-2.22 (m, 1H), 2.63 (t, 2H), 2.89-3.01 (m, 1H), 3.16 (dd, 1H), 3.90 (s, 3H), 4.71-4.81 (m, 1H), 7.41 (d, 2H), 7.43 (d, 2H), 7.53 (d, 1H), 7.75 (br. s, 3H), 7.84 (d, 2H), 7.96 (dd, 1H), 8.00-8.05 (m, 3H), 8.28 (d, 1H), 10.55 (s, 1H).

### Example 12

### trans-4-(Aminomethyl)-N-{(2S)-3-[4′-(cyclopropylsulphamoyl)-2′-methylbiphenyl-4-yl]-1-oxo-1-[(3-oxo-2,3-dihydro-1H-indazol-6-yl)amino]propan-2-yl}cyclohexanecarboxamide hydrochloride

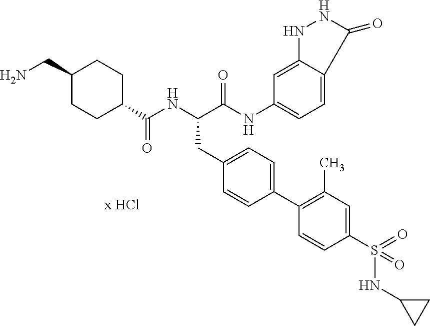 [SMILES: Cc1cc(S(=O)(=O)NC2CC2)ccc1-c1ccc(C[C@H](NC(=O)[C@H]2CC[C@H](CN)CC2)C(=O)Nc2ccc3c(=O)[nH][nH]c3c2)cc1]

98 μl (0.39 mmol) of 4M hydrogen chloride in dioxane were added to a solution of 58.5 mg (78.5 μmol) of tert-butyl {[trans-4-({(2S)-3-[4′-(cyclopropylsulphamoyl)-2′-methylbiphenyl-4-yl]-1-oxo-1-[(3-oxo-2,3-dihydro-1H-indazol-6-yl)amino]propan-2-yl}carbamoyl)cyclohexyl]methyl}carbamate in 2 ml of dichloromethane. The mixture was stirred at RT overnight. Acetonitrile was added to the suspension, and the precipitated solid was filtered off with suction and washed with acetonitrile. The product was then dried under high vacuum. This gave 50 mg (92% of theory) of the title compound.

LC-MS (Method 4): Rₜ=0.77 min; MS (ESIpos): m/z=645 [M+H—HCl]⁺

¹H NMR (400 MHz, DMSO-d₆) δ ppm 0.38-0.45 (m, 2H), 0.46-0.54 (m, 2H), 0.80-0.97 (m, 2H), 1.05-1.33 (m, 2H), 1.36-1.49 (m, 1H), 1.53-1.62 (m, 1H), 1.65-1.82 (m, 3H), 2.07-2.15 (m, 2H), 2.26 (s, 3H), 2.30-2.33 (m, 1H), 2.58-2.69 (m, 3H), 2.92 (dd, 1H), 3.11 (dd, 1H), 4.36-4.82 (m, 1H), 6.82 (d, 1H), 7.01 (dd, 1H), 7.29 (d, 2H), 7.35-7.40 (m, 3H), 7.42 (d, 1H), 7.66 (dd, 1H), 7.71 (m, 2H), 7.90 (d, 1H), 8.07-8.17 (m, 1H), 9.96 (s, 1H), 10.47 (s, 1H), 10.53 (s, 1H).

### Example 13

### trans-4-(Aminomethyl)-N-{(2S)-3-[4′-(dimethylsulphamoyl)-2′-methylbiphenyl-4-yl]-1-oxo-1-[(3-oxo-2,3-dihydro-1H-indazol-6-yl)amino]propan-2-yl}cyclohexanecarboxamide hydrochloride

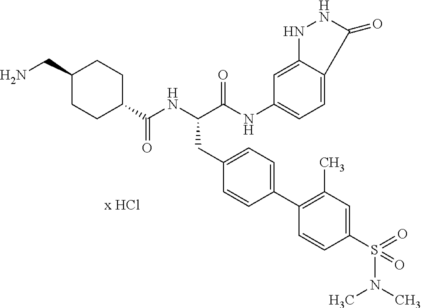 [SMILES: Cc1cc(S(=O)(=O)N(C)C)ccc1-c1ccc(C[C@H](NC(=O)[C@H]2CC[C@H](CN)CC2)C(=O)Nc2ccc3c(=O)[nH][nH]c3c2)cc1]

113 μl (0.45 mmol) of 4M hydrogen chloride in dioxane were added to a solution of 66.3 mg (90.5 μmol) of tert-butyl {[trans-4-({(2S)-3-[4′-(dimethylsulphamoyl)-2′-methylbiphenyl-4-yl]-1-oxo-1-[(3-oxo-2,3-dihydro-1H-indazol-6-yl)amino]propan-2-yl}carbamoyl)cyclohexyl]methyl}carbamate in 2 ml of dichloromethane. The mixture was stirred at RT overnight. Acetonitrile was added to the suspension, and the precipitated solid was filtered off with suction and washed with acetonitrile. The product was then dried under high vacuum. This gave 54 mg (89% of theory) of the title compound.

LC-MS (Method 4): Rₜ=0.79 min; MS (ESIpos): m/z=633 [M+H—HCl]⁺

¹H NMR (300 MHz, DMSO-d₆) δ ppm 0.80-1.00 (m, 2H), 1.07-1.37 (m, 2H), 1.39-1.51 (m, 1H), 1.52-1.62 (m, 1H), 1.64-1.82 (m, 3H), 2.07-2.20 (m, 1H), 2.28 (s, 3H), 2.65 (s, 8H), 2.90 (dd, 1H), 3.08 (dd, 1H), 4.54-4.80 (m, 1H), 6.82 (d, 1H), 7.01 (dd, 1H), 7.29 (d, 2H), 7.35-7.44 (m, 4H), 7.60 (d, 1H), 7.65 (s, 1H), 7.73 (br. s, 2H), 8.14 (d, 1H), 9.97 (s, 1H), 10.46 (s, 1H), 10.54 (s, 1H).

### Example 14

### trans-4-(Aminomethyl)-N-{(2S)-3-[4′-(isopropylsulphamoyl)-2′-methylbiphenyl-4-yl]-1-oxo-1-[(3-oxo-2,3-dihydro-1H-indazol-6-yl)amino]propan-2-yl}cyclohexanecarboxamide hydrochloride

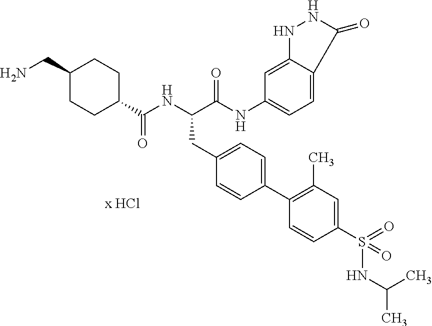 [SMILES: Cc1cc(S(=O)(=O)NC(C)C)ccc1-c1ccc(C[C@H](NC(=O)[C@H]2CC[C@H](CN)CC2)C(=O)Nc2ccc3c(=O)[nH][nH]c3c2)cc1]

87 μl (0.35 mmol) of 4M hydrogen chloride in dioxane were added to a solution of 52.1 mg (69.8 μmol) of tert-butyl {[trans-4-({(2S)-3-[4′-(isopropylsulphamoyl)-2′-methylbiphenyl-4-yl]-1-oxo-1-[(3-oxo-2,3-dihydro-1H-indazol-6-yl)amino]propan-2-yl}carbamoyl)cyclohexyl]methyl}carbamate in 2 ml of dichloromethane. The mixture was stirred at RT overnight. Acetonitrile was added to the suspension, and the precipitated solid was filtered off with suction and washed with acetonitrile. The product was then dried under high vacuum. This gave 43 mg (90% of theory) of the title compound.

LC-MS (Method 4): Rₜ=0.78 min; MS (ESIpos): m/z=645 [M+H—HCl]⁺

¹H NMR (400 MHz, DMSO-d₆) δ ppm 0.85-0.94 (m, 2H), 0.98 (d, 6H), 1.10-1.33 (m, 2H), 1.39-1.50 (m, 1H), 1.52-1.61 (m, 1H), 1.67-1.79 (m, 2H), 2.13 (t, 1H), 2.25 (s, 3H), 2.62 (t, 2H), 2.91 (dd, 1H), 3.09 (dd, 1H), 3.23-3.30 (m, 1H), 4.49-4.88 (m, 1H), 6.83 (d, 1H), 7.01 (dd, 1H), 7.27 (d, 2H), 7.32-7.40 (m, 3H), 7.42 (d, 1H), 7.56 (d, 1H), 7.64 (dd, 1H), 7.70 (d, 1H), 7.76 (br. s, 2H), 8.14 (d, 1H), 9.99 (s, 1H), 10.48 (s, 1H), 10.54 (s, 1H).

### Example 15

### trans-4-(Aminomethyl)-N-{(2S)-3-[2′-methyl-4′-(pyrrolidin-1-ylsulphonyl)biphenyl-4-yl]-1-oxo-1-[(3-oxo-2,3-dihydro-1H-indazol-6-yl)amino]propan-2-yl}cyclohexanecarboxamide hydrochloride

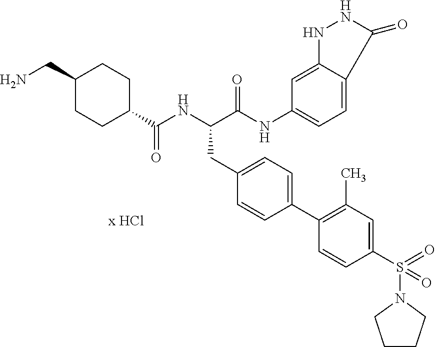 [SMILES: Cc1cc(S(=O)(=O)N2CCCC2)ccc1-c1ccc(C[C@H](NC(=O)[C@H]2CC[C@H](CN)CC2)C(=O)Nc2ccc3c(=O)[nH][nH]c3c2)cc1]

100 μl (0.40 mmol) of 4M hydrogen chloride in dioxane were added to a solution of 61.0 mg (80.4 μmol) of tert-butyl {[trans-4-({(2S)-3-[2′-methyl-4′-(pyrrolidin-1-ylsulphonyl)biphenyl-4-yl]-1-oxo-1-[(3-oxo-2,3-dihydro-1H-indazol-6-yl)amino]propan-2-yl}carbamoyl)cyclohexyl]methyl}carbamate in 2 ml of dichloromethane. The mixture was stirred at RT overnight. Acetonitrile was added to the suspension, and the precipitated solid was filtered off with suction and washed with acetonitrile. The product was then dried under high vacuum. This gave 50 mg (88% of theory) of the title compound.

LC-MS (Method 4): Rₜ=0.84 min; MS (ESIpos): m/z=660 [M+H—HCl]⁺

¹H NMR (300 MHz, DMSO-d₆) δ ppm 0.81-0.98 (m, 2H), 1.05-1.32 (m, 2H), 1.40-1.50 (m, 1H), 1.50-1.61 (m, 1H), 1.64-1.79 (m, 7H), 2.07 (t, 1H), 2.28 (s, 3H), 2.63 (t, 2H), 2.91 (dd, 1H), 3.08 (dd, 1H), 3.12-3.22 (m, 4H), 4.58-4.79 (m, 1H), 6.84 (d, 1H), 7.01 (dd, 1H), 7.28 (d, 2H), 7.35-7.44 (m, 4H), 7.65 (d, 1H), 7.70 (s, 1H), 7.75 (br. s, 2H), 8.18 (d, 1H), 10.01 (s, 1H), 10.49-10.52 (m, 1H), 10.56 (s, 1H).

### Example 16

### trans-4-(Aminomethyl)-N-[(2S)-3-{2′-methyl-4′-[(3R)-pyrrolidin-3-ylsulphamoyl]biphenyl-4-yl}-1-oxo-1-{[4-(2H-tetrazol-5-yl)phenyl]amino}propan-2-yl]cyclohexanecarboxamide hydrochloride

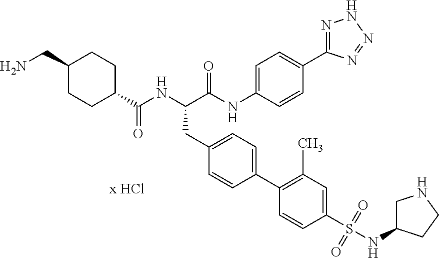 [SMILES: Cc1cc(S(=O)(=O)N[C@@H]2CCNC2)ccc1-c1ccc(C[C@H](NC(=O)[C@H]2CC[C@H](CN)CC2)C(=O)Nc2ccc(-c3nn[nH]n3)cc2)cc1]

487 μl (1.95 mmol) of 4M hydrogen chloride in dioxane were added to a solution of 115.1 mg (130 μmol) of tert-butyl (3R)-3-[({4′-[(2S)-2-{[(trans-4-{[(tert-butoxycarbonyl)amino]methyl}cyclohexyl)carbonyl]amino}-3-oxo-3-{[4-(2H-tetrazol-5-yl)-phenyl]amino}propyl]-2-methylbiphenyl-4-yl}sulphonyl)amino]pyrrolidine-1-carboxylate in 5 ml of dioxane. The mixture was stirred at RT for 10 days. The precipitated product was filtered off with suction, washed with a little dioxane and dried under high vacuum. The residue was taken up in 1 ml of DMF and 3 ml of acetonitrile and separated by preparative HPLC (mobile phase: gradient of acetonitrile/water with 0.1% trifluoroacetic acid). The product-containing test tubes were concentrated and dried under high vacuum. The residue was dissolved in methanol, and 0.1 ml of 4N hydrogen chloride in dioxane was added prior to concentration. The solid was dried under high vacuum. This gave 64 mg (64% of theory) of the title compound.

LCMS (Method 1): Rₜ=0.58 min; MS (ESIpos): m/z=686 [M+H—HCl]⁺

¹H NMR (400 MHz, DMSO-d₆) δ ppm 0.93 (d, 2H), 1.05-1.37 (m, 2H), 1.41-1.52 (m, 1H), 1.57 (d, 1H), 1.67-1.85 (m, 4H), 1.97 (s, 1H), 2.16 (br. s., 1H), 2.27 (s, 3H), 2.63 (t, 2H), 2.88-3.07 (m, 2H), 3.09-3.26 (m, 5H), 4.67-4.84 (m, 1H), 7.30 (d, 2H), 7.38-7.45 (m, 3H), 7.71 (d, 1H), 7.76 (s, 1H), 7.84 (d, 2H), 7.86-7.90 (m, 2H), 8.03 (d, 2H), 8.20 (d, 1H), 8.32 (d, 1H), 8.94-9.39 (m, 2H), 10.59 (br. s., 1H).

### Example 17

### trans-4-(Aminomethyl)-N-{(2S)-3-[4′-(diethylsulphamoyl)-2′-methylbiphenyl-4-yl]-1-oxo-1-[(3-oxo-2,3-dihydro-1H-indazol-6-yl)amino]propan-2-yl}cyclohexanecarboxamide hydrochloride

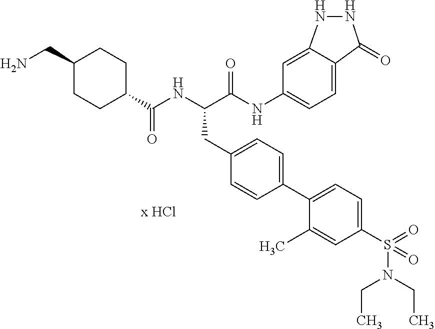 [SMILES: CCN(CC)S(=O)(=O)c1ccc(-c2ccc(C[C@H](NC(=O)[C@H]3CC[C@H](CN)CC3)C(=O)Nc3ccc4c(=O)[nH][nH]c4c3)cc2)c(C)c1]

106 μl (0.43 mmol) of 4M hydrogen chloride in dioxane were added to a solution of 64.7 mg (85.0 μmol) of tert-butyl {[trans-4-({(2S)-3-[4′-(diethylsulphamoyl)-2′-methylbiphenyl-4-yl]-1-oxo-1-[(3-oxo-2,3-dihydro-1H-indazol-6-yl)amino]propan-2-yl}carbamoyl)cyclohexyl]methyl}carbamate in 2.3 ml of dichloromethane. The mixture was stirred at RT overnight. Acetonitrile was added to the suspension, and the precipitated solid was filtered off with suction and washed with acetonitrile. The product was then dried under high vacuum. This gave 44 mg (72% of theory) of the title compound.

LC-MS (Method 4): Rₜ=0.91 min; MS (ESIpos): m/z=661 [M+H—HCl]⁺

¹H NMR (300 MHz, DMSO-d₆) δ ppm 0.79-0.98 (m, 2H), 1.07 (t, 6H), 1.11-1.34 (m, 2H), 1.41-1.51 (m, 1H), 1.51-1.62 (m, 1H), 1.64-1.80 (m, 3H), 2.08-2.19 (m, 1H), 2.26 (s, 3H), 2.58-2.66 (m, 2H), 2.84-2.98 (m, 1H), 3.08 (dd, 1H), 3.18 (q, 4H), 4.58-4.85 (m, 1H), 6.82 (d, 1H), 7.02 (dd, 1H), 7.27 (d, 2H), 7.32-7.45 (m, 4H), 7.62 (d, 1H), 7.69 (s, 1H), 7.77 (br. s, 2H), 8.17 (d, 1H), 9.98 (s, 1H), 10.50 (d, 2H).

### Example 18

### trans-4-(Aminomethyl)-N-{(2S)-3-[2′-methyl-4′-(methylsulphamoyl)biphenyl-4-yl]-1-oxo-1-[(3-oxo-2,3-dihydro-1H-indazol-6-yl)amino]propan-2-yl}cyclohexanecarboxamide hydrochloride

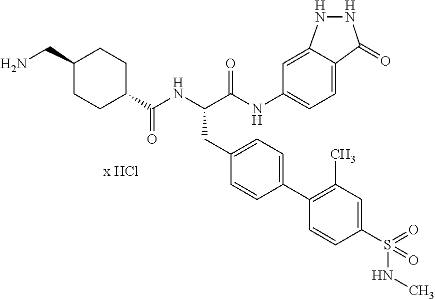 [SMILES: CNS(=O)(=O)c1ccc(-c2ccc(C[C@H](NC(=O)[C@H]3CC[C@H](CN)CC3)C(=O)Nc3ccc4c(=O)[nH][nH]c4c3)cc2)c(C)c1]

42 μl (0.17 mmol) of 4M hydrogen chloride in dioxane were added to a solution of 24.2 mg (33.7 μmol) of tert-butyl {[trans4-({(2S)-3-[2′-methyl-4′-(methylsulphamoyl)biphenyl-4-yl]-1-oxo-1-[(3-oxo-2,3-dihydro-1H-indazol-6-yl)amino]propan-2-yl}carbamoyl)cyclohexyl]methyl}carbamate in 1 ml of dichloromethane. The mixture was stirred at RT overnight. Acetonitrile was added to the suspension, and the precipitated solid was filtered off with suction and washed with acetonitrile. The product was then dried under high vacuum. This gave 13 mg (57% of theory) of the title compound.

LC-MS (Method 4): Rₜ=0.74 min; MS (ESIpos): m/z=619 [M+H—HCl]⁺

¹H NMR (300 MHz, DMSO-d₆) δ ppm 0.80-0.99 (m, 2H), 1.06-1.32 (m, 2H), 1.36-1.50 (m, 1H), 1.51-1.60 (m, 1H), 1.63-1.84 (m, 3H), 2.13 (t, 1H), 2.26 (s, 3H), 2.44 (d, 3H), 2.63 (t, 2H), 2.84-2.97 (m, 1H), 3.11 (dd, 1H), 4.56-4.79 (m, 1H), 6.81 (d, 1H), 7.02 (dd, 1H), 7.29 (d, 2H), 7.39 (m, 5H), 7.60-7.65 (m, 1H), 7.68 (m, 3H), 8.13 (d, 1H), 9.95 (s, 1H), 10.45 (br. s, 1H), 10.54 (br. s, 1H).

### Example 19

### trans-4-(Aminomethyl)-N-[(2S)-1-{[4-(5-chloro-4H-1,2,4-triazol-3-yl)phenyl]amino}-3-{4′-[(2-hydroxyethyl)sulphamoyl]biphenyl-4-yl}-1-oxopropan-2-yl]cyclohexanecarboxamide hydrochloride

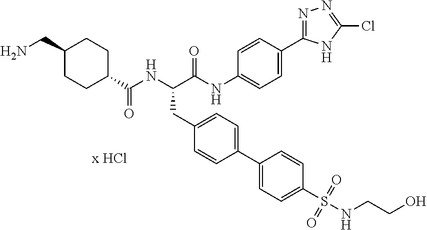 [SMILES: NC[C@H]1CC[C@H](C(=O)N[C@@H](Cc2ccc(-c3ccc(S(=O)(=O)NCCO)cc3)cc2)C(=O)Nc2ccc(-c3nnc(Cl)[nH]3)cc2)CC1]

47 μl (0.19 mmol) of 4M hydrogen chloride in dioxane were added to a solution of 20.8 mg (26.7 μmol) of tert-butyl [(trans-4-{[(2S)-1-{[4-(3-chloro-4H-1,2,4-triazol-5-yl)phenyl]amino}-3-{4′-[(2-hydroxyethyl)sulphamoyl]biphenyl-4-yl}-1-oxopropan-2-yl]carbamoyl}cyclohexyl)methyl]carbamate in 1 ml of dichloromethane. The mixture was stirred at RT overnight. The mixture was stirred at 40° C. for 2 h, 5 eq. of 4M hydrogen chloride in dioxane were added and the mixture was then stirred overnight again. Acetonitrile was added to the suspension, and the precipitated solid was filtered off with suction and washed with acetonitrile. The product was then dried under high vacuum. This gave 11 mg (55% of theory) of the title compound.

LC-MS (Method 4): Rₜ=0.78 min; MS (ESIpos): m/z=680 [M+H—HCl]⁺

¹H NMR (300 MHz, DMSO-d₆) δ ppm 0.72-0.96 (m, 2H), 1.04-1.43 (m, 4H), 1.49-1.59 (m, 1H), 1.61-1.79 (m, 3H), 2.04-2.16 (m, 1H), 2.21-2.28 (m, 1H), 2.65-2.71 (m, 1H), 2.74-2.80 (m, 2H), 2.84-2.95 (m, 1H), 4.54-4.77 (m, 1H), 7.35-7.43 (m, 2H), 7.57-7.68 (m, 4H), 7.76-7.88 (m, 5H), 8.14-8.24 (m, 1H), 8.34 (s, 1H), 10.28 (s, 1H).

### Example 20

### trans-4-(Aminomethyl)-N-[(2S)-1-{[4-(3-chloro-4H-1,2,4-triazol-5-yl)phenyl]amino}-3-(2′-methyl-4′-sulphamoylbiphenyl-4-yl)-1-oxopropan-2-yl]cyclohexanecarboxamide hydrochloride

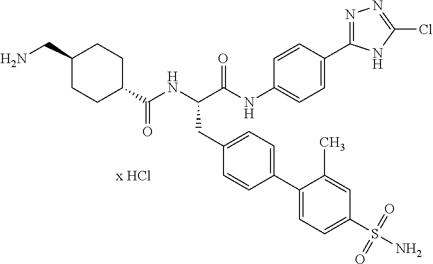 [SMILES: Cc1cc(S(N)(=O)=O)ccc1-c1ccc(C[C@H](NC(=O)[C@H]2CC[C@H](CN)CC2)C(=O)Nc2ccc(-c3nnc(Cl)[nH]3)cc2)cc1]

36.4 μl (0.15 mmol) of 4M hydrogen chloride in dioxane were added to a solution of 15.6 mg (21 μmol) of tert-butyl [(trans-4-{[(2S)-1-{[4-(3-chloro-4H-1,2,4-triazol-5-yl)phenyl]amino}-3-(2′-methyl-4′-sulphamoylbiphenyl-4-yl)-1-oxopropan-2-yl]carbamoyl}cyclohexyl)methyl]carbamate in 0.6 ml of dioxane. The mixture was stirred at RT for 3 days. The mixture was stirred at 40° C. for 2 h, 3.5 eq. of 4M hydrogen chloride in dioxane were then added and the mixture was then stirred overnight again at 30° C. Conversion incomplete. Another 1 eq. of 4M hydrogen chloride in dioxane was added, and the mixture was stirred at 30° C. overnight and then at 50° C. for another 2 h. Acetonitrile was added to the suspension and the precipitated solid was filtered off with suction and washed with acetonitrile. The solid was then dried under high vacuum and the residue was purified by preparative HPLC (Method 8). This gave 2.5 mg (16% of theory) of the title compound.

LC-MS (Method 4): Rₜ=0.83 min; MS (ESIpos): m/z=650 [M+H—HCl]⁺

¹H NMR (400 MHz, DMSO-d₆) δ ppm 0.76-0.97 (m, 2H), 1.09-1.26 (m, 2H), 1.47-1.58 (m, 1H), 1.63-1.79 (m, 3H), 2.05-2.17 (m, 1H), 2.21 (s, 3H), 2.27-2.31 (m, 1H), 2.60-2.65 (m, 1H), 2.84-2.95 (m, 1H), 3.09 (dd, 2H), 4.38-4.82 (m, 1H), 7.22 (d, 2H), 7.31 (d, 1H), 7.37 (d, 2H), 7.60 (d, 2H), 7.65 (dd, 1H), 7.70 (s, 1H), 7.82 (d, 2H), 8.17 (d, 1H), 8.35 (s, 1H), 10.19 (s, 1H).

### Example 21

### trans-4-(Aminomethyl)-N-[(2S)-3-[4′-(methylsulphonyl)-2′-(trifluoromethyl)biphenyl-4-yl]-1-oxo-1-{[4-(2H-tetrazol-5-yl)phenyl]amino}propan-2-yl]cyclohexanecarboxamide hydrochloride

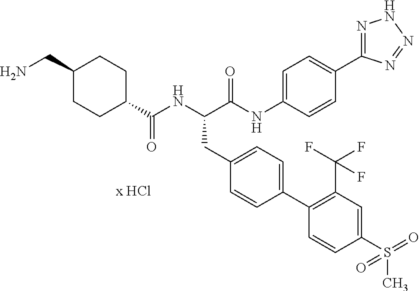 [SMILES: CS(=O)(=O)c1ccc(-c2ccc(C[C@H](NC(=O)[C@H]3CC[C@H](CN)CC3)C(=O)Nc3ccc(-c4nn[nH]n4)cc3)cc2)c(C(F)(F)F)c1]

0.09 ml (0.37 mmol) of 4M hydrogen chloride in dioxane were added to a solution of 19 mg (0.025 mmol) of tert-butyl [(trans-4-{[(2S)-3-[4′-(methylsulphonyl)-2′-(trifluoromethyl)biphenyl-4-yl]-1-oxo-1-{[4-(2H-tetrazol-5-yl)phenyl]amino}propan-2-yl]carbamoyl}cyclohexyl)methyl]carbamate in 1.5 ml of dioxane. The mixture was stirred at RT for 16 h. The precipitated product was filtered off with suction, washed with dioxane and dried under high vacuum. 11 mg (55% of theory, 91% purity) of the title compound were obtained.

LC-MS (Method 1): Rₜ=0.75 min; MS (ESIpos): m/z=670 [M+H—HCl]⁺

¹H NMR (400 MHz, DMSO-d₆) δ ppm=0.82-1.00 (m, 2H), 1.10-1.33 (m, 3H), 1.39-1.50 (m, 1H), 1.52-1.63 (m, 1H), 1.66-1.81 (m, 4H), 2.07-2.19 (m, 1H), 2.61-2.69 (m, 3H), 2.91-3.01 (m, 1H), 3.39 (s, 3H), 4.74-4.82 (m, 1H), 7.30 (d, 2H), 7.42 (d, 2H), 7.64 (d, 1H), 7.68-7.74 (m, 2H), 7.84 (d, 2H), 8.01 (d, 2H), 8.22-8.32 (m, 3H), 10.54 (s, 1H).

### Example 22

### trans-4-(Aminomethyl)-N-[(2S)-1-{[4-(3-chloro-4H-1,2,4-triazol-5-yl)phenyl]amino}-1-oxo-3-(4′-sulphamoylbiphenyl-4-yl)propan-2-yl]cyclohexanecarboxamide hydrochloride

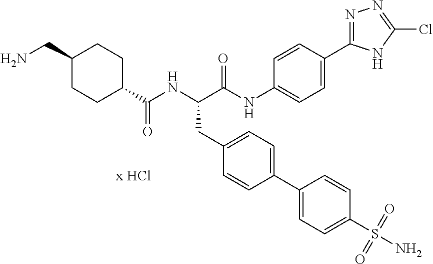 [SMILES: NC[C@H]1CC[C@H](C(=O)N[C@@H](Cc2ccc(-c3ccc(S(N)(=O)=O)cc3)cc2)C(=O)Nc2ccc(-c3nnc(Cl)[nH]3)cc2)CC1]

60.6 μl (0.15 mmol) of 4M hydrogen chloride in dioxane were added to a solution of 25.5 mg (21 μmol) of tert-butyl [(trans-4-{[(2S)-1-{[4-(3-chloro-4H-1,2,4-triazol-5-yl)phenyl]amino}-1-oxo-3-(4′-sulphamoylbiphenyl-4-yl)propan-2-yl]carbamoyl}cyclohexyl)methyl]carbamate in 0.9 ml of dioxane. The mixture was stirred at RT overnight. Since the conversion was incomplete, the mixture was stirred at 40° C. for 2 h and at 30° C. overnight. Acetonitrile was added to the suspension, and the precipitated solid was filtered off with suction and washed with acetonitrile. The solid was then dried under high vacuum and the residue was purified by preparative HPLC (Method 8). 17 mg (23% of theory, 93% purity) of the title compound were obtained.

LC-MS (Method 4): Rₜ=0.79 min; MS (ESIpos): m/z=636 [M+H—HCl]⁺

¹H NMR (300 MHz, DMSO-d₆) δ ppm 0.67-0.98 (m, 2H), 1.04-1.32 (m, 2H), 1.33-1.49 (m, 1H), 1.53-1.62 (m, 1H), 1.63-1.79 (m, 3H), 2.05-2.18 (m, 1H), 2.60 (t, 2H), 2.85-2.98 (m, 1H), 3.09 (dd, 1H), 4.56-4.79 (m, 1H), 7.34 (s, 1H), 7.42 (d, 2H), 7.62 (s, 2H), 7.74 (d, 5H), 7.82 (d, 2H), 7.87 (s, 2H), 8.18 (d, 1H), 10.07-10.61 (m, 1H).

### Example 23

### trans-4-(Aminomethyl)-N-[(2S)-3-(4′-cyano-2′-methylbiphenyl-4-yl)-1-oxo-1-{[4-(2H-tetrazol-5-yl)phenyl]amino}propan-2-yl]cyclohexanecarboxamide hydrochloride

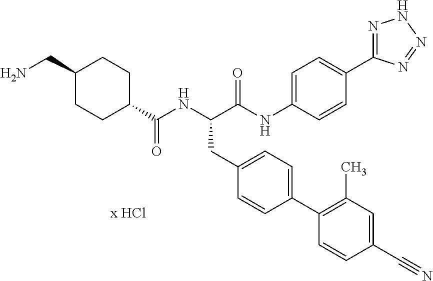 [SMILES: Cc1cc(C#N)ccc1-c1ccc(C[C@H](NC(=O)[C@H]2CC[C@H](CN)CC2)C(=O)Nc2ccc(-c3nn[nH]n3)cc2)cc1]

67.9 μl (2.72 mmol) of 4M hydrogen chloride in dioxane were added to a solution of 60 mg (91 μmol) of tert-butyl [(trans-4-{[(2S)-3-(4′-cyano-2′-methylbiphenyl-4-yl)-1-oxo-1-{[4-(2H-tetrazol-5-yl)phenyl]amino}propan-2-yl]carbamoyl}cyclohexyl)methyl]carbamate in 6 ml of tetrahydrofuran. The mixture was stirred at RT for 16 h. The precipitated product was filtered off with suction, washed with tetrahydrofuran and dried under high vacuum. This gave 41 mg (57% of theory) of the title compound.

LC-MS (Method 1): Rₜ=0.78 min; MS (ESIpos): m/z=563 [M+H—HCl]⁺

¹H NMR (400 MHz, DMSO-d₆) δ ppm 0.84-0.99 (m, 2H), 1.10-1.33 (m, 2H), 1.41-1.50 (m, 1H), 1.55 (br. s., 1H), 1.67-1.81 (m, 4H), 2.15 (br. s., 1H), 2.22 (s, 3H), 2.63 (t, 2H), 2.90-3.02 (m, 1H), 3.08-3.19 (m, 1H), 4.46-4.84 (m, 1H), 7.29 (d, 2H), 7.35 (d, 1H), 7.42 (d, 2H), 7.71 (d, 1H), 7.79 (br. s., 3H), 7.83 (d, 2H), 8.02 (d, 2H), 8.29 (d, 1H), 10.55 (br. s., 1H).

### Example 24

### trans-4-(Aminomethyl)-N-[(2S)-3-[4′-(dimethylsulphamoyl)-2′-methylbiphenyl-4-yl]-1-{[3-fluoro-4-(2H-tetrazol-5-yl)phenyl]amino}-1-oxopropan-2-yl]cyclohexanecarboxamide hydrochloride

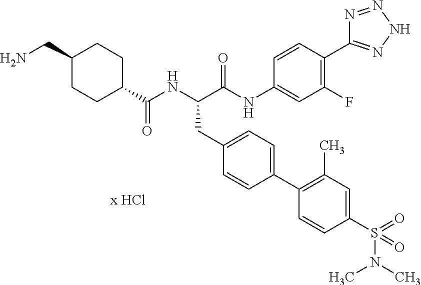 [SMILES: Cc1cc(S(=O)(=O)N(C)C)ccc1-c1ccc(C[C@H](NC(=O)[C@H]2CC[C@H](CN)CC2)C(=O)Nc2ccc(-c3nn[nH]n3)c(F)c2)cc1]

100 μl (0.40 mmol) of 4M hydrogen chloride in dioxane were added to a solution of 61.0 mg (80.0 μmol) of tert-butyl [(trans-4-{[(2S)-3-[4′-(dimethylsulphamoyl)-2′-methylbiphenyl-4-yl]-1-{[3-fluoro-4-(2H-tetrazol-5-yl)phenyl]amino}-1-oxopropan-2-yl]-carbamoyl}cyclohexyl)methyl]carbamate in 2 ml of dichloromethane. The mixture was stirred at RT overnight. Acetonitrile was added to the suspension, and the precipitated solid was filtered off with suction and washed with acetonitrile. The product was then dried under high vacuum. This gave 40 mg (70% of theory) of the title compound.

LC-MS (Method 4): Rₜ=0.91 min; MS (ESIpos): m/z=663 [M+H—HCl]⁺

¹H NMR (300 MHz, DMSO-d₆) δ ppm 0.82-1.00 (m, 2H), 1.09-1.32 (m, 2H), 1.53-1.62 (m, 1H), 1.67-1.81 (m, 3H), 2.07-2.19 (m, 1H), 2.26 (s, 3H), 2.64 (s, 7H), 2.90-3.03 (m, 1H), 3.09-3.18 (m, 1H), 4.67-4.78 (m, 1H), 7.32 (d, 2H), 7.37-7.44 (m, 3H), 7.52 (dd, 1H), 7.57-7.66 (m, 2H), 7.69-7.76 (m, 2H), 7.85 (dd, 1H), 8.02 (t, 1H), 8.30 (d, 1H), 10.58-10.83 (m, 1H).

### Example 25

### trans-4-(Aminomethyl)-N-[(2S)-1-oxo-3-[4′-(3-oxomorpholin-4-yl)biphenyl-4-yl]-1-{[4-(2H-tetrazol-5-yl)phenyl]amino}propan-2-yl]cyclohexanecarboxamide hydrochloride

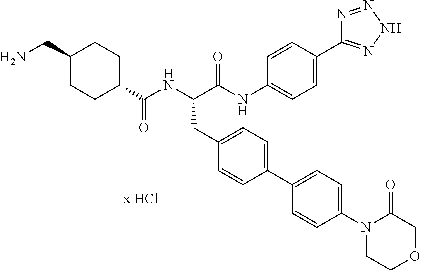 [SMILES: NC[C@H]1CC[C@H](C(=O)N[C@@H](Cc2ccc(-c3ccc(N4CCOCC4=O)cc3)cc2)C(=O)Nc2ccc(-c3nn[nH]n3)cc2)CC1]

74 μl (0.40 mmol) of 4M hydrogen chloride in dioxane were added to a solution of 21.4 mg (30.0 μmol) of tert-butyl [(trans-4-{[(2S)-1-oxo-3-[4′-(3-oxomorpholin-4-yl)biphenyl-4-yl]-1-{[4-(2H-tetrazol-5-yl)phenyl]amino}propan-2-yl]carbamoyl}cyclohexyl)methyl]carbamate in 1.3 ml of dioxane. The reaction mixture was stirred at 40° C. for 4 h and then overnight at RT. The suspension was purified by preparative HPLC (Method 7). 2.7 mg (13% of theory, 93% purity) of the title compound were obtained.

LC-MS (Method 4): Rₜ=0.76 min; MS (ESIpos): m/z=623 [M+H—HCl]⁺

¹H NMR (300 MHz, DMSO-d₆) δ ppm 0.77-0.99 (m, 2H), 1.08-1.30 (m, 2H), 1.36-1.47 (m, 1H), 1.51-1.62 (m, 1H), 1.63-1.82 (m, 3H), 2.06-2.17 (m, 1H), 2.55-2.64 (m, 2H), 2.83-2.97 (m, 1H), 3.02-3.13 (m, 1H), 3.67-3.78 (m, 2H), 3.91-4.01 (m, 2H), 4.18 (s, 2H), 4.64-4.75 (m, 1H), 7.36 (d, 2H), 7.44 (d, 2H), 7.57 (d, 2H), 7.60-7.71 (m, 4H), 7.79 (d, 2H), 7.96 (d, 2H), 8.17 (d, 1H), 10.46 (s, 1H).

### Example 26

### trans-N-[(2S)-3-(4′-Acetamido-3′-fluorobiphenyl-4-yl)-1-oxo-1-{[4-(2H-tetrazol-5-yl)phenyl]-amino}propan-2-yl]-4-(aminomethyl)cyclohexanecarboxamide hydrochloride

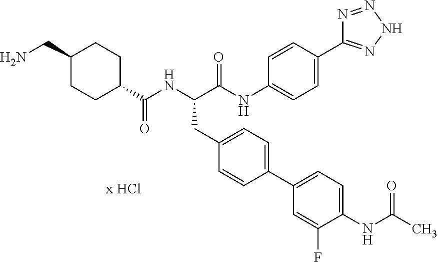 [SMILES: CC(=O)Nc1ccc(-c2ccc(C[C@H](NC(=O)[C@H]3CC[C@H](CN)CC3)C(=O)Nc3ccc(-c4nn[nH]n4)cc3)cc2)cc1F]

261 μl (1.04 mmol) of 4M hydrogen chloride in dioxane were added to a solution of 73.0 mg (104.5 μmol) of tert-butyl [(trans-4-{[(2S)-3-(4′-acetamido-3′-fluorobiphenyl-4-yl)-1-oxo-1-{[4-(2H-tetrazol-5-yl)phenyl]amino}propan-2-yl]carbamoyl}cyclohexyl)methyl]carbamate in 6.3 ml of dichloromethane. The mixture was stirred at RT overnight. Acetonitrile was added to the suspension, the resulting precipitate was filtered off with suction and the solid was purified by preparative HPLC (Method 7). This gave 22 mg (33% of theory) of the title compound.

LC-MS (Method 4): Rₜ=0.75 min; MS (ESIpos): m/z=599 [M+H—HCl]⁺

¹H NMR (400 MHz, DMSO-d₆) δ ppm 0.79-1.02 (m, 2H), 1.11-1.33 (m, 2H), 1.43 (br. s, 1H), 1.59 (m, 1H), 1.66-1.81 (m, 3H), 2.06-2.18 (m, 4H), 2.63 (d, 2H), 2.89 (dd, 1H), 3.10 (dd, 1H), 4.58-4.77 (m, 1H), 7.38 (d, 2H), 7.46 (dd, 1H), 7.55 (dd, 1H), 7.60 (m, 4H), 7.89 (d, 2H), 7.94 (t, 1H), 8.11 (d, 1H), 9.75 (s, 1H), 10.12 (s, 1H).

### Example 27

### trans-4-(Aminomethyl)-N-[(2S)-3-[4′-methoxy-2′-(trifluoromethyl)biphenyl-4-yl]-1-oxo-1-{[4-(2H-tetrazol-5-yl)phenyl]amino}propan-2-yl]cyclohexanecarboxamide hydrochloride

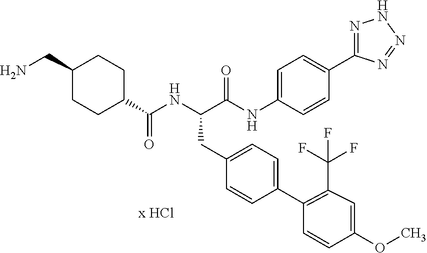

0.23 ml (0.91 mmol) of 4M hydrogen chloride in dioxane were added to a solution of 76 mg (91 μmol) of tert-butyl [(trans-4-{[(2S)-3-[4′-methoxy-2′-(trifluoromethyl)biphenyl-4-yl]-1-oxo-1-{[4-(2H-tetrazol-5-yl)phenyl]amino}propan-2-yl]carbamoyl}cyclohexyl)methyl]carbamate in 2 ml of dioxane. The mixture was stirred at RT and after 4 h a further 10 eq. of 4M hydrogen chloride in dioxane were added. After 16 h, the precipitated product was filtered off with suction, taken up in a little methanol and purified by preparative HPLC (mobile phase: gradient of acetonitrile/water with 0.1% trifluoroacetic acid). The product-containing fractions were combined, 0.2 ml of 4M hydrogen chloride in 1,4-dioxane was added and the mixture was concentrated on a rotary evaporator. The residue was dried under high vacuum. 49 mg (76% of theory, 94% purity) of the title compound were obtained.

LC-MS (Method 1): Rₜ=0.84 min; MS (ESIpos): m/z=622 [M+H—HCl]⁺.

¹H NMR (400 MHz, DMSO-d₆) δ ppm 0.83-1.01 (m, 2H), 1.10-1.35 (m, 2H), 1.41-1.51 (m, 1H), 1.53-1.59 (m, 1H), 1.70-1.82 (m, 3H), 2.10-2.20 (m, 1H), 2.63 (t, 2H), 2.95 (t, 1H), 3.09-3.17 (m, 1H), 3.87 (s, 3H), 4.69-4.80 (m, 1H), 7.20 (d, 2H), 7.24-7.30 (m, 3H), 7.36 (d, 2H), 7.69-7.79 (m, 3H), 7.83 (d, 2H), 8.01 (d, 2H), 8.24 (d, 1H), 10.53 (s, 1H).

### Example 28

### trans-4-(Aminomethyl)-N-{(2S)-3-[4′-{[4-(dimethylamino)cyclohexyl]sulphamoyl}-2′-(trifluoromethyl)biphenyl-4-yl]-1-oxo-1-[(2-oxo-2,3-dihydro-1H-benzimidazol-5-yl)amino]propan-2-yl}cyclohexanecarboxamide hydrochloride

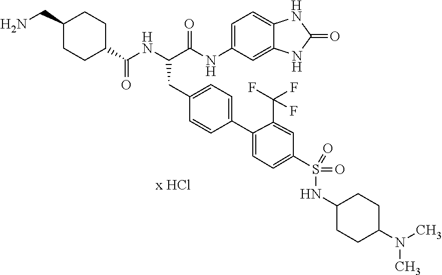

0.04 ml (0.15 mmol) of 4M hydrogen chloride in 1,4-dioxane was added to a solution of 34 mg (0.04 mmol) of tert-butyl {[trans-4-({(2S)-3-[4′-{[4-(dimethylamino)cyclohexyl]sulphamoyl}-2′-(trifluoromethyl)biphenyl-4-yl]-1-oxo-1-[(2-oxo-2,3-dihydro-1H-benzimidazol-5-yl)amino]propan-2-yl}carbamoyl)cyclohexyl]methyl}carbamate in 2 ml of dichloromethane, and the mixture was stirred at RT overnight. After addition of a further 0.02 ml (0.08 mmol) of 4M hydrogen chloride in 1,4-dioxane, acetonitrile was added to the reaction mixture and the resulting solid was filtered off, washed with acetonitrile and dried under high vacuum. This gave 21 mg (62% of theory) of the title compound.

¹H NMR (300 MHz, DMSO-d₆) δ ppm 0.76-1.00 (m, 2H), 1.07-1.34 (m, 5H), 1.36-1.59 (m, 4H), 1.65-1.83 (m, 5H), 1.86-1.99 (m, 2H), 2.06-2.19 (m, 1H), 2.61 (br. s., 6H), 2.87-2.98 (m, 1H), 2.99-3.17 (m, 4H), 4.58-4.82 (m, 1H), 6.77-6.88 (m, 1H), 6.97-7.08 (m, 1H), 7.19-7.30 (m, 2H), 7.35-7.47 (m, 3H), 7.54-7.63 (m, 1H), 7.70-7.90 (m, 3H), 8.03-8.14 (m, 2H), 8.14-8.24 (m, 2H), 9.97-10.08 (m, 1H), 10.09-10.25 (m, 1H), 10.45-10.54 (m, 1H), 10.54-10.62 (m, 1H).

LC-MS (Method 4): Rₜ=0.68 min; MS (ESIneg): m/z=782.8 [M−H]⁻.

### Example 29

### trans-4-(Aminomethyl)-N-{(2S)-3-[4′-(cyclopropylsulphamoyl)-2′-methylbiphenyl-4-yl]-1-oxo-1-[(3-oxo-2,3-dihydro-1H-indazol-6-yl)amino]propan-2-yl}cyclohexanecarboxamide hydrochloride

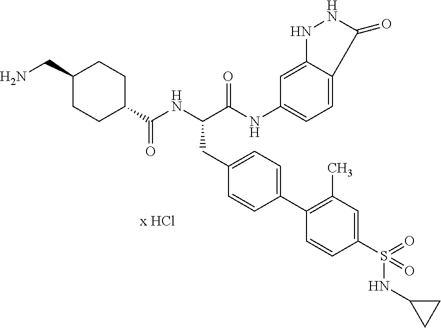 [SMILES: Cc1cc(S(=O)(=O)NC2CC2)ccc1-c1ccc(C[C@H](NC(=O)[C@H]2CC[C@H](CN)CC2)C(=O)Nc2ccc3c(=O)[nH][nH]c3c2)cc1]

4M hydrogen chloride in dioxane is added to a solution of tert-butyl {[trans-4-({(2S)-3-[4′-(cyclopropylsulphamoyl)-2′-methylbiphenyl-4-yl]-1-oxo-1-[(3-oxo-2,3-dihydro-1H-indazol-6-yl)amino]propan-2-yl}carbamoyl)cyclohexyl]methyl}carbamate in dichloromethane. The mixture is stirred at RT overnight. The reaction mixture is worked up by methods known to those skilled in the art and the residue is separated by means of preparative HPLC. This gives the title compound.

### Example 30

### trans-4-(Aminomethyl)-N-{(2S)-3-[4′-(dimethylsulphamoyl)-2′-methylbiphenyl-4-yl]-1-oxo-1-[(3-oxo-2,3-dihydro-1H-indazol-6-yl)amino]propan-2-yl}cyclohexanecarboxamide hydrochloride

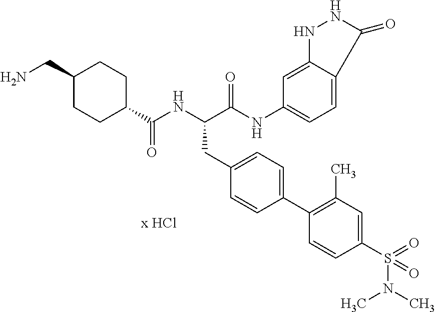 [SMILES: Cc1cc(S(=O)(=O)N(C)C)ccc1-c1ccc(C[C@H](NC(=O)[C@H]2CC[C@H](CN)CC2)C(=O)Nc2ccc3c(=O)[nH][nH]c3c2)cc1]

4M hydrogen chloride in dioxane is added to a solution of tert-butyl {[trans-4-({(2S)-3-[4′-(dimethylsulphamoyl)-2′-methylbiphenyl-4-yl]-1-oxo-1-[(3-oxo-2,3-dihydro-1H-indazol-6-yl)amino]propan-2-yl}carbamoyl)cyclohexyl]methyl}carbamate in dichloromethane. The mixture is stirred at RT overnight. The reaction mixture is worked up by methods known to those skilled in the art and the residue is separated by means of preparative HPLC. This gives the title compound.

### Example 31

### trans-4-(Aminomethyl)-N-{(2S)-3-[4′-(isopropylsulphamoyl)-2′-methylbiphenyl-4-yl]-1-oxo-1-[(3-oxo-2,3-dihydro-1H-indazol-6-yl)amino]propan-2-yl}cyclohexanecarboxamide hydrochloride

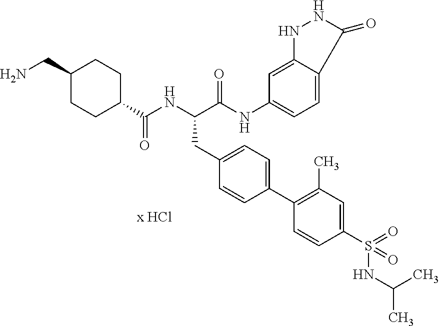 [SMILES: Cc1cc(S(=O)(=O)NC(C)C)ccc1-c1ccc(C[C@H](NC(=O)[C@H]2CC[C@H](CN)CC2)C(=O)Nc2ccc3c(=O)[nH][nH]c3c2)cc1]

4M hydrogen chloride in dioxane is added to a solution of tert-butyl {[trans-4-({(2S)-3-[4′-(isopropylsulphamoyl)-2′-methylbiphenyl-4-yl]-1-oxo-1-[(3-oxo-2,3-dihydro-1H-indazol-6-yl)amino]propan-2-yl}carbamoyl)cyclohexyl]methyl}carbamate in dichloromethane. The mixture is stirred at RT overnight. The reaction mixture is worked up by methods known to those skilled in the art and the residue is separated by means of preparative HPLC. This gives the title compound.

### Example 32

### trans-4-(Aminomethyl)-N-{(2S)-3-[2′-methyl-4′-(pyrrolidin-1-ylsulphonyl)biphenyl-4-yl]-1-oxo-1-[(3-oxo-2,3-dihydro-1H-indazol-6-yl)amino]propan-2-yl}cyclohexanecarboxamide hydrochloride

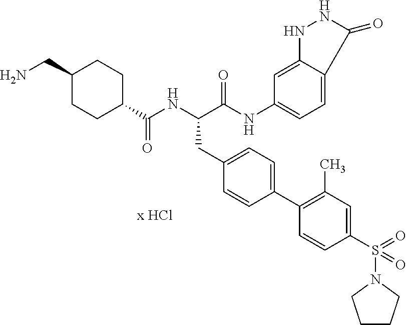 [SMILES: Cc1cc(S(=O)(=O)N2CCCC2)ccc1-c1ccc(C[C@H](NC(=O)[C@H]2CC[C@H](CN)CC2)C(=O)Nc2ccc3c(=O)[nH][nH]c3c2)cc1]

4M hydrogen chloride in dioxane is added to a solution of tert-butyl {[trans-4-({(2S)-3-[2′-methyl-4′-(pyrrolidin-1-ylsulphonyl)biphenyl-4-yl]-1-oxo-1-[(3-oxo-2,3-dihydro-1H-indazol-6-yl)amino]propan-2-yl}carbamoyl)cyclohexyl]methyl}carbamate in dichloromethane. The mixture is stirred at RT overnight. The reaction mixture is worked up by methods known to those skilled in the art and the residue is separated by means of preparative HPLC. This gives the title compound.

### Example 33

### trans-4-(Aminomethyl)-N-{(2S)-3-[4′-(diethylsulphamoyl)-2′-methylbiphenyl-4-yl]-1-oxo-1-[(3-oxo-2,3-dihydro-1H-indazol-6-yl)amino]propan-2-yl}cyclohexanecarboxamide hydrochloride

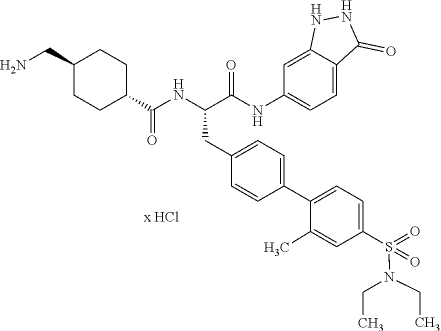 [SMILES: CCN(CC)S(=O)(=O)c1ccc(-c2ccc(C[C@H](NC(=O)[C@H]3CC[C@H](CN)CC3)C(=O)Nc3ccc4c(=O)[nH][nH]c4c3)cc2)c(C)c1]

4M hydrogen chloride in dioxane is added to a solution of tert-butyl {[trans-4-({(2S)-3-[4′-(diethylsulphamoyl)-2′-methylbiphenyl-4-yl]-1-oxo-1-[(3-oxo-2,3-dihydro-1H-indazol-6-yl)amino]propan-2-yl}carbamoyl)cyclohexyl]methyl}carbamate in dichloromethane. The mixture is stirred at RT overnight. The reaction mixture is worked up by methods known to those skilled in the art and the residue is separated by means of preparative HPLC. This gives the title compound.

### Example 34

### trans-4-(Aminomethyl)-N-{(2S)-3-[2′-methyl-4′-(methylsulphamoyl)biphenyl-4-yl]-1-oxo-1-[(3-oxo-2,3-dihydro-1H-indazol-6-yl)amino]propan-2-yl}cyclohexanecarboxamide hydrochloride

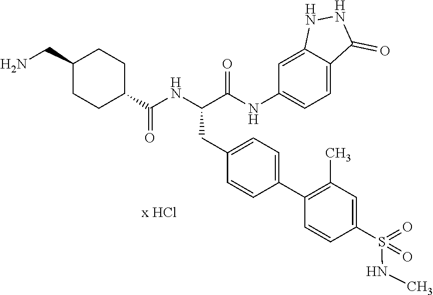 [SMILES: CNS(=O)(=O)c1ccc(-c2ccc(C[C@H](NC(=O)[C@H]3CC[C@H](CN)CC3)C(=O)Nc3ccc4c(=O)[nH][nH]c4c3)cc2)c(C)c1]

4M hydrogen chloride in dioxane is added to a solution of tert-butyl {[trans-4-({(2S)-3-[2′-methyl-4′-(methylsulphamoyl)biphenyl-4-yl]-1-oxo-1-[(3-oxo-2,3-dihydro-1H-indazol-6-yl)amino]propan-2-yl}carbamoyl)cyclohexyl]methyl}carbamate in dichloromethane. The mixture is stirred at RT overnight. The reaction mixture is worked up by methods known to those skilled in the art and the residue is separated by means of preparative HPLC. This gives the title compound.

### B) ASSESSMENT OF PHYSIOLOGICAL EFFICACY

The suitability of the compounds according to the invention for treating thromboembolic or hyperfibrinolytic disorders can be demonstrated in the following assay systems:

a) Test Descriptions (In Vitro)

a.1) Measurement of FXIa Inhibition

The factor XIa inhibition of the substances according to the invention is determined using a biochemical test system which utilizes the reaction of a peptidic factor XIa substrate to determine the enzymatic activity of human factor XIa. Here, factor XIa cleaves from the peptic factor XIa substrate the C-terminal aminomethylcoumarin (AMC), the fluorescence of which is measured. The determinations are carried out in microtitre plates.

Test substances are dissolved in dimethyl sulphoxide and serially diluted in dimethyl sulphoxide (3000 μM to 0.0078 μM; resulting final concentrations in the test: 50 μM to 0.00013 μM). In each case 1 μl of the diluted substance solutions is placed into the wells of white microtitre plates from Greiner (384 wells). Subsequently, the following are added successively: 20 μl of assay buffer (50 mmol/l Tris buffer pH 7.4; 100 mmol/l sodium chloride; 5 mmol/l calcium chloride; 0.1% bovine serum albumin) and 20 μl of factor XIa from Kordia (0.45 nM in assay buffer). After 15 min of incubation, the enzyme reaction is started by addition of 20 μl of the factor XIa substrate Boc-Glu(OBzl)-Ala-Arg-AMC dissolved in assay buffer (10 μM in assay buffer) from Bachem, the mixture is incubated at room temperature (22° C.) for 30 min and fluorescence is then measured (excitation: 360 nm, emission: 460 nm). The measured emissions of the test batches with test substance are compared to those of control batches without test substance (only dimethyl sulphoxide instead of test substance in dimethyl sulphoxide), and IC₅₀ values are calculated from the concentration/activity relationships. Activity data from this test are listed in Table A below:

TABLE A

| Example No. | IC₅₀ [nM] | Example No. | IC₅₀ [nM] |
|---|---|---|---|
| 1 | 1.1 | 2 | 4.3 |
| 3 | 8.1 | 4 | 18 |
| 5 | 5.7 | 6 | 24 |
| 7 | 3.6 | 8 | 1.0 |
| 9 | 8.4 | 10 | 1.1 |
| 11 | 1.9 | 12 | 2.7 |
| 13 | 3.1 | 14 | 3.2 |
| 15 | 2.8 | 16 | 1.3 |
| 17 | 3.5 | 18 | 5.4 |
| 19 | 2.3 | 20 | 1.8 |
| 21 | 0.9 | 22 | 1.5 |
| 23 | 1.1 | 24 | 0.4 |
| 25 | 2.5 | 26 | 4.7 |
| 27 | 4.5 | 28 | 5.2 |

a.2) Determination of the Selectivity

To demonstrate the selectivity of the substances with respect to FXIa inhibition, the test substances are examined for their inhibition of other human serine proteases, such as factor Xa, trypsin and plasmin. To determine the enzymatic activity of factor Xa (1.3 nmol/l from Kordia), trypsin (83 mU/ml from Sigma) and plasmin (0.1 μg/ml from Kordia), these enzymes are dissolved (50 mmol/l of Tris buffer [C,C,C-tris(hydroxymethyl)aminomethane], 100 mmol/l of sodium chloride, 0.1% BSA [bovine serum albumin], 5 mmol/l of calcium chloride, pH 7.4) and incubated for 15 min with test substance in various concentrations in dimethyl sulphoxide and also with dimethyl sulphoxide without test substance. The enzymatic reaction is then started by addition of the appropriate substrates (5 μmol/l of Boc-Ile-Glu-Gly-Arg-AMC from Bachem for factor Xa and trypsin, 50 μmol/l of MeOSuc-Ala-Phe-Lys-AMC from Bachem for plasmin). After an incubation time of 30 min at 22° C., fluorescence is measured (excitation: 360 nm, emission: 460 nm). The measured emissions of the test mixtures with test substance are compared to the control mixtures without test substance (only dimethyl sulphoxide instead of test substance in dimethyl sulphoxide) and IC₅₀ values are calculated from the concentration/activity relationships.

a.3) Thrombin Generation Assay (Thrombogram)

The effect of the test substances on the thrombogram (thrombin generation assay according to Hemker) is determined in vitro in human plasma (Octaplas® from Octapharma).

In the thrombin generation assay according to Hemker, the activity of thrombin in coagulating plasma is determined by measuring the fluorescent cleavage products of the substrate I-1140 (Z-Gly-Gly-Arg-AMC, Bachem). The reactions are carried out in the presence of varying concentrations of test substance or the corresponding solvent. To start the reaction, reagents from Thrombinoscope (30 pM or 0.1 pM recombinant tissue factor, 24 μM phospholipids in HEPES) are used. In addition, a thrombin calibrator from Thrombinoscope is used whose amidolytic activity is required for calculating the thrombin activity in a sample containing an unknown amount of thrombin. The test is carried out according to the manufacturer's instructions (Thrombinoscope BV): 4 μl of test substance or of the solvent, 76 μl of plasma and 20 μl of PPP reagent or thrombin calibrator are incubated at 37° C. for 5 min After addition of 20 μl of 2.5 mM thrombin substrate in 20 mM HEPES, 60 mg/ml of BSA, 102 mM of calcium chloride, the thrombin generation is measured every 20 s over a period of 120 min. Measurement is carried out using a fluorometer (Fluoroskan Ascent) from Thermo Electron fitted with a 390/460 nm filter pair and a dispenser.

Using the Thrombinoscope software, the thrombogram is calculated and represented graphically. The following parameters are calculated: lag time, time to peak, peak, ETP (endogenous thrombin potential) and start tail.

a.4) Determination of Anticoagulatory Activity

The anticoagulatory activity of the test substances is determined in vitro in human and animal plasma (for example mouse, rat, rabbit, pig and dog plasma). To this end, blood is drawn off in a mixing ratio of sodium citrate/blood of 1:9 using a 0.11 molar sodium citrate solution as receiver. Immediately after the blood has been drawn off, it is mixed thoroughly and centrifuged at about 4000 g for 15 minutes. The supernatant is pipetted off.

The prothrombin time (PT, synonyms: thromboplastin time, quick test) is determined in the presence of varying concentrations of test substance or the corresponding solvent using a commercial test kit (Neoplastin® from Boehringer Mannheim or Hemoliance® RecombiPlastin from Instrumentation Laboratory). The test compounds are incubated with the plasma at 37° C. for 3 minutes. Coagulation is then started by addition of thromboplastin, and the time when coagulation occurs is determined The concentration of test substance which effects a doubling of the prothrombin time is determined

The activated partial thromboplastin time (aPTT) is determined in the presence of varying concentrations of test substance or the corresponding solvent using a commercial test kit (C.K. Prest from Diagnostica Stago). The test compounds are incubated with the plasma and the PTT reagent (cephalin, kaolin) at 37° C. for 3 minutes. Coagulation is then started by addition of a 25 mM aqueous calcium chloride solution, and the time when coagulation occurs is determined. The concentration of test substance which brings about a 1.5-fold extension of the aPTT is determined. Activity data from this test are listed in Table B below:

TABLE B

| Example No. | aPTT [μmol/l] | Example No. | aPTT [μmol/l] |
|---|---|---|---|
| 1 | 0.1 | 2 | 0.26 |
| 3 | 0.29 | 4 | 0.3 |
| 5 | 0.39 | 6 | 0.15 |
| 7 | 0.37 | 8 | 0.08 |
| 9 | 0.22 | 10 | 0.1 |
| 11 | 0.49 | 12 | 0.04 |
| 13 | 0.07 | 14 | 0.09 |
| 15 | 0.09 | 16 | 0.11 |
| 17 | 0.13 | 18 | 0.13 |
| 19 | 0.16 | 20 | 0.18 |
| 21 | 0.23 | 22 | 0.25 |
| 23 | 0.27 | 24 | 0.27 |
| 25 | 0.36 | 26 | 0.4 |
| 27 | 0.21 | 28 | 0.06 |

a.5) Determination of Fibrinolytic Activity

Antifibrinolytic activity in vitro is assessed in human, platelet-free plasma. Tissue factor (TF) (1 pM) and tissue plasminogen activator (tPA) (40 nM) are pipetted into plasma together with 12.5 mM aqueous calcium chloride solution and substance. On occurrence of clotting, the subsequent clot lysis is determined photometrically over a period of 30 minutes.

a.6) Measurement of Plasmin Inhibition

The plasmin inhibition of the inventive substances is determined using a biochemical test system which utilizes the reaction of a peptidic plasmin substrate to determine the enzymatic activity of human plasmin. Here, plasmin cleaves from the peptic plasmin substrate the C-terminal aminomethylcoumarin (AMC), the fluorescence of which is measured. The determinations are carried out in microtitre plates.

Test substances are dissolved in dimethyl sulphoxide and serially diluted in dimethyl sulphoxide (3000 μM to 0.0078 μM; resulting final concentrations in the test: 50 μM to 0.00013 μM). In each case 1 μl of the diluted substance solutions is placed into the wells of white microtitre plates from Greiner (384 wells). Subsequently, the following are added successively: 20 μl of assay buffer (50 mmol/l Tris buffer pH 7.4; 100 mmol/l sodium chloride; 5 mmol/l calcium chloride; 0.1% bovine serum albumin) and 20 μl of plasmin from Kordia (0.3 μg/ml in assay buffer). After 15 min of incubation, the enzyme reaction is started by addition of 20 μl of the plasmin substrate MeOSuc-Ala-Phe-Lys-AMC dissolved in assay buffer (150 μM in assay buffer) from Bachem, the mixture is incubated at room temperature (22° C.) for 30 min and fluorescence is then measured (excitation: 360 nm, emission: 460 nm). The measured emissions of the test batches with test substance are compared to those of control batches without test substance (only dimethyl sulphoxide instead of test substance in dimethyl sulphoxide), and IC₅₀ values are calculated from the concentration/activity relationships. Activity data from this test are listed in Table C below:

TABLE C

| Example No. | IC₅₀ [nM] |
|---|---|
| 12 | 30 |
| 15 | 23 |

b) Determination of Antithrombotic Activity (In Vivo)

b.1) Arterial Thrombosis Model (Iron(II) Chloride-Induced Thrombosis) in Combination with Ear Bleeding Time in Rabbits

The antithrombotic activity of the FXIa inhibitors is tested in an arterial thrombosis model. Thrombus formation is triggered here by causing chemical injury to a region in the carotid artery in rabbits. Simultaneously, the ear bleeding time is determined

Male rabbits (Crl:KBL (NZW)BR, Charles River) receiving a normal diet and having a body weight of 2.2-2.5 kg are anaesthetized by intramuscular administration of xylazine and ketamine (Rompun, Bayer, 5 mg/kg and Ketavet, Pharmacia & Upjohn GmbH, 40 mg/kg body weight). Anaesthesia is furthermore maintained by intravenous administration of the same preparations (bolus: continuous infusion) via the right auricular vein.

The right carotid artery is exposed and the vessel injury is then caused by wrapping a piece of filter paper (10 mm×10 mm) on a Parafilm® strip (25 mm×12 mm) around the carotid artery without disturbing the blood flow. The filter paper contains 100 μL of a 13% strength solution of iron(II) chloride (Sigma) in water. After 5 min, the filter paper is removed and the vessel is rinsed twice with aqueous 0.9% strength sodium chloride solution. 30 min after the injury the injured region of the carotid artery is extracted surgically and any thrombotic material is removed and weighed.

The test substances are administered either intravenously to the anaesthetized animals via the femoral vein or orally to the awake animals via gavage, in each case 5 min and 2 h, respectively, before the injury.

Ear bleeding time is determined 2 min after injury to the carotid artery. To this end, the left ear is shaved and a defined 3-mm-long incision (blade Art. Number 10-150-10, Martin, Tuttlingen, Germany) is made parallel to the longitudinal axis of the ear. Care is taken here not to damage any visible vessels. Any blood that extravasates is taken up in 15 second intervals using accurately weighed filter paper pieces, without touching the wound directly. Bleeding time is calculated as the time from making the incision to the point in time where no more blood can be detected on the filter paper. The volume of the extravasated blood is calculated after weighing of the filter paper pieces.

c) Determination of Fibrinolytic Activity (In Vivo)

c.1) Hyper-Fibrinolytic Rats

The determination of antifibrinolytic activity in vivo is conducted in hyperfibrinolytic rats. After anaesthetization and catheterization of the animals, hyperfibrinolysis is triggered by infusion of tissue plasminogen activator (tPA) (8 mg/kg/h). 10 minutes after commencement of tPA infusion, the substances are administered as an i.v. bolus. After a further 15 minutes, tPA infusion is ended and a transsection of the tail is conducted. Subaqual bleeding (in physiological saline at 37° C.) is observed over 30 minutes and the bleed time is determined.

### C) WORKING EXAMPLES OF PHARMACEUTICAL PREPARATIONS

The substances according to the invention can, for example, be converted to pharmaceutical preparations as follows:

Tablet:

Composition:

100 mg of the compound of Example 1, 50 mg of lactose (monohydrate), 50 mg of maize starch, 10 mg of polyvinylpyrrolidone (PVP) and 2 mg of magnesium stearate.

Tablet weight 212 mg. Diameter 8 mm, radius of curvature 12 mm

Production:

The mixture of the compound of Example 1, lactose and starch is granulated with a 5% strength solution (m/m) of the PVP in water. After drying, the granules are mixed with the magnesium stearate for 5 min This mixture is compressed in a conventional tabletting press (see above for format of the tablet).

Oral Suspension:

Composition:

1000 mg of the compound of Example 1, 1000 mg of ethanol (96%), 400 mg of Rhodigel and 99 g of water.

10 ml of oral suspension correspond to a single dose of 100 mg of the compound of the invention.

Production:

The Rhodigel is suspended in ethanol, and the compound of Example 1 is added to the suspension. The water is added while stirring. The mixture is stirred for about 6 h until swelling of the Rhodigel is complete.

Solution for Oral Administration:

Composition:

500 mg of the compound of the invention, 2.5 g of polysorbate and 97 g of polyethylene glycol 400. 20 g of oral solution correspond to a single dose of 100 mg of the compound of the invention.

Production:

The compound of the invention is suspended in the mixture of polyethylene glycol and polysorbate with stirring. The stirring operation is continued until dissolution of the compound of the invention is complete.

I.V. Solution:

The compound of the invention is dissolved in a concentration below the saturation solubility in a physiologically acceptable solvent (e.g. isotonic saline solution, glucose solution 5% and/or polyethylene glycol 400/water 30% m/m). The solution is subjected to sterile filtration and dispensed into sterile and pyrogen-free injection vessels.

1. A compound of the formula 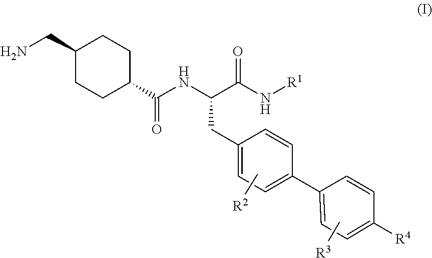
in which
R¹ represents a group of the formula 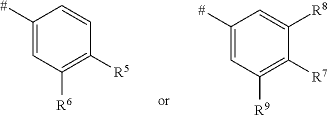
where # is the point of attachment to the nitrogen atom,
R⁵ represents 5-membered heteroaryl,
where heteroaryl may be substituted by a substituent selected from the group consisting of oxo, chlorine, cyano, hydroxy and C₁-C₃-alkyl,
in which alkyl may be substituted by 1 to 3 substituents independently of one another selected from the group consisting of hydroxy, amino, hydroxycarbonyl and methoxy, or
in which alkyl may be substituted by 1 to 7 fluorine substituents, or
in which alkyl is substituted by a substituent selected from the group consisting of hydroxy, amino, hydroxycarbonyl and methoxy, and in which alkyl is additionally substituted by 1 to 6 fluorine substituents,
R⁶ represents hydrogen, fluorine or chlorine,
R⁷ and R^{g} together with the carbon atoms to which they are attached form a 5-membered heterocycle,
where the heterocycle may be substituted by 1 to 2 substituents independently of one another selected from the group consisting of oxo, chlorine, cyano, hydroxyl, C₁-C₃-alkyl, pyrazolyl and pyridyl,
in which alkyl may be substituted by 1 to 3 substituents independently of one another selected from the group consisting of hydroxy, amino, hydroxycarbonyl and methoxy, or
in which alkyl may be substituted by 1 to 7 fluorine substituents, or
in which alkyl is substituted by a substituent selected from the group consisting of hydroxy, amino, hydroxycarbonyl and methoxy, and in which alkyl is additionally substituted by 1 to 6 fluorine substituents,
R⁹ represents hydrogen, fluorine or chlorine,
R² represents hydrogen, fluorine, chlorine, methyl or methoxy,
R³ represents hydrogen, fluorine, chlorine, C₁-C₄-alkyl, methoxy or trifluoromethyl,
R⁴ represents amino, cyano, C₁-C₃-alkoxy, C₁-C₃-alkylamino, C₁-C₃-alkoxycarbonyl, C₁-C₃-alkylcarbonylamino, C₁-C₃-alkylsulphonyl, —S(O)₂NR¹⁰R¹¹ or 5- to 7-membered heterocyclyl which is attached via a nitrogen atom,
where alkoxy may be substituted by 1 to 2 substituents independently of one another selected from the group consisting of fluorine, hydroxy, amino, hydroxycarbonyl, C₁-C₃-alkoxy, C₁-C₃-alkylamino, difluoromethyl, trifluoromethyl, —(OCH₂CH₂)ₙ—OCH₃, —(OCH₂CH₂)ₘ—OH, morpholinyl, piperidinyl and pyrrolidinyl,
in which n is a number from 1 to 6,
in which m is a number from 1 to 6, and
where alkylcarbonylamino may be substituted by a —NH(CO)CH₂NH(CO)CH₂NH₂ substituent, and
where heterocyclyl may be substituted by 1 to 2 substituents independently of one another selected from the group consisting of oxo, chlorine, cyano, hydroxy and C₁-C₄-alkyl, and
where
R¹⁰ represents hydrogen, C₁-C₃-alkyl, C₃-C₆-cycloalkyl, benzyl or 4- to 8-membered heterocyclyl which is attached via a carbon atom,
in which alkyl may be substituted by 1 to 2 substituents independently of one another selected from the group consisting of hydroxy, amino, hydroxycarbonyl and methoxy, and
in which cycloalkyl may be substituted by 1 to 2 substituents independently of one another selected from the group consisting of oxo, fluorine, hydroxy, amino, C₁-C₄-alkyl and C₁-C₃-alkylamino, and
in which heterocyclyl may be substituted by 1 to 2 substituents independently of one another selected from the group consisting of oxo, chlorine, cyano, hydroxy and C₁-C₄-alkyl,
R¹¹ represents hydrogen or C₁-C₃-alkyl, or
R¹⁰ and R¹¹ together with the nitrogen atom to which they are attached form a 4- to 7-membered heterocycle,
in which the heterocycle may be substituted by 1 to 2 substituents selected independently from the group consisting of oxo, fluorine, hydroxyl, amino, hydroxycarbonyl, C₁-C₄-alkyl, C₁-C₃-alkylamino, difluoromethyl, trifluoromethyl, 2,2,2-trifluoroeth-1-yl, C₁-C₄-alkoxycarbonyl, aminocarbonyl and C₁-C₃-alkylaminocarbonyl,
or one of the salts thereof, solvates thereof or solvates of the salts thereof.

2. The compound according to claim 1, characterized in that
R¹ represents a group of the formula 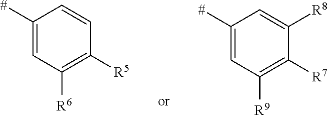
where # is the point of attachment to the nitrogen atom,
R⁵ represents 5-membered heteroaryl,
where heteroaryl may be substituted by a substituent selected from the group consisting of oxo and chlorine,
R⁶ represents hydrogen
R⁷ and R⁸ together with the carbon atoms to which they are attached form a 5-membered heterocycle,
where the heterocycle may be substituted by an oxo substituent,
R⁹ represents hydrogen
R² represents hydrogen
R³ represents hydrogen, fluorine, chlorine, methyl or trifluoromethyl,
R⁴ represents cyano, C₁-C₃-alkoxy, methoxycarbonyl, methylcarbonylamino, methylsulphonyl, —S(O)₂NR¹⁰R¹¹ or 5- to 7-membered heterocyclyl which is attached via a nitrogen atom,
where alkoxy may be substituted by a methoxy substituent, and
where methylcarbonylamino may be substituted by a —NH(CO)CH₂NH(CO)CH₂NH₂ substituent, and
where heterocyclyl may be substituted by 1 to 2 substituents selected independently from the group consisting of oxo and methyl, and
where
R¹⁰ represents hydrogen, C₁-C₃-alkyl, cyclopropyl or 4- to 8-membered heterocyclyl which is attached via a carbon atom,
in which alkyl may be substituted by 1 to 2 hydroxy substituents, and
in which heterocyclyl may be substituted by 1 to 2 methyl substituents,
R¹¹ represents hydrogen or C₁-C₃-alkyl, or
R¹⁰ and R¹¹ together with the nitrogen atom to which they are attached form a 4- to 7-membered heterocycle,
or one of the salts thereof, solvates thereof or solvates of the salts thereof.

3. The compound of claim 1, characterized in that
R¹ represents a group of the formula 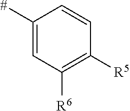
where # is the point of attachment to the nitrogen atom,
R⁵ is triazolyl or tetrazolyl,
where triazolyl may be substituted by a substituent selected from the group consisting of oxo and chlorine,
R⁶ represents hydrogen, or
R¹ represents 2,3-dihydro-1H-indazol-6-yl,
where 2,3-dihydro-1H-indazol-6-yl may be substituted by an oxo substituent,
R² represents hydrogen
R³ represents hydrogen, fluorine, chlorine, methyl or trifluoromethyl,
R⁴ represents —S(O)₂NR¹⁰R¹¹,
where
R¹⁰ represents hydrogen, C₁-C₃-alkyl, cyclopropyl or heterocyclyl which is attached via a carbon atom and selected from the group consisting of pyrrolidinyl and piperidinyl,
in which alkyl may be substituted by 1 to 2 hydroxy substituents, and
in which pyrrolidinyl and piperidinyl may be substituted by 1 to 2 methyl substituents,
R¹¹ represents hydrogen or C₁-C₃-alkyl, or
R¹⁰ and R¹¹ together with the nitrogen atom to which they are attached form a pyrrolidinyl,
or one of the salts thereof, solvates thereof or solvates of the salts thereof.

4. A method of making the compound of claim 1 of the formula (I) or one of the salts thereof, solvates thereof or solvates of the salts thereof according to claim 1, characterized in that a compound of the formula 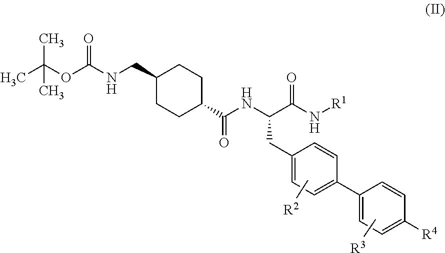
in which R¹, R², R³ and R⁴ have the meaning given in claim 1 is reacted with an acid.

5. A method for the treatment and/or prophylaxis of diseases using the compound of claim 1.

6. A method of making a medicament for treatment and/or prophylaxis of diseases using the compound of claim 1.

7. A method of making a medicament for the treatment and/or prophylaxis of thrombotic or thromboembolic disorders using the compound of claim 1.

8. A medicament comprising the compound of claim 1 in combination with an inert, nontoxic, pharmaceutically suitable excipient.

9. A method for treatment and/or prophylaxis of thrombotic or thromboembolic disorders using the medicament of claim 8.

10. A method for the treatment of thrombotic or thromboembolic disorders in humans and animals by administration of a therapeutically effective amount of the compound of claim 1.

11. A method for the treatment of thrombotic or thromboembolic disorders in humans and animals by administration of a therapeutically effective amount of the medicament of claim 8.

12. A method for the treatment of thrombotic or thromboembolic disorders in humans and animals by administration of a therapeutically effective amount of the medicament of claim 6.
